(19) United States
(12) Patent Application Publication
(10) Pub. No.: US 2019/0000349 A1
(43) Pub. Date: Jan. 3, 2019

(54) DIAGNOSIS TAILORING OF HEALTH AND DISEASE

(71) Applicant: Incyphae Inc., Scottsdale, AZ (US)

(72) Inventors: Sanjiv M. Narayan, Palo Alto, CA (US); Ruchir Sehra, Scottsdale, AZ (US)

(21) Appl. No.: 15/570,434

(22) PCT Filed: Jun. 28, 2017
(86) PCT No.: PCT/US17/39741

§ 371 Date: Oct. 30, 2017

Related U.S. Application Data
(63) Continuation of application No. 15/636,056, filed on Jun. 28, 2017.

(51) Int. Cl. A61B 5/08; A61B 5/00; A61B 5/145; A61B 5/113; G16H 50/20; G16H 50/50; A61B 5/053; A61B 7/00; A61B 5/01; G16H 10/20; G16H 20/30; A61B 5/024

(52) U.S. Cl. CPC A61B 5/08; A61B 5/024; A61B 5/0823; A61B 5/0826; A61B 5/4818; A61B 5/14542; A61B 5/113; G16H 50/20; G16H 50/50; A61B 5/0533; A61B 7/003; A61B 5/01; A61B 5/4806; G16H 10/20; G16H 20/30; A61B 5/7275

(57) ABSTRACT

The present invention relates generally and specifically to computerized devices capable of diagnosis tailoring for an individual, and capable of controlling effectors to deliver therapy or enhance performance also tailored to an individual. The invention integrates sensors which sense signals from measurable body systems together with external machines, to form adaptive digital networks over time of general health and health of specific body functions. The invention has applications in sleep and wakefulness, sleep-disordered breathing, other breathing disturbances, memory and cognition, monitoring and response to obesity or heart failure, monitoring and response to other conditions, and general enhancement of performance.

This application incorporates by reference the entire subject matter and application of attorney docket #2480-2 PCT (application PCT/US15/46819, filed Aug. 25, 2015) as well as attorney docket #2480-3 PCT (application PCT/US15/47820, filed Aug. 31, 2015).

### FIELD

The present invention relates generally and specifically to computerized devices capable of diagnosis tailoring for an individual, and capable of controlling effectors to deliver therapy or enhance performance also tailored to an individual. The invention integrates sensors which sense signals from measurable body systems together with external machines, to form adaptive digital networks over time of general health and health of specific body functions. Measurable body systems include the central and peripheral nervous system, cardiovascular system, respiratory system, skeletal muscles and skin as well as any other body systems that are capable of producing measurable signals. External machines include diagnostic sensors, medical stimulating or prosthetic devices and/or non-medical devices which may be consumer devices. The invention has applications in sleep and wakefulness, sleep-disordered breathing, other breathing disturbances, memory and cognition, monitoring and response to obesity or heart failure, monitoring and response to other conditions, and general enhancement of performance. This disclosure outlines several applications of this invention, using as an example methods and systems to enhance sleep-related bodily functions for use in normal individuals or patients with sleep-breathing disorders.

### BRIEF DISCUSSION OF RELATED ART

The human body has long been interfaced with artificial devices or machines. Prosthetic limbs have for centuries been made of wood combined with metal and other materials. Through recent technological advances, devices often now have sophisticated materials, design and control for a specific purpose—such as a robotic limb (see for instance singularityhub.com/2013/07/24/darpas-brain-controlled-prosthetic-arm-and-a-bionic-hand-that-can-touch) or a glucose-sensing insulin infusion pump.

Many body tasks are mediated by the brain (central nervous system) and/or peripheral nervous system. These functions include classical “neurological” functions such as vision or hearing, but also nearly all activities of daily life, including learning, moving, or operating machinery. Some tasks are mediated by systems other than the central and/or peripheral nervous system, and many tasks are performed by a combination of nervous system and non-nervous system tasks.

In many situations, the body's ability to perform tasks is constrained. Constraint can take many forms and may be functional or biophysical. Functional constraints may include a classical disease, such as stroke that directly restricts an individual's ability to move the foot. Functional constraints may also include underperformance on a task due to insufficient training, knowledge or acquisition of skills, or through disuse. Other functional constraints include normal or abnormal function of other body systems, such as fatigue from sleep-disordered breathing which restricts muscular function. Biophysical constraints include an external obstacle preventing movement of a limb in an enclosed space such as may affect a warrior or scuba diver, cold or heat or other forms of electromagnetic effect which prevent muscle motion. A biophysical constraint may also overlap with disease, such as loss of a limb from amputation which falls into both categories.

What is currently lacking is how devices can be used to “intelligently” tailor monitoring of health, or delivery of therapy to restore lost function, or enhance an existing function in a specific individual. This inability for prior and current devices to automatically monitor health, tailor therapy, and/or restore or enhance a function is striking when examining how easily the normal human brain senses, integrates and controls bodily functions.

The prior art has extensively studied, yet imprecisely defined, which regions of the brain or nervous system control bodily tasks, and how they interact with other physiological functions (e.g. organ systems) in a network. Simple bodily tasks, such as moving the biceps of the left arm or sensing from the right index finger, are well defined and often conserved between individuals. Nevertheless, functional mapping or “atlases” are debated even for “simple sensations” such as visual recognition of a face. Other bodily functions including “higher cortical” functions are neither well defined nor conserved. These complex bodily functions include healthy breathing, sleep, cognition, memory, mood, alertness, sensory-motor activities, and many other functions.

Currently, machines that attempt to modulate bodily functions are often based on a detailed knowledge of physiology, which for the brain may include neuroimaging, mapping of the brain and peripheral nerves for both normal and abnormal function. Unfortunately, such detailed knowledge is typically incomplete. In part, this is because mapping of locations of the brain for normal and abnormal tasks often vary between individuals, and may vary for the same person at different times. Regions of the brain and other systems that mediate many body functions are thus poorly understood. This includes sleep control, breathing control, memory, cognition, mental performance and others. Even for apparently well-understood (or well “mapped”) functions, physiological studies raise additional uncertainties such as variations over time based on the functioning of other systems or the health condition of a particular individual.

We define a functional domain as the aggregation of all the elements relating to a distinct bodily function, sometimes associated with a specific organ system or a combination of systems that results in the overall function, e.g., breathing. Mapping functional domains of a bodily function is difficult, particularly for functions involving the brain. However, there is an urgent need to sense and modulate functional domains whose altered function may cause disease or suboptimal performance.

In traditional theory, sleep and wakefulness are modulated by brain regions including the posterior hypothalamus, while memory is encoded by the hippocampus and other regions of the limbic system. However, it is not clear what brain regions are responsible for controlling sleep, or for mediating abnormal breathing in sleep apnea. Regions of the brainstem that control single airway muscles are better characterized, such as nuclei for the hypoglossal nerve (twelfth cranial nerve) that controls tongue movement. Yet, how such nuclei are involved in complex functions, such as abnormal breathing to produce obstructive sleep apnea, is not understood. As a result, it has been difficult to treat this condition or discover novel systems to physically or electrically modulate single muscles such as the tongue to reduce obstruction.

Sleep is a bodily function that integrates the nervous system, skeletal muscle, cardiopulmonary, and other body systems. Sleep alternates with and enables subsequent wakefulness, and is required for normal functioning of most organ systems. Sleep is traditionally considered to be controlled by specific regions of the brainstem (primitive brain) that regulate and are modulated by function of the higher brain (cerebral cortex). These regions, in turn, control muscles of breathing, other involuntary muscles such as sphincters of the gastrointestinal and genitourinary tracts, voluntary muscles such as muscles of the legs or arms, sensory function, and other bodily functions.

Much work over several decades has strived to define which regions of the brain mediate the complex bodily function of sleep. As outlined above, while functional mapping is well defined for “simple” functions such as controlling a defined muscle (e.g., the biceps of the upper arm) or sensation (e.g., the right index finger), it is far less clear for sleep. Interactions between the multiple organ systems impacted by sleep further complicate precise mapping.

An individual's ability to sleep may be compromised in many ways. Among the most important and common are sleep hypopnea (reduced breathing) and apnea (absence of breathing), in which impaired breathing in sleep interrupts sleep functioning, as well as primary sleep disorders such as insomnia, where the individual cannot sleep efficiently or sufficiently. Sleep disorders often negatively impact wakefulness, resulting in daytime drowsiness that impairs daily activities. Sleep disorders can also lead to disorders from breathing such as low oxygen and/or high CO₂ levels with metabolic effects including acidosis, disorders of the heart such as failure and abnormal rhythms, disorders of the immune system causing susceptibility to infection, psychological disorders such as stress, depression and other mood disorders, anxiousness and psychosis, as well as several other states of poor functioning and disease.

Sleep apnea may be obstructive or central. Obstructive sleep apnea (OSA) is increasingly recognized in individuals who snore, who are overweight and who may develop sequelae such as heart failure. However, OSA remains under-diagnosed, and may occur in individuals without these classical features. Central sleep apnea is also common, under-recognized and associated with comorbidities such as heart failure. It is likely that central sleep apnea (CSA) also occurs alongside obstructive sleep apnea, since treatments that physically open the throat muscles and prevent obstruction may sometimes leave residual apnea. Many patients with OSA develop some component of CSA over time if left untreated.

Obstructive sleep apnea results from partial or complete airway collapse in sleep. Central sleep apnea results from reduced brain stimulation of the respiratory muscles in sleep. Both forms are typically diagnosed using overnight polysomnography (PSG), a test that typically measures at least eight (8) sensor channels including the electroencephalogram (EEG), electro-oculogram (EOG), electrocardiogram (ECG), chin electromyogram (EMG), nasal and oral airflow, respiratory “effort,” oxygen saturation (SaO₂ or sat), and body position. Unfortunately, the PSG is often considered a cumbersome test, performed in the unnatural conditions of an overnight laboratory stay attended by expert technicians and, sometimes, physicians. The traditional PSG is not well liked or tolerated by patients, cannot easily be repeated to assess the impact of therapy and cannot be performed at home.

From a polysomnogram, apnea is defined as absence of breathing (decrease of nasal/oral airflow, a surrogate measure of tidal volume, by at least 90%) for at least 10 seconds, while hypopnea is defined as decrease in airflow of at least 30% for at least 10 seconds accompanied by at least a 3% decrease in oxygen saturation and/or terminated by arousal from sleep. Apnea is defined as obstructive if accompanied by additional inspiratory effort against the occluded airway, as measured via EMG and chest sensor. Without such accompanying effort, apnea is defined as central. Similarly, hypopnea is obstructive if there are signs of upper airway flow limitation, and is otherwise considered central. The apnea-hypopnea index (AHI) is the total combined number of apnea and hypopnea episodes per hour of sleep, and is typically classified as no sleep apnea (AHI <5), mild sleep apnea (AHI of 5-14), moderate sleep apnea (AHI of 15-29) and severe sleep apnea (AHI ≥30).

Several treatments are available for obstructive sleep apnea, but these are often not well tolerated. The most commonly used treatment currently is continuous positive airway pressure (CPAP) to keep the airway open and reduce/eliminate obstruction. Other options include mechanical splints such as oral appliances, and even surgical procedures to reduce/eliminate obstruction. Some recent devices have applied stimulation to the muscles of the tongue or face to eliminate obstruction, but it is unclear how well they will work in the broad population.

A few strategies have been proposed to improve central sleep apnea—or more generally the central control of sleep. CPAP and assisted servo ventilation are commonly used but very poorly tolerated. Certain stimulant medications are sometimes helpful but often contraindicated in patients with other comorbidities. Recently, one investigational device (Remede® by Respicardia) has being studied to pace the phrenic nerve in order to stimulate the diaphragm to breathe [Costanzo MR et al. Lancet 2016]. Since central sleep apnea may relate directly to sleep disorders, treatments for central sleep apnea may potentially also help other conditions. It is increasingly appreciated that central sleep apnea makes heart failure worse, and so treatment for central sleep apnea may improve symptoms of heart failure, and other cardiac and non-cardiac conditions such as insomnia and psychological sequelae.

Pharmacological drug therapy is often used to induce sleep, but these agents are not useful in sleep apnea. Most such drugs rarely mimic the natural stages of sleep, rarely induce rapid eye movement (REM) sleep that is essential for restfulness, and may paradoxically worsen sleep disorders and produce daytime drowsiness despite nighttime unconsciousness.

New therapeutic modalities are clearly needed to modulate the complex functions outlined above—often including a component of central or peripheral nervous system involvement. Emerging modalities involve electrical stimulation/modulation of brain or nervous system activity, typically at a specific target region. All these current modalities suffer from a significant common problem, as they attempt to perform therapy with no or minimal sensory input, feedback, or modulation of such therapy based upon the individual patient's neurological activity.

One example of electrical stimulation therapy is noninvasive or minimally invasive trigeminal nerve stimulation (e.g., NEUROSIGMA®) that is being evaluated to treat depression and seizures. Unfortunately, the true mechanism of action of such therapy is unclear. Whether this is due to the actual trigeminal nerve being stimulated, direct stimulation of the frontal lobe of the brain, indirect inhibition of cerebral blood flow or some other as yet unknown mechanism, still remains to be determined and will affect the ability of such therapy to be applied successfully. Additionally, this therapy is applied as a “one-size-fits-all” approach without any adaptation for individual patient responses.

Other non-invasive neuromodulation/stimulation approaches are also being considered include stimulation of the vagus nerve for seizures (Carbomed, Inc.). Similar to trigeminal nerve stimulation, the mechanism is poorly understood, the actual stimulation of the vagus nerve is unclear via this noninvasive approach, and there is no individual patient adaptation. A number of technologies are attempting to treat depression via noninvasive transcranial application of an electrical and/or magnetic field (Neuronetics Inc., Neosync Inc., Brainsway Inc., Cervel Neurotech Inc., and Tal Medical Inc.).

For apnea, approaches that try to modulate obstructive sleep apnea, including stimulation of the hypoglossal nerve (Inspire Med Inc.) or other throat muscle (Apnex Medical Inc.)—are being evaluated but typically do not have individual patient-tailored therapies. In fact, whether direct management of the obstruction resolves the problem of apnea is also unclear due to commonality of a central sleep apnea component in most patients.

Other invasive approaches to neuromodulation include vagal nerve stimulation to treat seizures and depression (Cyberonics), spinal cord stimulation to treat pain (such as Medtronic Inc., Boston Scientific Inc., Advanced Neuromodulation Systems Inc.), direct deep brain stimulation to treat seizures (Medtronic Inc., Boston Scientific Inc., others) or even cognitive disorders (Thync Inc.). However, these therapies target single components of the physiologic network for a bodily function, and are limited because they do not consider other physiological systems (other portions of the “physiological network”) that cause abnormal functioning. This may lead to suboptimal therapy, compensatory mechanisms that further diminish the efficacy of therapy, or unwanted effects. Moreover, these therapies are only as good as the accuracy of their specific targets, and brain/nerve regions are imprecisely defined for many bodily functions including sleep control, sleep-breathing conditions, cognition, alertness, memory, overall mental performance, or response to obesity.

Thus, for apnea, while these current approaches show interesting preliminary data, they all suffer from the same problems—namely, poor understanding of mechanism, poor patient-tailoring of therapy, and suboptimal therapy feedback and adaptation for individual patients needs.

Traditional therapies have also not typically been effective for managing central sleep apnea, other cognitive or performance functions, alertness, heart failure or obesity.

Devices can be used for other functions, such as the increasing use of virtual environments. Here, the goal is usually to create an illusionary or representative environment by feeding specific sensory inputs (primarily visual, tactile and/or auditory) to simulate or replicate real-world experiences. However, such approaches are severely limited in that pathway for normal functioning, as well as those for abnormal function, can vary dramatically from individual to individual. Thus, the sensory inputs or outputs in the virtual environment often will not accurately represent nor simulate that function for an individual.

Devices can be used in many other applications to enhance or compensate for other functions such as motor tasks which are limited or constrained. Devices can address physical constraints such as an external obstacle, or compensate for physical loss e.g. of a limb from amputation. As discussed, devices can be used for central or obstructive sleep apnea, but with limited success.

Many attempts have been made to develop devices to address functional constraints or limitations, based on the paradigm that body sensors (e.g., the eye), nervous function (e.g., the central and peripheral nervous system) and effector organs (e.g., a muscle group) can be functionally mapped to specific anatomic locations. These solutions are limited largely because the precise locations of the brain (“atlases”) or other physiological systems that mediate each task are not well defined, particularly for complex functions. Much data has come from animal models that are not well suited to model or analyze complex human functions or mental functions.

It would be of great benefit to society to develop a device which can enhance bodily tasks tailored to an individual, which can sense health or disease in that specific individual, can do so without invasive testing which may enable repeated testing, and can also be used to modulate that bodily task in that individual. An example would be a device to detect sleep disturbance in a specific individual to restore sleep functionality, i.e., to prevent or treat obstructive or central sleep disorders. A device to enhance wellness including alertness, breathing, sleep, motor activity or even some aspects of neural functioning tailored to an individual, in whom these functions are not diseased, would also be of great value. Currently, there are few methods in the prior art to achieve these goals

### SUMMARY OF THE PRESENT INVENTION

The present invention provides a method, device and system which overcome the deficiencies of the prior art and enhances the bodily tasks of an individual by sensing health or disease tailored to an individual, without invasive testing and which is able to modulate functional components for that bodily task tailored to that individual. More specifically, in a specific embodiment the present invention provides a method, device and system which detect sleep disturbance in a specific individual and is tailored to that individual to restore sleep functionality in that individual, i.e., to prevent or treat obstructive or central sleep disorders. The present invention also provides a method, device and system that enhances tasks such as alertness, breathing, sleep, motor activity or even some aspects of neural functioning tailored to an individual, in whom these functions are not diseased.

The current invention creates a dynamic digital representation of health or disease over time for an individual, known as an enciphered functional network. This network is tailored to an individual by using sensed information from multiple physiological systems that mediate a given bodily function, and can be used to modulate functionality of that task, tailored to an individual. The invention departs from the prior art in many ways. First, the invention has the capability to monitor important bodily tasks for an individual repeatedly and non-invasively. This enables implementations wholly or in part using consumer devices such as smartphones, home motion sensors, consumer cameras or microphones. The invention is thus connected to the internet of things (IofT). Second, the invention is focused on the enciphered functional network (EFN), an individualized, digital representation of normal and/or diseased bodily functions, which is used to detect perturbations or produce enhancements tailored to that individual to modify functions accordingly. The EFN does not by definition require detailed a priori physiological or mechanistic definitions of the bodily task, which are often unavailable for complex tasks such as sleep, alertness, weight maintenance, maintenance of body fluid equilibrium in patients with heart failure, or neurological tasks. Instead, the EFN is constructed by repeated sensed measures referenced to different states of that body function in an individual, and thus represents that body function as sensed signatures—which may be normal or abnormal. Third, the invention is able to enhance performance or re-instate lost functions tailored to the individual using the enciphered functional network. Fourth, the invention uses a combined biological and machine approach.

For the purposes of this disclosure, the following definitions apply.

Associative learning is defined as the process of linking sensed signatures and other inputs, with a body task. Sensed signatures are typically from body systems. For this disclosure, body tasks are typically complex tasks rather than reflexive or other simple tasks. Associative learning may be iterative, such that associations are modified (“learned”) based upon patterns of change between these processes. An example includes associating high electrical impedance across the chest wall (i.e. greater content of the insulator, air) with abnormal breathing.

Bodily function is defined as the processes needed to perform a task, that may include physiologic or pathological processes. Bodily function is typically complex with non-limiting examples such as sleep, sleep apnea, mental performance, or the response to obesity. Bodily functions involve a network of functional domains that may interact, each of which may include the brain and central nervous system, peripheral nervous system, cardiovascular, pulmonary, gastrointestinal, genitourinary, immune, skin and other systems. A bodily function may result from biological activity/function, and may be modulated by a non-biological or artificial component, e.g., reading with glasses, driving, using remote control unit, a patient moving a combined natural/cybernetic limb, etc. A bodily function can be represented by several bodily signals. For instance, the bodily function of breathing may be represented by sensed signals of breathing rate, breathing depth, variations in heart rate, oxygenation level on the skin and the chemical balance of sweat, among others.

Bodily signal means signals generated by and/or sensed from a human, animal, plant, bacterial or other single-cell-based body or multi-cell-based body. For purposes of this definition, viruses and prions are included. Bodily signals particularly include signals generated by and/or sensed from the human body. Bodily signals are generally associated with bodily functions. The term “non-bodily signal” indicates that it is generated from a source other than a single- or multi-cell-based body. Examples include an external “signal” from an external electrical source, machine, sensor, etc. When the term “signal” is used without the term “bodily” or “non-bodily”, the term “signal” indicates that it includes both “bodily signals” and “non-bodily” signals, i.e., it includes all signals.

Body means the physical structure of a single-celled organism, a multi-celled organism, viruses and prions. Organisms include animals (such as, but not limited to, humans), plants, bacteria, etc.

Consumer device is defined as a device that is available directly to a consumer without a medical prescription, and is typically not regulated by a medical regulatory agency or body, such as the U.S. Food and Drug Administration or similar Regulatory Bodies in other Nations, which may include hardware, software or a combination. A consumer device is not a medical device, the latter which is defined as an instrument, apparatus, implement, machine, contrivance, implant, in vitro reagent, or other similar or related article, including a component part, or accessory, which is intended for use in the diagnosis of disease or other conditions, or in the cure, mitigation, treatment, or prevention of disease, in man or other animals. The definition of a medical device excludes medical decision support software.

Customizing of analysis or therapy are performed using a computerized framework entitled the “enciphered functional network”, to maintain health functions or prevent disease. Customizing is dynamic and occurs at many levels including deciding which sensors to apply in an individual, where to apply it/them, which to combine for a specific task, how to analyze them dynamically, i.e. over time, and how to deliver an effector response if undesired signal patterns are detected.

Disturbance of the sensed signal, in the preferred embodiment of breathing related signals, is associated with partial or full arousal from sleep, or partial or full arousal from an apnea event.

Effector is defined as a means of performing a bodily task, and may include a physical appliance, prosthesis, mechanical or electronic device. A physical appliance may enhance a bodily function, such as a device to move a limb or move the diaphragm to enhance breathing during sleep or a splint to keep the airway open during sleep, or one or more signals to stimulate a bodily function, such as electrical stimulation of the phrenic nerve to enhance breathing during sleep, or an artificial prosthesis such as a cybernetic limb or implanted circuit for the peripheral or central nervous system.

Effector response is the result of the effector to partially or fully complete or enhance a bodily task. For instance, if the bodily task is improvement of sleep disordered breathing, the effector of altering lighting may have the effect of shifting sleep phase; the effector of introducing an auditory signal (e.g. specific musical rhythm, metronome) may have the response of improving breathing. As another example, if the effector is stimulation of the triceps muscle in the arm, the effector response may be to extend the arm by 30 degrees, while the entire task may be to fully straighten the arm.

Effector signal is the signal delivered by the effector to produce the effector response.

Enciphered network or enciphered functional network (EFN) is defined as a model associating measured parameters (sensed signatures) with aspects of the bodily task including effectors and other sensors. In the preferred embodiment, the EFN is a computerized representation of one or more bodily tasks in an individual. The EFN encompasses patterns of fluctuation in health and disease for that bodily task for that individual, ideally under varying circumstances over time to capture multiple ‘state spaces’ of that function in that individual. The EFN for the same task may thus differ between individuals. The EFN represents components of the bodily task, i.e. functional domains, that can be constructed even if physiological knowledge of the tasks is incomplete—which is often the case. The EFN can be represented in symbolic code, in which case it may be a mathematical or other abstract representation. The EFN may include sensors, computational elements, storage elements, effectors and associated hardware and software. If applied to the nervous system, the EFN is termed an enciphered nervous system. The EFN contrasts with the prior art in which published data across many individuals define laboratory cutpoint values that are then used to estimate health and disease in each individual with varying success.

Encipher is defined as the process of coding information.

Enhanced performance or enhancement is defined as improvement to the normal healthy and non-diseased baseline function in an individual. Enhanced performance thus would not include therapies for disease such as pacing in an individual with abnormally slow heart rates or in a patient or an insulin pump in known diabetic patients.

External machine is defined as a mechanical, electrical, computational or other non-natural (native biological) device. This may be external to the body but can be in contact with or implanted within the body.

Extremity of the body is defined as limbs and associated structures of the body including arms, legs, hands, feet, fingers, toes, and subsegments thereof.

Functional domain is defined as the elements relating to a bodily task. This may include sensed elements, analysis elements and effector elements. Analysis elements may be “learned”, preprogrammed, reflexive, or passive. Each element may be biological, non-biological or artificial. A Functional domain is thus the aggregation of all elements relating to a bodily task, which may comprise ‘measurable body systems’ such as the nervous system, the heart and cardiovascular system, blood vessels, the lymphatic system, interstitial tissue planes, endocrine (hormonal) organs. The functional domain comprises multiple organs, which may provide senses signatures and/or serve as targets for effector therapy. This departs from traditional attempts to detect markers of precise mechanisms, which may work for simple tasks (e.g. limb movement in a reflex arc, elevation of troponins to signal a heart attack) but are far more difficult for complex tasks (e.g. breathing, alertness, weight control).

Functional domains are well defined for a simple task such as sensation at the shoulder. In this case, the functional domain is a sensed “dermatomal distribution” mediated by sensory nerves from the C435 distribution, and effectors at the shoulder including motor nerves and muscles. As another simple component of the task of breathing is the movement of the diaphragm which is controlled by the phrenic nerve (spinal distribution C3-5). It should be noted, however, that even simple domains may be more complex, e.g. shoulder sensation from these nerves may be mimicked (‘activated’) by heart pain (angina pectoris), since these nerves also supply the heart.

Several functional domains are typically involved in monitoring, tracking or effecting changes in a complex task. In the preferred embodiment, a complex bodily task is typically represented by several functional domains. The task of breathing, for instance, reflects functional domains including: cerebral inputs and circadian rhythms at the brainstem (potentially measurable via the EEG or nerve activity), phrenic nerve or intercostal nerve activity (potentially measurable directly by electrical activity, or indirectly by chest wall motion), oxygenation (potentially measurable from blood or skin oxygenation, skin color), or heart rate changes (termed ‘sinus arrhythmia’).

Separate functional domains may be defined which reflect natural biological activity including breathing, alertness, sleeping, dreaming, maintenance of weight, maintenance of body fluid content, beating of the heart, walking, running.

Functionally associated is defined as sensed signals or functional domains that occur when that function occurs. An example is activity in portions of the brain controlling breathing with activity in muscles of breathing such as the intercostal muscles or diaphragm. Functional association does not need to be part of a mechanistic cascade, even though it can be used to track that biological mechanism. For example, sensed activity in shoulder nerves is associated with heart pain (angina) and can be used to track angina in some individuals, yet shoulder nerve activity is not part of the biological mechanisms causing coronary disease.

Machine learning is defined as a series of analytic methods and algorithms that can learn from and make predictions on data by building a model rather than following strictly static programming instructions. Another definition is the ability of computers to learn without being explicitly programmed. Machine learning is often classified as a branch of artificial intelligence, and focuses on the development of computer programs that can change when exposed to new data. In the current invention, machine learning is one tool that can be used to create the enciphered functional network linking sensed signatures with bodily tasks in each individual, i.e. for a personalized solution to maintain health and diagnose disease. Machine learning can take many forms including artificial neural networks, and can be combined with heuristics, deterministic rules and detailed databases.

Medical device is defined as an instrument, apparatus, implement, machine, contrivance, implant, in vitro reagent, or other similar or related article, including a component part, or accessory, which is intended for use in the diagnosis of disease or other conditions, or in the cure, mitigation, treatment, or prevention of disease, in man or other animals. The definition of a medical device excludes medical decision support software.

Mental alertness is defined as an awake state that focuses on a specific task, that can be measured by performance at that task. Improved mental alertness is characterized by being awake and performing mental and other tasks well. Reduced mental alertness can include many states that include but are not limited to impaired performance of a task, “mental fatigue”, loss of focus, attention deficit, somnolescence, sleepiness, narcolepsy, sleep and disease processes that include the above as well as coma, “fugue” state and others.

Metabolic health includes glucose handling and derangements (including diabetes mellitus), weight management and its derangements (including obesity), fluid management and its derangements (including edema and decompensated heart failure), deconditioning (including acidosis and low pH in sweat on exertion) among others.

Physiological Function includes but is not limited to breathing rate, breathing effectiveness, heart beating rate, heart beating effectiveness, alertness, maintenance of optimal weight.

Sensed signatures are defined as one or more signals from sensors related to a bodily task. In aggregate, sensed signatures are used to define a functional domain and/or a bodily task, including the very important phenomenon of fluctuations over time that are specific to an individual. Sensors may be biological, non-biological or artificial. Sensed signatures may include physiological data as well as data from symptoms or physical examination. For instance, the task of breathing may be represented by a nerve domain, with sensed signatures of firing rates of sympathetic nerves or nerves supplying the pharyngeal muscles; a lung functional domain, with sensed signatures including skin oxygenation and movement of the chest wall a heart domain, with sensed signatures including heart rate, sinus arrhythmia and variations in pulse amplitude. Sensed signatures for a complex bodily task typically vary for each individual. For instance, in tracking sleep disordered breathing, sensed signatures of heart rate will be less important in some patients e.g. with atrial fibrillation, sensed signatures of chest wall movement may be less important in others, e.g. those with predominantly hypopnea/arousals as opposed to those predominantly with apneas, or those who perform abdominal breathing; sensed signatures of oxygenation may be difficult to assess in patients with peripheral vasoconstriction.

Signals can be defined as either sensed or acquired. Sensed signals are detected unaltered from their natural form (i.e. recorded) with no transformation. Sensed signals can be detected by humans (e.g. sound, visual, temperature) but also machines such as microphones, auditory recorders, cameras, thermometers). Acquired signals are detected in a transformed state, such as an ECG recording. The distinction between sensed and acquired signals is one way to classify embodiments of the invention that use consumer devices (sensed signals) versus medical devices (acquired signals).

Response signals are similar to effector signals, which control effectors in the invention to return an index of health towards desired levels for an individual. If an index of breathing health indicates apnea, one response signal will control a response device to stimulate breathing. If an index of metabolic health indicates weight gain, one response signal will be a message to eat less.

Smart data is defined as application-specific information acquired from multiple sources that can be used to detect normal and abnormal function in that application. Smart data is thus different from the term “big data”. Smart data is tailored to the individual, and tailored to address the specific task or application—such as to maintain health and alertness or detect and treat disease such as sleep disordered breathing, Tailoring is based on knowledge of what systems may impact the task in question. This knowledge may be based on physiology, engineering, or other principles. Conversely, “big data” is often focused on “big” datasets for the goal of identifying statistical patterns or trends without an individually tailored link.

Smart data in this invention uses readily available signal sources which are ideally acquired repeatedly and even near-continuously. Such signals and smart data acquisition are by definition mostly non-invasive. This approach is well suited to use signals from consumer level devices on movement, vibration, sound, electrical signals, optical reflectance, heat among others. Smart data analysis will use the enciphered functional network in several modes, including heuristics, machine learning, artificial intelligence, fuzzy logic, database lookup. Another way to look at smart data is to use detailed mechanistic or observational data in an individual, and apply this broadly to populations of individuals, using the enciphered functional network to tailor the specific analysis or intervention to each individual. This process can be termed “digital decision” making, or “digital judgment” and has no analogue in the prior art. It extends the subjective clinical decision making by making it objective, reproducible and based on sensed signatures from state-of-the-art sensors.

Symbolic model herein is a mathematical representation linking measured sensed activity with a functional task even if complete physiological descriptions for that task are lacking. It is the underlying representation of the enciphered functional network. It can also be termed a symbolic representation. This may include analog recorded physiological signals, digital coded ciphers, computer code, visual representations such as photographs or graphics, auditory coding such as patterns of clicks or sounds or music, and so on, and can be used to aid in rapid, clear transformation of data to monitor or modify a specified task.

“Task” means a piece of work, action or movement to be done, completed or undertaken. The term “bodily task” means a piece of work, action or movement to be done, completed or undertaken by a “body”, defined herein.

Therapeutically effective is defined as an effector function or dose of an intervention or therapy that produces measurable improvement in one or more patient outcomes. An example would be patterns of energy directed to the scalp to stimulate target regions controlling breathing, in order to treat central sleep apnea. Ideally, an intervention will minimize impact to other regions of the body, in this case the scalp which may be achieved by a small contact device rather than a cap that encompasses the entire scalp, or focusing energy from a non-contact device on the target region and not the entire head.

Other biological terms take their standard definitions, such as heart failure, tidal volume, sleep apnea, obesity and so on.

This invention creates an enciphered functional network. The potential uses of this invention are broad and include the following. Detecting one or more signals, directly or indirectly, from one or more sensors, the signals associated with breathing at a plurality of points in time; tailoring a diagnosis of breathing-health to the individual based upon identifying one or more breaths from the one or more signals, and identifying at least one or more of (i) one or more quantitative indexes of health symptoms and (ii) one or more quantitative indexes of physical examination signs; wherein the diagnosis tailoring is determined using one or more of mathematical weighting, machine learning, statistical correlation, and applying a threshold of breathing-health; and, providing a representation of the tailored diagnosis at the one or more points in time.

The invention presents a series of important innovations. It creates a computerized representation of a bodily function from various sensed signals, tailored to the individual, and uses this representation to maintain health and treat disease, i.e. it tailors the entire process of signal acquisition, signal analytics and diagnosis to effect therapy.

In a preferred embodiment of the invention, the enciphered functional network for a bodily task is further tailored to the individual by taking into account task-relevant symptoms or physical examination findings. This enables a truly personalized representation of health or disease for measured bodily task. Such representation can, for example, be displayed using one or more of a consumer device, a medical device, a computer, a medical record and a printed representation or other physical representation.

In one preferred embodiment, the enciphered functional network is optimized for breathing disorders. To monitor breathing health and disease, sensed signatures from multiple functional domains are complemented by data from indexes/scores of physical symptoms and examination findings. Symptom and examination scores may include the STOP-BANG, Berlin questionnaire for sleep apnea, Epworth sleepiness scale (ESS), Functional Outcomes of Sleep Questionnaire (FOSQ), or other scoring methods. These examples include assessment of sleepiness, activities of daily living and physical examination, and are provided by way of example, and other approaches may be applicable in the invention for those skilled in the art.

In another preferred embodiment, the enciphered functional network is optimized for heart function. To monitor cardiac health and disease, sensed signatures from multiple functional domains are complemented by data from indexes/scores of physical symptoms and examination findings. Symptom and examination scores may include the Canadian cardiovascular score for angina, the New York Heart Association scale for heart failure, or the American Heart Association heart failure grading system. These examples assess volume overload, functional status and physical findings. Other personalized information such as information from quality of life indexes can be incorporated, and are provided by way of example; other approaches may be applicable in the invention for those skilled in the art.

In another aspect, there is provided a method to enhance performance of a bodily task, the method including detecting signals associated with the task at one or more sensors, processing the signals to create one or more sensed signatures, processing the signatures using an enciphered functional network to determine one or more effector responses needed to enhance performance of the bodily task, delivering via the enciphered functional network one or more effector signals (the effector signals based on the one or more effector responses), and enhancing performance of the task.

In another aspect, there is provided a method for treating a disease, the method including detecting signals associated with one or more bodily functions at one or more sensors associated with the human body, processing the signals to create one or more sensed signatures of the one of more bodily functions, processing the signatures using an enciphered functional network to determine one or more effector responses needed to treat a disease, delivering via the enciphered functional network one or more effector signals (the effector signals based on the one or more effector responses), and treating the disease.

In another aspect, there is provided a method for transforming nerve activity associated with one or more bodily functions, the method including detecting bodily signals of nerve activity associated with the one or more bodily functions at one or more sensors, processing the bodily signals to create one or more sensed signatures of the one or more bodily functions, processing the signatures using an enciphered functional network to determine one or more effector responses needed to transform nerve activity, delivering via the enciphered functional network one or more effector signals (the effector signals based on the one or more effector responses), and transforming nerve activity.

In another aspect, there is provided a method for controlling a device using an enciphered functional network, the method including detecting bodily signals from a body using one or more sensors, processing the bodily signals to create a sensed signature, processing the sensed signature using an enciphered functional network to determine one or more effector responses to control the device, delivering via the enciphered functional network one or more effector signals (the effector signals based on the one or more effector responses), and controlling the device.

In another aspect, there is provided a method to measure bodily function in an animal, the method including detecting bodily signals associated with sensory activation, processing the bodily signals to create one or more sensed signatures, and processing the sensed signatures using an enciphered functional network to determine one or more effector responses needed to enhance the bodily function of the animal.

In another aspect, there is provided a method and system for improving performance of a specific human task, the method including selecting one or more functional domains for that task, identifying organ systems or regions of a human body associated with said functional domains, utilizing effector devices which can modify said functional domains, and measuring sensed signatures of said functional domains to monitor improvement of the specific human task.

In another aspect, there is provided a method and system for improving performance of a specific human task, the method including identifying one or more regions of a human body associated with parts of the brain that serve a specific function, placing low energy stimulating electrodes proximate to the one or more regions of the human body, applying stimulation through the electrodes to activate the parts of the brain, and measuring changes related to the parts of the brain to verify improvement of the specific human performance.

In another aspect, there is provided a method and system for enhancing attention, the method including selecting one or more functional domains associated with attention, monitoring sensed signatures from said functional domains and applying stimulation with one or more effector devices to modulate said functional domains to enhance attention. Functional domains associated with attention include a brain domain with sensed signatures including the scalp EEG, scalp temperature; a central and peripheral nervous domain with sensed signatures including sympathetic nerve system activity, peripheral nerve activity; a skin domain with sensed signatures including pilierection (hairs standing up); a heart domain with sensed signatures including heart rate, pulse volume, cardiac contractility measures; a lung domain with sensed signatures including breathing rate, breathing depth, oxygenation; an eye domain with sensed signatures including pupillary diameter, pupillary fluctuations, scleral color; an endocrine domain with sensed signatures of thyroid or adrenocortical systems; a musculoskeletal domain with sensed signatures including muscle tone, muscle oscillations, muscle response to stimuli (reactivity), and others.

In another aspect, there is provided a method and system to modulate or enhance attention, the method involving delivering effector responses using consumer de vices or other devices to modulate functional domains associated with alertness. In one embodiment, effector responses may be applied in the skin domain such as delivering cold, hot and vibration stimuli to alter alertness.

In another embodiment, the method to enhance alertness includes selecting one or more regions of the central or peripheral nervous system domains associated with attention, and applying low energy stimulation through electrodes to activate parts of a patient's central nervous system and/or peripheral nervous system to enhance attention and/or treat an attention disorder.

In another aspect, there is provided a method and system for improving performance of sleep, the method including selecting one or more functional domains for sleep, identifying effector systems associated with said functional domains, utilizing effector devices to deliver stimuli to modify said functional domains, and measuring effector responses to monitor improvement of sleep. Functional domains for sleep include but are not limited to brain, central and peripheral nervous system, lung, heart, endocrine. Sensed signatures of the brain domain for sleep include, but are not limited to, scalp electrical signals and EEG, scalp temperature. Sensed signatures of the peripheral nerve domain for sleep include, but are not limited to, rates and patterns of peripheral nerve firing, rates and patterns of pharyngeal muscle nerve firing, rates and patterns of phrenic nerve activity. Sensed signatures of the lung domain for sleep include, but are not limited to, sound produced by sleep-breathing (e.g. normal breaths snoring), chest movement rate and depth. Sensed signatures of the peripheral muscular domain for sleep include, but are not limited to, body movement on external motion sensors. Sensed signatures of the skin domain for sleep include, but are not limited to, skin oxygenation patterns, regional temperature in the face/torso/periphery; regional skin impedance in the face/torso/periphery; regional chemical composition (sodium, others) in the face/torso/periphery. Sensed signatures of the heart domain for sleep include, but are not limited to, heart rates and variability in heart rate during sleep. Components of the polysomnogram during sleep can also be sensed signatures and include brain (EEG), eye movements (EOG), muscle activity or skeletal muscle activation (EMG), heart rhythm (ECG) respiratory airflow and respiratory effort and peripheral pulse oximetry.

In another aspect, there is provided a method and system for treating a sleep disorder, the method including selecting one or more functional domains for sleep, identifying effector systems associated with said functional domains, utilizing effector devices to deliver stimuli to modify said functional domains, and measuring effector responses to monitor improvement of sleep. Functional domains for sleep disorder include but are not limited to brain, central and peripheral nervous system, lung, heart, endocrine. Sensed signatures of the brain domain for sleep disorder include, but are not limited to, scalp electrical signals and EEG, scalp temperature. Sensed signatures of the peripheral nerve domain for sleep disorder include, but are not limited to, rates and patterns of peripheral nerve firing, rates and patterns of pharyngeal muscle nerve firing, rates and patterns of phrenic nerve activity. Sensed signatures of the lung domain for sleep disorder include, but are not limited to, sound produced by sleep-breathing (e.g. normal breaths snoring), chest movement rate and depth. Sensed signatures of the peripheral muscular domain for sleep disorder include, but are not limited to, body movement on external motion sensors. Sensed signatures of the skin domain for sleep disorder include, but are not limited to, skin oxygenation patterns, regional temperature in the face/torso/periphery; regional skin impedance in the face/torso/periphery; regional chemical composition (sodium, others) in the face/torso/periphery. Sensed signatures of the heart domain for sleep disorder include, but are not limited to, heart rates and variability in heart rate during sleep. Components of the polysomnogram during sleep can also be sensed signatures and include brain (EEG), eye movements (EOG), muscle activity or skeletal muscle activation (EMG), heart rhythm (ECG) respiratory airflow and respiratory effort and peripheral pulse oximetry.

In another aspect, there is provided a method and system for treating a sleep disorder, which modulate sleep cycles including, but not limited to, delivery of light, delivery of electrical, auditory or heating stimuli, modulation of breathing by stimulation of nerves or muscles of breathing, modulation of neck and pharyngeal muscles by electrical stimulation to reduce snoring. The method may also include selecting one or more regions of a patient's central nervous system and/or peripheral nervous system associated with sleep disorder, and applying low energy stimulation through electrodes to activate the patient's one or more regions of central nervous system and/or peripheral nervous system to treat the sleep disorder. Other interventions will be apparent to those skilled in the art.

In another aspect, there is provided a method and system for improving performance of breathing, the method including selecting one or more functional domains for that task, identifying effector organ systems or regions of a human body associated with said functional domains, utilizing effector devices which can modify said functional domains, applying stimulation through the effector devices, and measuring effector responses to monitor improvement of breathing. Functional domains for breathing include but are not limited to lung function, brain function, heart function (FIGS. 2,3,6). Sensed signatures include, but are not limited to, sound produced by breathing, airflow from the pharynx, chest movement (excursion) in breathing, neck muscle movement in breathing, skin oxygenation from breathing, CO2 content of the skin from not breathing, optical reflectance of the skin, heart rate, variability in heart rate, sympathetic nerve activity (during obstruction or anxious breathing), central nervous system activation (EEG), muscular activity, involuntary movement such as gasping or moving limbs, and all components of a polysomnogram test that include brain (EEG), eye movements (EOG), muscle activity or skeletal muscle activation (EMG), heart rhythm (ECG) respiratory airflow and respiratory effort and peripheral pulse oximetry.

In another aspect, there is provided a method and system for treating sleep disordered breathing, the method including selecting one or more functional domains associated with breathing during sleep, applying effector signals to effector devices to modulate said functional domains to treat sleep disordered breathing and measuring effector responses to monitor improvement in sleep breathing. Functional domains for sleep disordered breathing include but are not limited to brain function, central and peripheral nervous system function, lung function, heart function, endocrine function. Effector signals for sleep disordered breathing include, but are not limited to, modulation of sleep cycles by light, electrical, auditory or heating stimuli, modulation of chest movement by stimulation of nerves or muscles of breathing, modulation of neck and pharyngeal muscles by electrical stimulation to prevent obstruction.

In another aspect, there is provided a method and system for treating breathing disorders, the method including selecting one or more functional domains associated with breathing, applying effector signals to effector devices to modulate said functional domains to treat disordered breathing and measuring effector responses to verify improvement in breathing. Functional domains for breathing disorders include but are not limited to brain function, central and peripheral nervous system function, lung function, heart function, endocrine function. Effector signals for breathing disorders include, but are not limited to, modulation of alertness cycles by light, electrical, auditory or heating stimuli, modulation of chest movement by stimulation of nerves or muscles of breathing, modulation of neck and pharyngeal muscles by electrical stimulation to prevent obstruction, increasing inspiratory depth using devices, and amelioration of bronchospasm by appropriate medications.

In another aspect, there is provided a method and system for treating central sleep apnea, the method including identifying an effector organ or system from one or more local areas of the head and neck (the effector region being functionally associated with one or more functional domains that control sleep, e.g. brain), and delivering a therapeutically effective amount of energy to stimulate the effector to treat the central sleep apnea, while minimizing stimulation of other regions of the body. Energy can be electrical energy to the body including periphery or scalp, thermal energy to various regions of the body, light stimulus to be sensed by the eyes, vibratory stimuli to various regions of the body.

In another aspect, there is provided a method and system for modulating mental function, the mental function including one or more of alertness, cognition, memory, mood, attention and awareness, the method including selecting one or more functional domains associated with mental function, monitoring sensed signatures from said functional domains and applying stimulation at one or more effector devices to modulate mental function. Functional domains associated with mental function include a brain domain with sensed signatures including the scalp EEG, scalp temperature; a central and peripheral nervous domain with sensed signatures including sympathetic nerve system activity, peripheral nerve activity; a skin domain with sensed signatures including pilierection (hairs standing up); a heart domain with sensed signatures including heart rate, pulse volume, cardiac contractility measures; a lung domain with sensed signatures including breathing rate, breathing depth, oxygenation; an eye domain with sensed signatures including pupillary diameter, pupillary fluctuations, scleral color; an endocrine domain with sensed signatures of thyroid or adrenocortical systems; a musculoskeletal domain with sensed signatures including muscle tone, muscle oscillations, muscle response to stimuli (reactivity), and others. Effector responses can modulate mental function using consumer devices or other devices to modulate these functional domains.

In another aspect, there is provided a method and system for modulating mental function, the method including identifying a target region selected from localized areas of the body (the target region being functionally associated with parts of the brain that govern the mental function), the mental function including one or more of alertness, cognition, memory, mood, attention and awareness, and delivering a therapeutically effective amount of energy to stimulate the target region to modulate the mental function, while minimizing stimulation of other regions of the body. Energy can be electrical energy to the body including periphery or scalp, thermal energy to various regions of the body, light stimulus to be sensed by the eyes, vibratory stimuli to various regions of the body.

In another aspect, there is provided a system for interacting with the human body, the system including a processor and a memory storing instructions that, when executed by the processor, performs operations including detecting bodily signals associated with one or more bodily functions at one or more sensors associated with the human body, processing the bodily signals to create one or more sensed signatures, processing the signatures using an enciphered functional network to determine one or more effector responses needed to control a bodily task, delivering one or more effector signals, monitoring one or more effector responses, and controlling a bodily task.

In another aspect, there is provided a system to enhance performance of one or more tasks, the system including a processor and a memory storing instructions that, when executed by the processor, performs operations including detecting signals associated with the task at one or more sensors, processing the signals to create one or more sensed signatures, processing the signatures using an enciphered functional network, determining one or more effector responses needed to enhance performance of the bodily task, delivering one or more effector signals (the effector signals based on the one or more effector responses), and enhancing performance of the task. Delivering the one or more effector signals may be performed using the enciphered functional network.

In another aspect, there is provided a system to treat a disease, the system including a processor and a memory storing instructions that, when executed by the processor, performs operations including detecting signals at one or more sensors associated with one or more specific bodily tasks, processing the signals to create one or more sensed signatures of the one or more bodily functions, processing the signatures using an enciphered functional network to determine one or more effector responses needed to treat a disease, delivering one or more effector signals (the effector signals based on the one or more effector responses), and treating the disease. Delivering the one or more effector signals may be performed using the enciphered functional network.

In another aspect, there is provided a system to transform nerve activity associated with one or more bodily functions, the system including a processor and a memory storing instructions that, when executed by the processor, performs operations including detecting signals of nerve activity associated with the one or more bodily functions at one or more sensors, processing the signals to create one or more sensed signatures associated with one or more functional domains, processing the signatures using an enciphered functional network to transform nerve activity, delivering one or more effector signals (the effector signals based on the one or more effector responses), and transforming nerve activity. Effector signals may be delivered via the enciphered functional network.

In another aspect, there is provided a system to control a device using biological signals, the system including a processor and a memory storing instructions that, when executed by the processor, performs operations including detecting signals using one or more sensors, processing the signals to create one or more sensed signatures, assigning said sensed signatures to one or more functional domains, processing the sensed signatures from one or more functional domains using an enciphered functional network to determine one or more effector responses to control the device, delivering one or more effector signals (the effector signals based on the one or more effector responses), and controlling the device. Effector signals may be delivered via the enciphered functional network.

In another aspect, there is provided a system to measure visual function in an animal, the system including a processor and a memory storing instructions that, when executed by the processor, performs operations including detecting bodily signals associated with sensory activation, processing the bodily signals to create one or more sensed signatures representing quantitative measures of sensation, assigning said sensed signatures to one or more functional domains of sensation, and processing the sensed signatures using an enciphered functional network to determine one or more effector responses needed to enhance the bodily function of the animal. Effector signals may be delivered via the enciphered functional network.

In another aspect, there is provided a system for improving a specific human performance, the system including a processor and a memory storing instructions that, when executed by the processor, performs operations including identifying one or more regions of a human body associated with parts of the brain that serve a specific function, placing low energy stimulating electrodes proximate to the one or more regions of the human body, applying stimulation through the electrodes to activate the parts of the brain, and measuring changes related to the parts of the brain to verify improvement of the specific human performance.

In another aspect, there is provided a system for improving a performance of a specific human task, the system including a processor and a memory storing instructions that, when executed by the processor, performs operations including identifying one or more functional domains associated with that specific human task, using consumer or medical devices to modulate one or more functional domains, and measuring sensed signatures to monitor changes in performance of the specific task.

In another aspect, there is provided a system for treating a sleep disorder, the system including a processor and a memory storing instructions that, when executed by the processor, performs operations including selecting one or more regions of a patient's central nervous system and/or peripheral nervous system associated with sleep functioning, and applying low energy stimulation through electrodes to activate the patient's one or more regions of central nervous system and/or peripheral nervous system to treat the sleep disorder.

In another aspect, there is provided a system for treating a sleep disorder, the system including a processor and a memory storing instructions that, when executed by the processor, performs operations including selecting one or more functional domains associated with sleep functioning, and using consumer or medical devices to modulate said one or more functional domains of a sleep disorder, and measuring sensed signatures to treat the sleep disorder.

In another aspect, there is provided a system to modulate mental function, the system including a processor and a memory storing instructions that, when executed by the processor, performs operations including identifying a target region selected from localized areas of the body (the target region being functionally associated with parts of the brain that govern the mental function, including one or more of alertness, cognition, memory, mood, attention and awareness), and delivering a therapeutically effective amount of energy to stimulate the target region to modulate the mental function, while minimizing stimulation of other regions of the body.

In another aspect, there is provided a system for treating abnormal mental function, the system including a processor and a memory storing instructions that, when executed by the processor, performs operations including selecting one or more functional domains associated with mental function, using consumer or medical devices to modulate said one or more functional domains of mental function, and measuring sensed signatures to treat abnormal mental function. Functional domains for mental functioning include but are not limited to brain function including sensed signatures of the EEG, function of the central and peripheral nervous system function including sensed signatures of peripheral nerve firing in patient specific regions of the body, lung function including sensed signatures of breathing rate, regularity and oxygenation, the ocular system including sensed signatures of pupillary diameter and reactivity to light, endocrine function including changes in body chemistry and release of hormones, and heart function including sensed signatures of heart rate and pulse volume.

In another aspect, there is provided a system to enhance attention, the system including a processor and a memory storing instructions that, when executed by the processor, performs operations including selecting one or more regions of a patient's central nervous system and/or peripheral nervous system associated with an attention disorder, and applying low energy stimulation through electrodes to activate parts of a patient's central nervous system and/or peripheral nervous system to treat the attention disorder.

In another aspect, there is provided a system for treating attention disorders, the system including a processor and a memory storing instructions that, when executed by the processor, performs operations including selecting one or more functional domains associated with attention disorder, using consumer or medical devices to modulate said one or more functional domains of attention disorder, and measuring sensed signatures to treat attention disorder.

In another aspect, there is provided a system to treat obstructive sleep apnea, the system including a processor and a memory storing instructions that, when executed by the processor, performs operations including identifying a target region from one or more local areas of the head and neck (the target region being functionally associated with one or more parts of the brain that control sleep), and delivering a therapeutically effective amount of energy to stimulate the target region to treat the obstructive sleep apnea, while minimizing stimulation of other regions of the body.

In another aspect, there is provided a system for treating obstructive sleep apnea, the system including a processor and a memory storing instructions that, when executed by the processor, performs operations including selecting one or more functional domains associated with obstructive sleep apnea, using consumer or medical devices to modulate said one or more functional domains of obstructive sleep apnea, and measuring sensed signatures to treat obstructive sleep apnea. Functional domains for obstructive sleep apnea include but are not limited to brain, central and peripheral nervous system, lung, heart, endocrine. Sensed signatures of the brain domain for sleep include, but are not limited to, scalp electrical signals and EEG, scalp temperature. Sensed signatures of the peripheral nerve domain for sleep include, but are not limited to, rates and patterns of peripheral nerve firing, rates and patterns of pharyngeal muscle nerve firing, rates and patterns of phrenic nerve activity. Sensed signatures of the lung domain for sleep include, but are not limited to, sound produced by sleep-breathing (e.g. normal breaths snoring), chest movement rate and depth. Sensed signatures of the peripheral muscular domain for sleep include, but are not limited to, body movement on external motion sensors. Sensed signatures of the skin domain for sleep include, but are not limited to, skin oxygenation patterns, regional temperature in the face/torso/periphery; regional skin impedance in the face/torso/periphery; regional chemical composition (sodium, others) in the face/torso/periphery. Sensed signatures of the heart domain for sleep include, but are not limited to, heart rates and variability in heart rate during sleep. Components of the polysomnogram during sleep can also be sensed signatures and include brain (EEG), eye movements (EOG), muscle activity or skeletal muscle activation (EMG), heart rhythm (ECG) respiratory airflow and respiratory effort and peripheral pulse oximetry. Effector signals for obstructive sleep apnea include, but are not limited to, modulation of sleep cycles by light, electrical, heating or auditory stimuli, modulation of breathing by stimulation of nerves or muscles of the pharynx or of breathing, modulation of peripheral muscles by heating or electrical stimulation.

In another aspect, there is provided a system to treat central sleep apnea, the system including a processor and a memory storing instructions that, when executed by the processor, performs operations including identifying a target region from one or more local areas of the head and neck (the target region being functionally associated with one or more parts of the brain that control sleep), and delivering a therapeutically effective amount of energy to stimulate the target region to treat the central sleep apnea, while minimizing stimulation of other regions of the body.

In another aspect, there is provided a system for treating central sleep apnea, the system including a processor and a memory storing instructions that, when executed by the processor, performs operations including selecting one or more functional domains associated with central sleep apnea, using consumer or medical devices to modulate the one or more functional domains of central sleep apnea, and measuring sensed signatures to treat central sleep apnea. Functional domains for sleep include but are not limited to brain function, central and peripheral nervous system function, lung function, heart function. Effector signals for sleep include, but are not limited to, modulation of sleep cycles by light, electrical, heating or auditory stimuli, modulation of breathing by stimulation of nerves or muscles, modulation of peripheral muscles by heating or electrical stimulation

One motivation for this invention is that detailed mechanistic solutions for the therapy of many complex bodily functions are often unavailable. This reflects several factors. First, there is inter-individual variation in regions of control—for instance, the biological neural network for speech may differ from one person to another. For functions with a nervous component, this may represent the unique fashion in which higher cognitive functions and memories are shaped during growth and development or genetically established in each person. Secondly, many functions are plastic—changes in the environment or disease can alter control regions or responses. Changes can be gradual or abrupt, causing variations over years, months or even weeks that may reflect normal development, aging or dysfunction. This may explain why therapies that are initially effective can become ineffective over time. Thirdly, our conceptual knowledge of functional domains in the central and peripheral nervous system is in its infancy. Analogous arguments can readily be made for limitations in our conceptual knowledge of functional domains in other bodily or organ systems. It is thus a major challenge to understand or modulate a bodily function using the classical paradigm of defining specific target region(s) of control for that function, then modulating it to alter bodily function.

Several innovations separate this invention from the prior art. First, the invention creates an enciphered network for the bodily task and/or function. This represents the bodily function as network of functional domains, each comprised of sensed signatures and effector responses. Functional domains can span several organ systems and are associated with that bodily function, yet their mechanistic relationship may yet not be fully delineated. Second, the present invention is patient-tailored. Sensed signatures and effectors are identified though sensed signals for each individual, and do not rely upon defined mechanistic pathways. This core aspect of the invention was designed because the same functional domain often has different manifestations in each individual, although traditional devices apply a ‘one size fits all’ sensed system to each individual. Signatures can be nervous or non-nervous system related. Third, diagnosis or therapy is inherently adaptive, such that a similar abnormality may produce different signatures and/or require different effector signals at distinct periods in time, or under different conditions, in the same individual or between individuals. The feedback between sensed signatures, the enciphered network and effector responses adapts using various processes including simple feedback loops, database comparisons to other individuals or populations, manual human reprogramming, or machine learning. Fourth, certain embodiments of the device combine biological and non-biologic devices, together or individually. The enciphered representation can accommodate additional signatures over time, that can be extrinsic or artificial signals as well as biological ones. Therapy can ultimately be delivered by an external device and/or by direct stimulation or suppression of an effector. Embodiments include improvements of sleep apnea (as well as other sleep disorders (e.g. insomnia) and breathing disorders), the body's response to heart failure including fluid gain, obesity or weight management, alertness, sleep, memory and mental performance or cognition.

In a preferred embodiment of the invention, signatures are sensed on a repeated and even near-continuous basis. This can be accomplished by consumer or medical grade sensors. In this embodiment, signatures during defined ‘health’ for that function constitute a tailored baseline for that individual. These sensed signatures can be used to train/calibrate the enciphered functional network. In this embodiment, subsequent sensed signatures which deviate beyond an individual limit from the ‘health’ state indicate abnormal functioning in that individual. It is important to note that this signature may have a different meaning in another individual, or in that individual under different conditions (e.g. sleep versus awake, sedentary vs exercise). This is fundamentally different from the prior art, in which a ‘population’ range for normal and disease are applied across multiple patients with little scope to tailor them to the individual. This aspect of the invention enables “personalized medicine” or “precision medicine” using a computerized approach.

The core aspect of the invention of functional domains for a task, measured from or stimulating an interconnected region of the network that may be neural, vascular or other, is novel at several levels and has not been addressed by devices in the prior art. One example way to better understand this concept is by considering the disease of sleep apnea which may be central or obstructive.

Functional domains for central sleep apnea in this invention include sensed signatures of brain function (measurable on the EEG), reduced oxygenation levels and increased carbon monoxide levels in the blood (measurable from skin sensors), increased heart rate and altered patterns of heart rate, altered nasal and/or oral airflow (measurable from airflow sensors or sensors detecting changes in the auditory signature of breath sounds), and other signatures. Observed signatures in individuals, which may be embodied in the invention although are not fully defined mechanistically, include nocturnal rostral fluid shift from the legs (that may link central sleep apnea with heart failure). Similarly, effector responses for central sleep apnea include nerve function and muscles in the tongue, oropharynx, neck, diaphragm, intercostal muscles and accessory muscles (measurable by nerve firing rates). The present invention will use these sensed signatures of brain or nerve activity, chest wall movement, bioimpedance at the skin (to assess for a rostral change indicative of fluid shift), or oxygenation for diagnosis and monitoring. In an embodiment for treatment, the invention may result in varying effector responses.

Functional domains for obstructive sleep apnea include sensed signatures of brain function (measurable on the EEG), central and peripheral nervous system (measureable by nerve firing rates and periodicity), oxygenation and carbon monoxide levels in the blood (measurable from skin sensors), chest wall movement (measurable by chest wall excursion or muscle activity), neck and pharyngeal muscles (measurable by increased tone at times of obstruction), altered heart rate and patterns (variability) in heart rate, altered nasal and/or oral airflow (measurable from airflow sensors or sensors detecting changes in the auditory signature of breath sounds), and other less defined functions. Effector responses for obstructive sleep apnea include light, heat, auditory and electrical stimulation to alter sleep/awake cycling, electrical stimulation to alter neck or pharyngeal muscle tone, stimulation of the diaphragm and intercostal muscles.

Other sensed signatures for breathing activity are measured as rate, periodicity and depth. In one preferred embodiment, the sensor detects chest wall movement which can measure breathing rate, depth and periodicity. In another preferred embodiment, the sensor detects fluctuating levels of oxygenation directly, chemically or using optical measures of oxygenated hemoglobin. In yet another embodiment, the sensor may detect heart rate changes with breathing (i.e. sinus arrhythmia). In yet another embodiment, the sensor detects altered nasal and/or oral airflow. In yet another embodiment, the sensor detects changes in the auditory signature of breath sounds. Placement of the sensor on the nose, mouth, chest, neck, abdomen, or other locations where an auditory signal can be sensed can indicate specific breathing functions or disorders. For example, exaggerated breath movement in the neck but minimal movements in the chest are typical of obstructive apnea. Minimal movement on both the neck and chest may indicate central hypopnea and/or central apnea. Exaggerated breathing at high rate may indicate higher metabolic activity, anxiety, exercise or such states. Other sensors can be located in positioned familiar to one skilled in the art.

Chest wall sensors can detect displacement of a single sensor, relative displacement of 2 or more sensors, vibration, measures of volume or measures of electrical impedance. In this invention, the sensed signature of abnormal chest wall impedance includes a ratio of lower body impedance (e.g., leg, lower back) to higher body impedance (neck and chest)—i.e., higher impedance in lower body (less extracellular water), lower impedance in upper body (more extracellular water). This could also be expressed as upper-to-lower body conductance. This could also include measuring impedance to different forms, patterns, or waveforms of electrical energy.

Another sensed signature for various domains is nerve activity, measured by the rate and periodicity of nerve firing, circadian rhythms, the type of nerve firing, and their spatial distribution. For the enciphered functional network in this invention, a preferred embodiment uses skin electrodes to obtain sensed nerve signals. This differs from traditional measures of nerve activity, e.g. the electroneurogram (ENG) by placing an electrode in neural tissue. This invasive approach is less well suited to continuous recordings or consumer applications. Skin detection is already used in the EEG (electroencephalogram), which is a form of electroneurogram which uses several electrodes around the head to record general activity of the brain. The resolution of skin electrodes is sufficient to detect signals and create sensed nerve signatures, with nerve firing rates, types and distributions analyzed by the invention. In another preferred embodiment, sensors measure subtle changes in reflectance or emission of electromagnetic radiation from nerve activity including infrared (heat). In another preferred embodiment, sensors measure electrical resistance changes from nerve activity. Sensors can be placed in different skin regions, e.g. near neck or chest muscles to measure nerve activity related to breathing, on the head to measure alertness, on the limbs to measure nerve activity related to muscles on those limbs and other locations familiar to one skilled in the art.

Non-invasive sensors in the invention can serve as surrogates for the electroneurogram (ENG). In the ENG, electrical activity generated by neurons is recorded by the electrode and transmitted to an acquisition system, which allows visualization of activity of the neuron. Each vertical line in an electroneurogram represents one neuronal action potential. Depending on the precision of the electrode used to record neural activity, an electroneurogram can contain the activity of a single neuron to thousands of neurons, Researchers adapt the precision of their electrode to either focus on the activity of a single neuron or the general activity of a group of neurons, both strategies having their advantages depending upon the application. In this invention, patterns of non-invasive sensed nerve signatures over time are used to indicate ENG changes over time in an individual during normal and abnormal states of a bodily function.

Non-invasive sensors in the invention can serve as surrogates for the electromyogram (EMG). In the EMG, electrical activity generated by muscle cells is recorded by the electrode and transmitted to an acquisition system, which allows visualization of activity of muscular tissue. Vertical lines in an EMG represents one or more muscle units. Depending on the precision of the electrode used, an EMG can contain the activity of single to thousands of muscle units. Researchers adapt the precision of their electrode to either focus on the activity of smaller or larger muscle regions, both strategies having their advantages depending on the application. In this invention, patterns of non-invasive sensed muscle signatures over time are used to indicate EMG changes over time in an individual during normal and abnormal states of a bodily function.

Other sensed signatures for the task of sleep include, vasodilation during sleep, reduced electrical resistance in the skin from altered electrolytes or water accumulation as part of the body's response to heart failure or sleep-breathing disorders, altered skin absorption or emission of components of the electromagnetic spectrum including near-infrared due to changes in oxygenation of blood, or carbon dioxide accumulation during heart disorders or breathing disorders, measured alterations to other forms of applied non-electrical energy including optical signals (altered reflectance), sound or ultrasound (different sonic reflectance and scattering), and potentially altered spectroscopic signals of body chemistry that can be sensed.

In one preferred embodiment, the enciphered functional network uses machine learning to associate sensed signatures with normal breathing. In one such embodiment, an artificial neural network is used, which comprises 3 typical elements:

1. The connection pattern between different layers of nodes (artificial neurons): Nodes are typically represented as networks, and there may be variations in the number of layers and the number of nodes per layer in the input, hidden (internal) and output layers. Nodes can be connected to all nodes in layers above and below, but differential connections can also be implemented;

2. Connections weights between nodes, i.e. interconnections, which are updated in the process of learning;

3. The mathematical activation function: determining how the weighted input of each node is converted to its output. Typically, the activation function ƒ(x) is a composite of other functions g(x), which can in turn be expressed as a composite of other functions. A non-linear weighted sum may be used, i.e. ƒ(x)=K (Ξᵢ wᵢgᵢ (x), where K (the activation function) may be sigmoidal, hyperbolic or other function.

A variety of connection patterns, weight and mathematical activation functions can be selected, and a variety of updating functions are possible for any embodiment. Specific forms are optimal for different specific enciphered networks. For example, the enciphered network linking sound analysis with sleep disordered breathing will be less complex than the network for cognitive function or alertness. However, extending the enciphered functional network for sleep disordered breathing to include movement, heart rate fluctuations, changes in skin oxygen, changes in skin resistance (reflecting sympathetic nervous system activation) and changes in the other neural patterns (e.g. the EEG) will be more complicated. Recent approaches to complex tasks such as handwriting analysis and speech recognition use recurrent neural networks, in which node interconnections form a directed cycle to enable dynamic temporal behavior. Recurrent networks have an ability to process arbitrary sequences of inputs, which differs from designs such as feedforward networks and may enable them better suited to complex tasks.

Alternative forms of adaptation of the enciphered network may use rule-based algorithms in the “if-then-else” formulation, heuristics, or other patterned associations to link sensed signatures with behaviors for an individual. Several other forms of machine learning can be applied, and will be apparent to an individual skilled in the art.

In a preferred embodiment, machine learning is applied to define patterns of sensed signatures over time associated with normal breathing, that include circadian variations for that individual. Deviations from normal breathing for that individual can then be identified by deviations from these learned patterns. If abnormal breathing such as apnea (i.e. pauses in breathing) arises during sleep, the invention is capable of applying effector responses to alleviate sleep apnea that are tailored to the individual, e.g. to alter activity of the functional domains associated with sleep-disordered breathing. In these examples, the machine learning is trained using iterative analyses of when the individual is at times of low breathing-health and when the individual is at times of high breathing-health. The response to therapy (i.e., effector response) can be assessed repeatedly from sensed signatures, and therapy can be withdrawn or continued based upon these signatures. This differs from the prior art in which therapies such as continuous positive airway pressure or nerve stimulation are often delivered empirically, continuously or in predetermined fashions without the ability to tailor therapy adaptively to physiological indexes in that individual. This invention provides physiological indexes for that individual.

Creating and defining a network of functional domains is a unique approach for interfacing with bodily functions. For instance, a patient with heart pain (angina pectoris) or a heart attack (myocardial infarction) often experiences “radiated pain” to the left arm, shoulder or other regions. Some patients experience only arm pain from cardiac ischemia—i.e., arm pain is a sensed signature for those specific patients. This signature may not be relevant to other individuals a priori—but can be learned by the enciphered network for that individual. In this way, the invention can now detect nerve activity in the arm below the typical nerve firing rates for sensed “pain”, providing the device with an early warning sensor for heart pain (“angina”) to provide therapy or alert medical personnel.

In another example, patients with problems of the abdominal viscera (stomach, small intestine, large intestine) that may include normal “indigestion” as well as diseases often experience vague discomfort on the abdominal wall through imprecisely defined and variable visceral and somatic nerves. Massaging this region is an example of counter-stimulation (competitive stimulation) that can alleviate the visceral organ pain. In one embodiment, the invention will thus provide algorithmically determined vibratory stimulation to appropriate skin regions within the “functional domain” of the bodily function to alleviate pain. In another embodiment, the invention will provide heat (thermal stimulation) as counter irritation. In yet another embodiment, the competitive stimulus will be delivered at sensory input regions which compete functionally with the sensory input regions for pain.

As yet another example, nerve firing in cutaneous or other accessible nerves (e.g., mucous membranes of mouth, anus, or skin of the external auditory meatus) may share neural control regions with other organs, such as heart pain or even abnormal heart rhythms. Effector signals can be delivered to specific regions of the functional domain to alleviate heart pain or other abnormalities. Other components of a functional domain may include blood vessel flow, vasomotor reactivity, skin electrical conductivity, heart rate or heart rate variations, breathing rate, cellular edema and other indices illustrated throughout the specification.

Therapy is individually tailored and not empirically delivered. Baseline signatures such as rates and patterns of nerve firing during a desired level of functioning are analyzed and learned in each individual and may be combined with other signatures within the enciphered functional network. In states such as sleep-disordered breathing, heart failure, fatigue and others, fluctuations outside this normal range are detected and can be used to monitor disease or performance. Therapy such as stimulating neck muscles for obstructive sleep apnea, stimulating accessory muscles or alertness centers for central apnea, or therapy for heart failure and other conditions can be monitored (e.g., by effector response) and tailored to machine learned signatures. Functionality can thus be modulated without direct knowledge or access to the primary physiological target and without detailed pathophysiological knowledge of that function.

Nerve signatures may be shared between many functions, e.g., based on dermatomal distributions of peripheral nerves. One example is sensation of the tip of shoulder blade at the “C234” region, control of deltoid muscle function by the “C56” region, and control of the diaphragm muscles and hence breathing at the “C345” region. Thus, sensation at the shoulder can indicate shoulder stimulation, or pain in portions of the heart adjacent to the diaphragm. Stimulation at these regions by direct electrical stimulation, vibratory stimuli, heat or other can produce a competitive stimulus to the measured function.

Brain signatures can be assessed directly via the EEG or simplified EEG measured from the scalp by many types of electrical sensor. For instance, scalp activity in the alpha (7.5-12.5 Hz), beta (12.5-30 Hz) or gamma (25-40 Hz) bands indicate states of awakeness (wakefulness) or heightened or alertness; activity in the delta (0.1-3 Hz) or theta (4-7 Hz) bands indicate drowsy (or comatose) states. Depending on sensed activity, interventions can be applied to the scalp or other domains of the network while monitoring alpha, beta or gamma signatures to facilitate alertness. In each case, the invention is novel in that it derives patient-tailored signatures for a given function using machine learning, and will apply interventions algorithmically in a tailored feedback loop. In one preferred embodiment this will enhance sleep function.

Peripheral nerve signatures are numerous and varied. For instance, increased nerve firing of the cervical sympathetic plexus in the head and neck may be associated with alertness or rapid eye movement (REM) sleep, and reduced activity may be associated with drowsiness or stages I-IV of sleep. Stimulation of those regions of the head and neck can be used to increase alertness. Increased firing of the accessory (XI), facial (VII) or other cranial nerves may indicate impending obstructive sleep apnea, and may provide targets for therapy.

This invention adapts to concepts of neural plasticity. Plasticity refers to alterations in the pathways of nerves and connections (synapses) from changes in behavior, environment, neural processes, thinking, and emotions, and also to changes resulting from injury. This concept has replaced prior teachings that the brain and nervous systems are static organs. New studies show that the brain changes in anatomy (structure) and physiology (functioning) over time. There are several examples, e.g., DARPA limb projects, stroke victims recovering function after months or years of physical or occupational therapy despite having infarcted the traditional brain areas for the target function. Plasticity is also observed in peripheral nerves, for instance the distribution of a functioning nerve (dermatome) can expand into an adjacent distribution of diseased nerve supply. In other words, a singular function can be assumed or subsumed by different regions of the central or peripheral nervous system, that will also have non-neural implications, e.g., on measured blood flow, galvanic skin resistance or other physiological parameters.

There are several non-nerve domain signatures. For instance, deoxygenation of hemoglobin noted via an oxygen sensor on the skin of a finger (via optical reflectance or plethysmography) can indicate hypopnea or apnea. Increased skin temperature or blood flow (absorption in red wavelengths on an optical sensor) may occur in stages I-IV sleep from parasympathetic activation. Novel skin sensors can detect changes in biomarkers such as glucose (to detect diabetic states, need to eat), INR (a test of blood clotting ability for some patients on blood thinners) and a new generation of sensors for drugs in the blood stream, chemical changes on the skin and so on, Interpretation of these signatures can be troublesome but is linked in this invention by machine learning to a specific function, e.g., fever increases skin temperature, but is accompanied by increased breathing rate and altered skin biochemistry/impedance (due to perspiration). By learning based on multiple signatures, temperature information can be used in this case to distinguish changes in breathing rates due to fever from that due to central sleep apnea.

This invention uses the core principle that continuous machine learning will enable its functionality to be retained even when plasticity occurs, i.e. when the task for an individual is mediated by different proportions of physiological functions overtime, again using sensed signatures in that individual without the need for precise physiological mapping knowledge for that function. For instance, in classical Pavlovian training, dogs were trained to salivate when exposed to non-food-related stimuli that had previously been associated with food in training. In other words, a new trained stimulus—functional interaction—was used without knowledge of detailed physiological linking for that function.

This invention also encompasses personalized learned feedback loops, to modulate a desired bodily function by algorithmic machine learning analogous to classical Pavlovian conditioning. In a training mode, stimulation is applied during normal periods—for instance, vibratory stimulation of the skin of the lower back on days of anticipated restful sleep. Subsequently, if sleep is interrupted, trained modes of stimulation are applied. This mode can be applied to various bodily functions including but not limited to alertness, memory, sleep and sleep-disordered breathing.

The present invention identifies functional domains empirically, and provides computational customized, individualized solutions. This differs from the prior art in which, for example, a preferred embodiment for sleep disordered breathing may stimulate cranial nerves (e.g., trigeminal or hypoglossal), but through unclear mechanisms that may in fact inadvertently work by training certain responses or stimulating other regions than those intended.

In another set of preferred embodiments, the enciphered network can be used to enhance body performance in non-disease states. One direction is to utilize unused body capacity. During office work, for instance, humans often underuse natural sensors or effectors on the torso, leg and arm yet more frequently use sensors/effectors on the face (eye, mouth) and hands. Stimulation of underused regions by a device can extend the sensory capacity (bandwidth) of an individual. When combined with artificial sensors, these underused regions can also be used to provide a “sixth sense” (see drawings) to extend sensation to biologically unsensed stimuli (e.g., a carbon monoxide sensor can provide vibratory stimuli to unused portions of the body), to train the body (e.g., improve alertness) or other function.

Enhancement of performance may require specific stimulation patterns that vary based on frequencies, amplitudes and sites of stimulation. This information can be derived by machine learning of sensed signatures or patterns in each individual. Another approach is to use patterns from individuals who are highly functioning in that desired modality—from a de-identified database, by crowd sourced data collection from wearable devices or by other means. These patterns can then serve as inputs for the enciphered network, that will interface them to the symbolic representation for an individual to tailor them appropriately.

Effector stimulation could avoid inadvertent recruitment of existing bodily functions by applying non-physiological or atypical physiological stimuli. This can be achieved by using neural frequencies or patterns that are not part of normal processing or pathways, such as outside the normal sensed frequency, or with a different pattern, or at a different (lower) amplitude. Using other examples in this disclosure, the invention may detect subclinical nerve firing in the functional domain for cardiac ischemia as an early warning for angina, or application of subclinical amplitudes of nerve stimulation to the accessory muscles to stimulate breathing (for central sleep apnea) or neck (to improve alertness). These safeguards will avoid invoking behavioral change, sensation by the brain and/or changing memory of an event (Redondo et al., Nature 2014).The invention can work with several types of sensors individually or in combination. Examples include solid physical sensors such as FINE (singularityhub.com/2013/07/24/darpas-brain-controlled-prosthetic-arm-and-a-bionic-hand-that-can-touch/), traditional ECG- or EEG-electrical sensors, non-solid sensors such as electrostatic creams, sensors for bioimpedance, piezoelectric film sensors, printed circuit sensors, photosensitive film, thermosensitive film, and external-oriented sensors not in contact with the body such as video, IR, temperature, gas sensors, as well as other sensors. Various embodiments of the invention use novel sensors, such as skin sensors to detect glucose, drug concentrations or other chemical agents. In general, sensors detect stimuli and transduce the information through a constructed/created (non standard or non-somatotopic) path to active nerves.

Processing elements include a digital signal processor to interface with output elements that can stimulate different parts/nerves of the body, or cause mechanical action in an external machine. Such elements could include traditional computing machines with integrated circuits in isolation or networked (e.g., cloud computing), biological computing, integrated biological/artificial devices (cybernetic) or utilizing unused biological capacity to perform specific, directed tasks. One potential embodiment is to use unused computational capacity of the central nervous system to perform pattern recognition in lieu of programming an artificial computer for this purpose. This can be accomplished by training an individual to recognize a visual/auditory/olfactory or other sensation and then sensing the sensed signature of that evoked response when that stimulus is subsequently encountered.

Effector elements can include direct electrical outputs, piezoelectrical devices, visual/infrared or other stimulatory systems, nerve stimulating electrodes or servo motors to control a limb, digitized electronic signals such as radiofrequency or infrared transmissions, or even virtualized data such as avatars in a virtual world interface or elements in a large database that can be queried, as well as other effector elements now existing or yet to be developed.

Applications of effector elements can be for diagnostic purposes such as detecting stimuli or body functions (e.g., visual function, visual disease progression, mood, alertness, detecting injury such as traumatic brain injury, cardiac electrical and/or mechanical function, subclinical seizure detection), detecting external world situations or environments without subjecting the human body to discomfort (e.g., sensing heat in a fire, detecting oxygen or toxic gas content in the external environment such as a mine).

Effectors can be applied for medically related therapy such as brain related function (e.g., brain stimulation for patients with sleep disorders or central apnea, biofeedback for stroke rehabilitation, deep brain stimulation for motion or seizure disorders), other neurological diseases (e.g., substituting artificial sensor data in patients with peripheral neuropathy, cortical blindness, congenital deafness, biofeedback stimulation of muscles), cardiac disease (e.g., arrhythmias treated with implanted devices, cardiac function improved with mechanical or electrical devices), response to obesity, or other organ disease modified with directed electrical or mechanical elements.

Applications of machine learned therapy using this invention can be for training, learning and performing of physiological activities or mechanical, non-physiologic functions. Unlike the prior art that applies non-specific stimulation, e.g., transcranial direct current stimulation (ref: www.scientificamerican.com/article/amping-up-brain-function), the present invention can sense, machine learn, optimize, and then deliver specific therapy modulated via a feedback loop. This will provide tailored therapy to modify many complex functions.

Other applications for this invention include improving athletic performance after injury (e.g., direct stimulation to muscles to regain lost function, biofeedback to maintain heart rate within desired range during controlled exercise, brain stimulation), enhancing sensory perceptions (e.g., artificial visual sensors for facial recognition, artificial auditory sensors to detect previously inaudible information), performing tasks in non-typical ways by overcoming constraints or developing more efficient solutions (e.g., driving a car with small finger movements or eye motion amplified by artificial device, controlling an external device biologically, e.g., small eye or limb movements to control a computer interface). Examples of mechanical functions include biological operation of a mechanical exoskeleton for soldiers, performing tasks too difficult or dangerous for humans such as deep sea exploration, armed combat, or basic tasks such as controlling a computer, video games or remote controls.

In summary, the invention incorporates a combined biological-artificial network, referred to as enciphered functional network (or representation), to modulate specific tasks (such as complex bodily functions often requiring brain or nerve involvement, or higher cortical functions). Sensors (biological or artificial) sense the activity of the measured task. This sensed activity is enciphered as sensed signatures for a specific task, then a series of algorithms including but not limited to machine learning and specific hardware components modulate the network using biological, artificial or hybrid effectors (e.g., stimulating electrodes). The network can directly augment a function (e.g., sleep), or form a new function via existing elements (“retasking” a function, e.g., associating lower back stimulation with sleep).

The enciphered network can operate using a symbolic representation specific to each task. Specific representations of each task may be important because the pattern, frequency, and amplitude of stimulation differ considerably between tasks—e.g., modulating electrical activity on the scalp versus the neck or other parts of the body, or stimulating neural elements versus blood vessels.

### BRIEF DESCRIPTION OF THE DRAWINGS

Some embodiments are illustrated by way of example and not limitation in the figures of the accompanying drawings, in which:

FIG. 1 shows a schematic representation of the invention, including biological sensors or external sensors, a signal processing unit and a computing device that can form a representation of bodily functions, e.g., an “enciphered functional network”. A control unit can be used to treat abnormal physiological functions via a device or biological organ (“effector”) tailored by measuring response to therapy in a feedback loop.

FIG. 2 illustrates the invention for one preferred embodiment of breathing health, with functional domain(s) of lung function represented by sensed signatures that can be tracked over time including breath sounds, chest wall movement, movement of the body using sensors in a bed or chair, changes in oxygenation. The enciphered functional network (with analysis engine) combines this analytical system with effector group(s).

FIG. 3 shows a flowchart illustrating how the enciphered functional network represents a bodily function in an individual person, for one preferred embodiment of breathing health, as functional domains represented by sensed signatures. Sensed signatures are analyzed by algorithms that match signature patterns to desired and undesired behavior, to databases (e.g. analyzed using statistical correlation) in a network of “population behavior” or historical behavior of that individual, to monitor function, guide and assess response to therapy.

FIG. 4 shows an example of sensed signatures for a preferred embodiment of breathing health, for functional domains representing nervous system and non-nervous system functions and tasks. The array of sensed signatures becomes the measured representation of that bodily function for that individual person over time.

FIG. 5 shows the task of modifying bodily function using the enciphered network of the invention, here for one preferred embodiment of breathing health. Modification is tailored to the individual via personalized sensory signatures and machine learning in the enciphered network. Modification includes therapy, such as for sleep-disordered breathing, but can also enhance normal function for that individual. Modification operates in a continuous feedback, assessing response via the enciphered network to prevent excessive or deleterious modification.

FIG. 6 shows illustrative body locations for sensed signatures and modifying various functional domains. Sensor locations are indicated by open (white) regions and effector (modifying) regions by filled (black) regions. Their relative size varies in each individual, is determined by machine learning for each individual and is not portrayed to scale.

FIG. 6B shows an illustrative framework for the Enciphered Network. Arrays of sensors or effects connect the invention with the individual person. The processing network links the sensor or effector arrays with health states using logic which can be machine learning, rule-based, heuristic-based, database lookup or other associations.

FIG. 7 shows examples of sensors in this invention, which may comprise a sensor element, power source, microprocessor element, nonvolatile storage and communication element. Several types of sensor element are illustrated, such as photodetector (for skin temperature, metabolic light sensing, drug sensing), galvanometer (for skin impedance), pressure (for weight, skin breakdown), temperature or chemical. The invention can also use external sensors (FIGS. 1, 12-18) that provide a variety of extrinsic or artificial signatures (FIGS. 12-18).

FIG. 7A illustrates consumer sensors that can provide sensed signals for the invention to manage health and disease. This includes a smartphone, which can provide sensed signals of breath sounds (used in one preferred embodiment for breathing health), movement, heart rate and other signals. Other consumer devices include a smartwatch, motion sensor in the house, motion sensor in a bed, chair or automobile or plane seat, consumer microphone, light detector, and weighing scales.

FIG. 7B shows the invention flowchart for managing breathing health and detecting sleep apnea using breath sounds from a smartphone alone, as one preferred embodiment.

FIG. 7C shows an example in which the invention can analyze sounds from a smartphone at distance from the individual to detect normal breaths, snoring and other disturbances. Sound analysis in this test example is validated by reference to a clinical polysomnogram (performed simultaneously with the sound recording), which verifies disturbances. In actual practice, the invention is intended to be used without a polysomnogram.

FIG. 7D illustrates the invention analyzing sounds from a smartphone at a distance from the individual to detect normal breaths, a 20 second period without breathing (apnea), followed by a loud arousal event (sound ‘disturbance’). In this test case, sound analysis is validated by reference to a clinical polysomnogram (performed simultaneously with the sound recording), which verifies disturbances. In actual practice, the invention is intended to be used without a polysomnogram.

FIG. 7E. shows the specific analysis flowchart for analyzing sound files from a smartphone.

FIG. 7F. shows a example in which the invention analyzes sounds from a smartphone alone at a distance from the individual, and detects snoring, periods of no breathing for >10 seconds, and other breath sounds.

FIG. 7G. shows an example in which the invention analyzes sounds from a smartphone alone at a distance from the individual, and detects periods of loud snoring and other breath sounds.

FIG. 7H shows an example in which the invention analyzes sounds from a smartphone alone at a distance from the individual, and detects a period of loud snoring or disturbance/noise using the area under the sound curve.

FIG. 7I shows an example in which the invention analyzes sounds from a smartphone alone at a distance from the individual, and detects a period of noise.

FIG. 7J shows an example in which the invention analyzes sounds from a smartphone alone at a distance from the individual, and detects very low amplitude sound.

FIG. 8 shows some preferred embodiments of sensed signatures of sleep disordered breathing.

FIG. 9 shows a preferred embodiment of effectors to modulate sleep health and treat disease.

FIG. 10 shows some preferred embodiments of sensed signatures for heart failure.

FIG. 11 shows some preferred embodiments of sensed signatures of the body response to obesity.

FIG. 12 shows some preferred embodiments of sensed signatures for other conditions.

FIG. 13 shows one embodiment of an enciphered (symbolic) network to detect and treat sleep-disordered breathing.

FIG. 14 shows an embodiment of the invention to enhance body function using an enciphered network.

FIG. 15 shows cybernetic enhancement of body function using enciphered functional network.

FIG. 16 shows an embodiment of the invention to transform motor function. The flowchart shows one embodiment to enhance motor (muscle control) function of the nervous system. This is illustrated for leg muscle function, for enhancement (e.g., in military or sports use) or for medical purposes (e.g., after a stroke).

FIG. 17 shows an embodiment of the invention to enhance sensory function. The flowchart indicates embodiment for enhancing sensory perception/sensation of the nervous system. This is illustrated for alertness, for enhancement (e.g., military or sports use), for medical purposes (e.g., monitoring drowsiness or coma) or for consumer safety (e.g., identifying drowsiness while driving to control a feedback device).

FIG. 18 shows an embodiment of the invention to transform sensory function. The flowchart indicates an embodiment for transposing, or enhancing sensory perception. This is illustrated for hearing, with the invention enhancing hearing and transposing hearing function to another nervous function.

FIG. 19 shows an embodiment of the invention to create a novel “cybernetic” sensory function. The flowchart indicates an embodiment for providing a sensory function that the individual does not currently possess. This is illustrated for integrating sensation from a biosensor for a biotoxin.

FIG. 20 shows an embodiment of the invention to create a novel “cybernetic” sensory function. The flowchart indicates an embodiment for using the biological nervous system for recognition of a desired pattern.

FIG. 21 shows computer hardware for machine learning.

### DETAILED DESCRIPTION

A system and method for detecting, modifying and enhancing complex functions of the body are disclosed herein. In the following description, for the purposes of explanation, numerous specific details are set forth in order to provide a thorough understanding of example embodiments. It will be evident, however, to one skilled in the art, that an example embodiment may be practiced without all of the disclosed specific details.

The invention modulates and enhances simple, complex and higher bodily functions represented in computerized fashion as a series of functional domains. In one embodiment, the function manages bodily tasks that are sensed and modulated entirely by non-medical grade devices, i.e. consumer type devices. In another embodiment, the function includes components of brain or nervous activity. A central innovation is the creation of a computerized network to represent the complex function, tailored uniquely to each individual over time. Such a representation may be called an enciphered functional network, and comprises a series of functional domains that describe normal and abnormal bodily task for that individual over time. Variations in sensed signals from the individual-normal state are interpreted by the enciphered network, as deviations, and used to guide effectors. In one preferred embodiment, the invention is applied to detect, monitor and treat sleep apnea. Other embodiments can be used to monitor and treat heart failure, manage fluid balance, manage weight to avoid obesity, or modulate alertness, mood, memory, mental performance or cognition.

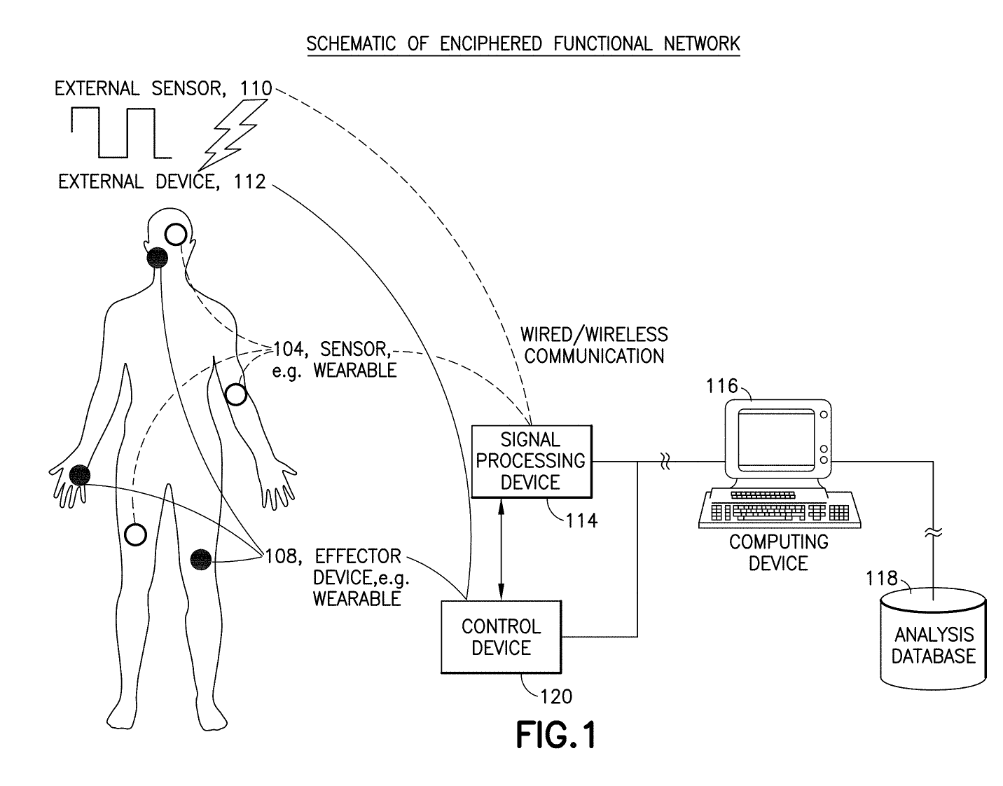

FIG. 1 illustrates an example system to modify and enhance complex body functions in a human being. Specifically, the example system 100 is configured to access external signals from biological sensors 104 and from external sensors 110.

Sensors 104 can sense biological signals, from an individual, from another individual, or from a database of signals 118. The sensors 104 can be wearable on or near the body surface, reside inside the body via an orifice such as the mouth or ear, or implanted in the body.

External sensors 110 can sense biological signals, from an individual, from another individual or from a database of signals 118. Sensed signals may arise from many organ systems including the central nervous system, peripheral nervous system, cardiovascular system, pulmonary system, gastrointestinal system, genitourinary system, skin or other systems.

External sensors 110 can provide many types of signals reflecting, but not limited to, traditional physical senses including pressure/physical movement (tactile, touch sensation), temperature (thermal information, infrared sensing), chemical (galvanic skin resistance, impedance, detection of specific ions from the skin, tongue or other mucous membranes i.e. odor, taste sensation), sound (auditory sensation), electromagnetic radiation in the visible spectrum (visual sensation), movement or vibration (a measure of muscle function and balance).

External sensors 110 can also provide information on signals just outside normally sensed ranges including, but not limited to, the invisible electromagnetic spectrum (such as near-infrared light), sound waves outside the normal physiological range for humans (roughly 20 Hz to 20 kHz) but including the range sensed by animals (for instance, dogs can sense higher frequencies), chemical stimuli, drugs or toxins. In this embodiment, the invention can extend normal functioning, for instance hearing to or beyond the audible range of individuals with the greatest acuity for hearing, or restore lost function, for instance, hearing to this range in individuals with some degree of hearing loss.

External sensors 110 can provide information on signals outside of normal sensed modalities including, but not limited to, toxins such as carbon monoxide (which is a public health risk but currently non-sensed) or excessive carbon dioxide, forms of radiation (such as alpha and beta radiation, gamma radiation, X-rays, radiowaves), biotoxins such as toxins of Escherichia coli bacteria associated with food poisoning (e.g. type 0157:H7), anthrax or other agents. This embodiment of the invention would be of value for infectious disease, military and security applications.

In FIG. 1, signals are delivered either wirelessly or via connected communication to a signal processing device 114 functioning with a computing device 116 that can access an analysis database 118. The computing device 116 and signal processing device 114 communicate with a control device 120, which in turn controls a device 108 or an external device 112. The device 108 is an effector device, which can be biological or artificial. The device 108 can be wearable by the individual or in close proximity to the individual, reside inside the body via an orifice such as the mouth or ear, or implanted in the body. The computing, signal processing and control devices with sensors and effectors together form an “enciphered functional network” (EFN).

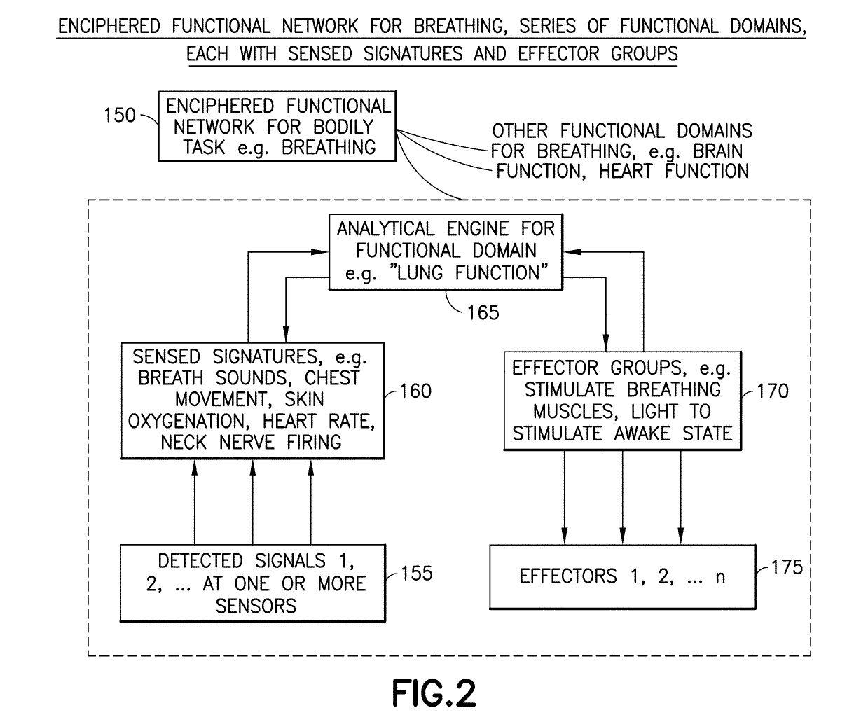

FIG. 2 summarizes the enciphered functional network (EFN) for a bodily task. The EFN may encompass one or more functional domains, each of which comprises sensors, sensed signatures for the functional domain, the analysis engine of the EFN and effector group(s) for the functional domain. At item 150 one can see the entire EFN for a particular bodily task, here illustrated for a preferred embodiment of breathing, and the functional domain termed “lung function”. Other functional domains for breathing include heart function, brain function (control of breathing centers), endocrine centers related to diurnal cycling to mention but a few. At 155 are illustrated sensors 1, 2, . . . n that are used to detect signals which together form sensed signatures 160 for this functional domain. As illustrated and discussed below, signals for lung function are diverse and include breathing sounds from a consumer or other external device, movement of the chest, movement of accessory muscles of breathing in the neck, nerve activity for these muscles (e.g. phrenic nerve, nerves in neck), airflow near the nose or mouth, oxygenation measured on the skin by optical reflectance or other means, electrical signals from the brain related to breathing or other signals.

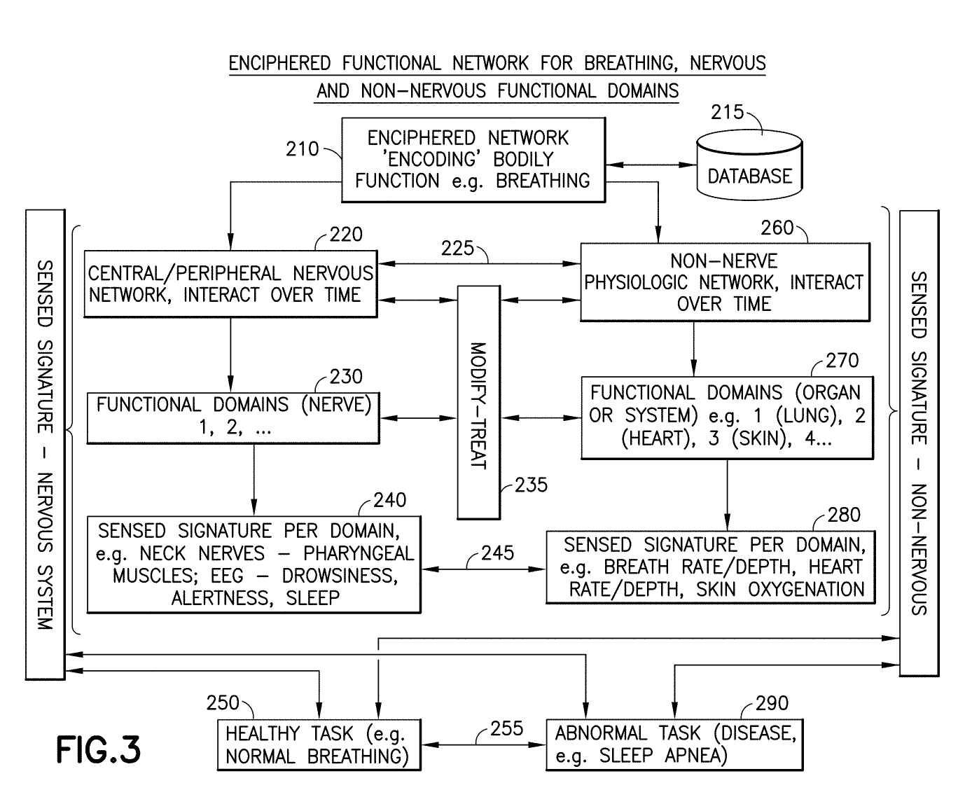

An analysis engine 165 analyzes these sensed signatures over time to form a tailored representation of this functional domain (lung function) for an individual. Many forms of analysis can be performed as discussed below. Once the EFN has tailored this representation of lung function for the individual, signals outside of the learned ranged can be detected. For instance, in one individual reduced chest movement may indicate reduced breathing while simultaneously increased neck movement may indicate use of accessory muscles of breathing and a high probability of obstructive sleep apnea. A key feature of the invention is tailored representation, because another individual may exhibit neck movement during normal sleep which does not indicate accessory muscles of breathing, and reduced breath rate during normal sleep. Of note, the enciphered network can recruit additional sensors or stored patterns from that individual or similar individuals (such as from a database, e.g. item 118 in FIG. 1 or item 215 in FIG. 3) depending on the learned or programmed behavior of the EFN.

In item 170, the enciphered functional network includes communication with an effector group for that bodily function, which in turn signals effectors 1, 2, . . . n at step 175. In this example, effector elements may include stimulation of muscles of breathing, application of light or sound (alarm, noise) to alter sleep/wake cycling. Another key element of the invention is interconnectivity and links between each element within/with the enciphered functional network, indicated by double arrows.

FIG. 3 gives more detail on the enciphered functional network for normal or abnormal functioning of a bodily task. The list of bodily tasks addressed by this invention are broad, and each typically spans multiple physiological systems (functional domains). Bodily tasks may include but are not limited to sleep, sleep disordered breathing, cognition, mental performance, response to obesity, response to heart failure.

In FIG. 3, a preferred embodiment indicates EFN for the bodily task of breathing 210, comprising nervous system 220 and non-nervous system (non-neural) 260 networks. The networks 220, 260 comprise respective functional domains 230, 270, each defined by sensed signatures 240, 280 based on a variety of sensors. This produces nerve and non-nerve signatures for the body function, which can be normal 250 and abnormal 290 —or desired 250 and undesired 290. It should be noted that the networks can interact via interactions 225 and signatures may be inter-related by expected (i.e. from physiology) or learned computational relationships 245.

The analysis engine of the enciphered functional network uses various methods including implementations of artificial intelligence (machine learning, perceptron, deep learning, autobot, and/or fuzzy logic circuits), comparison against previously stored patterns, classification schemes, expected algorithmic relationships or heuristic approaches. Rule-based systems include a database of solutions for sensed signatures, such as dermatomal distribution for shoulder nerves, fluctuations in skin reflectance indicating oxygenation, variations in auditory sound intensity that separates breathing from snoring, and normal ranges of heart rate and others familiar to one skilled in the art.

In one preferred embodiment, machine learning is accomplished via neural networks (e.g., 3 layer back-propagation networks, multi-level networks or other designs) and techniques of deep learning. Numerically, networks are defined:

(i) By node interconnects, which vary between layers of nodes (artificial neurons). Nodes are typically represented as networks, and there may be many layers and many variations in the number of nodes in input, hidden (internal) and output layers. Nodes can be connected to all nodes in layers above and below, but differential connections can also be implemented;

(ii) How nodes are connected, i.e. weights of their interconnections, which are updated in the process of learning;

(iii) A mathematical activation function, summarizing how a nodal interconnection weights input to output. Typically, the activation function of each node ƒ(x) is a composite of other functions g(x), which can in turn be expressed as a composite of other functions. A non-linear weighted sum may be used, i.e. ƒ(x)=K (Σᵢ wᵢ gᵢ (x), where K (the activation function) may be sigmoidal, hyperbolic or other function.

Various connection patterns, weighting, node activation function and updating schemes can be selected, and specific forms are optimal for different enciphered networks. The enciphered network linking EEG, cardiac and respiratory signatures to alertness, or linking weight, skin impedance, respiratory rate and cardiac output to heart failure status, for example, is more complex than a network linking recorded sound analysis with sleep disordered breathing. Recent approaches to complex tasks use recurrent neural networks, in which connections between nodes form a directed cycle to enable dynamic temporal behavior and enable complex tasks such as modeling alertness.

Alternative forms of adaptation of the enciphered network may use algorithms in the “if-then-else” formulation to link sensed signatures with defined behaviors. Several other forms of machine learning can be applied, and will be apparent to an individual skilled in the art.

An important feature of such approaches is that they do not need a priori knowledge of the specifics of human pathophysiology, but instead associate (‘learn’) patterns of sensed signatures in health (normal functioning) and deviations from these patterns in disease (abnormal functioning). They are thus well suited to complex bodily tasks that are often defined incompletely by detailed pathophysiological studies, yet still need to be monitored and treated.

The enciphered functional network can provide a computerized implementation of bedside examination by a physician—it objectively represents “good health” or “looking good”, i.e. normal skin color and blood perfusion for an individual, normal breathing for an individual, normal muscular movement for an individual and other intangible physical signs. The analysis engine of the enciphered functional network then addresses the tractable problem of identifying when sensed signals deviate from any baseline state for that individual.

The novelty of using the enciphered functional network and sensed signatures to monitor health is illustrated by the following analogy. A “high tech” approach to identifying health in an advanced hospital may find that an individual has a cardiac output of 5 l/min, normal polysomonogram with normal EEG and other parameters, normal arterial oxygen and carbon dioxide concentrations, normal cardiac nuclear stress test, and hemoglobin and other blood parameters within normal limits. A comprehensive embodiment of the current invention may come to the same conclusion through normal values of the following domains for that individual: heart (normal heart rate, normal variations with no abnormal drops in oxygen saturation during activity); lung (normal breath sounds, no wheeze, no noisy breath sounds while awake, no loud snores or apneas at night, normal oxygen saturation); general health (normal scleral color, normal diurnal temperature fluctuations, steady weight, good activity profile and normal diurnal heart rate/oxygen fluctuations). Thus, this individual appears “in good health” on bedside examination by a physician and also by this invention, which could reside on a consumer device for easy access. Thus, this invention is designed as a screening tool and ‘personal health assistant’. It is not designed to replace advanced and invasive medical examination and testing if indicated, but the device can alert the user to abnormal parameters which may accelerate referral to medical providers if needed. This could be a telehealth provider, as well as traditional provider networks. The invention thus may have value in medically underserved regions, e.g. in rural areas in the U.S. or in countries with less ready access to advanced medical care. The invention may also improve medical care by providing objective, repeatable assessment of many parameters of health tailored to that individual.

One important distinction from the prior art is that individual tailoring enables this invention to identify sensed signatures that may be normal for one individual yet abnormal for another. This invention thus advances “personalized medicine”, or “precision medicine” which are often defined at the genetic level but are often undefined for the whole individual. This invention enables robust implementation of precision health at the clinical level, based on how a function affects measureable organ systems for that individual. This clinical science is novel.

Using another analogy, the symbolic model of simple and complex tasks by the enciphered functional network may at times be akin to representing visualization by an “impressionist” painter rather than a detailed physiological representation—by one trained in the “realist” school. Again, this approach is based on the premise that in addition to the primary physiological systems required for a task, it is difficult to precisely define, secondary networked regions that become involved.

Associations of sensed signatures with normal function 250 in a patient specific range enables the invention to detect abnormal function 290 as signatures outside this range. The enciphered functional network is optimized when learning algorithms repeatedly classify interactions 255 between sensed signatures for normal 250 and abnormal 290 functions. This interconnectivity is optimal, and its complexity makes the system ideally suited for computational machine learning paradigms to modify and treat the networks 235.

In FIG. 3, a database 215 of learned representations for the individual over time, or for multiple individuals may enhance personalized diagnosis and therapy. This can be used to enhance diagnosis and therapy via the EFN for that individual.

The database 215 of learned networks (representations) between individuals is another core resource of the invention—a digital network of different sensed modalities for a function in defined populations that may be used to monitor and treat disease or improve performance. For health care or screening purposes, database 215 can be encrypted as well as de-identified, but if individual consent is obtained, e.g., in military or Institutional settings, abnormalities can be traced from or applied to specific individuals to improve their performance in the population. This forms the basis for a novel approach to crowd-sourced health or wellness screening, crowd-sourced disease monitoring, and crowd-sourced delivery of therapy.

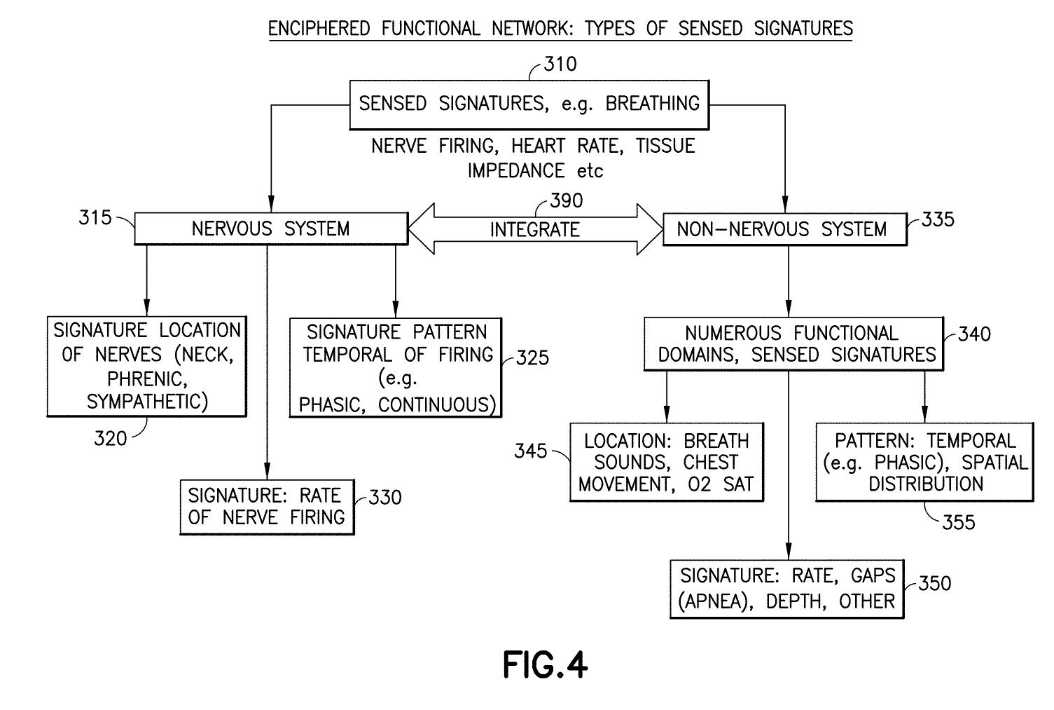

FIG. 4 provides detail of signatures sensed 310 by the invention to represent a given bodily task tailored to an individual. The task described here for the preferred embodiment of breathing. Functional domains for the body task are broadly classified as nervous system related 315 and non nervous system related 335, which may be integrated 390. Sensed nerve signatures 315 typically represent the sensing location 320 (for instance, nerves in the neck for accessory muscles of breathing, the phrenic nerve for diaphragm activity, or sympathetic nerve firing which may indicate a stress response during sleep apnea), patterns of activity 325 (e.g., periodic with a certain frequency spectrum, or more complex and potentially represented non-linearly by fractal dimension or measures of entropy), or rate of firing 330 (e.g., the fundamental or “dominant” frequency of a spectrum or first peak on an autocorrelation function).

Numerous other nerve-related parameters are possible, e.g., nuclear scans of neuro-tissue function, e.g., MIBG scanning for autonomic ganglia, metabolic quantification using positron emission tomography based sensor information, serum levels of norepinephrine and other nerve-related signatures familiar to one skilled in the art.

Nervous and non-nervous functional domains are optimally integrated 390 for any complex bodily function, yet the distinction may be useful as embodiments utilizing nervous functional domains 315 may be implemented by electronic sensors and electronic effector devices, and form a biological neural network which can be mimicked by an artificial neural network in the enciphered functional network.

Non-nerve functional domains 335 may be multiple 340 and typically have one or more defined signatures, e.g., hypervolemia is detectable by reduced electrical impedance of tissue, sympathetic activation via “clammy skin”—reduced galvanic skin resistance and altered ionic composition, apnea via reduced oxygenation measured as reduced skin absorption in the near-infrared end of the electromagnetic spectrum. These signatures can also be characterized by spatial location 345, rate 350 and temporal patterns 355. Locations 345 for breathing include non-contact sensors of breath sounds (e.g. smartphone), movement sensors on the chest or neck to measure breathing, oxygenation on the skin. Signatures 350 for breathing include absence of breath sounds (apnea), loud breath sounds (snoring, arousal), irregular breathing movements (e.g. Cheynes-Stokes breathing). Patterns of these signatures include rapid, slow and other patterns. Numerous other parameters can be measured currently and others may develop in time and be naturally incorporated into this invention by an individual skilled in the art, e.g., tissue concentrations of neurohormones such as B-type natriuretic peptide, cortisol or prolactin from a pharmacological sensor, signal intensity from a photodetector to detect drug concentrations in skin or cutaneous blood vessels, drug or alcohol levels in exhaled breath from an oropharyngeal sensor, drug or alcohol levels in urine from a urethral sensor, cell counts in a tissue sample e.g. sperm counts to test for infertility, and other sensors relevant to the functional domain under consideration.

Sensed signatures illustrated in FIG. 4 represent the functional domains of that bodily task for an individual person. This forms a type of digital or computerized phenotype for that bodily function. It is recognized that nervous and non-nervous physiological elements can be deeply integrated biologically, but this formulation is a convenient approach to parameterize complex physiology into tracks that can be measured, mathematically modeled and learned. Other more integrated formulations are possible.

It is important to note that neither all illustrated nor possible signatures are required for the invention to work, i.e. the minimum embodiment. For instance, heart failure can be monitored from the simple measure of weight gain alone. Sleep apnea can be detected from one primary signal—prolonged periods of time without breathing (other signals being supportive). This invention uses the enciphered functional network to weight the most important signature(s) for that individual, either explicitly or implicitly (e.g. via learning), and use whatever signatures are currently available.

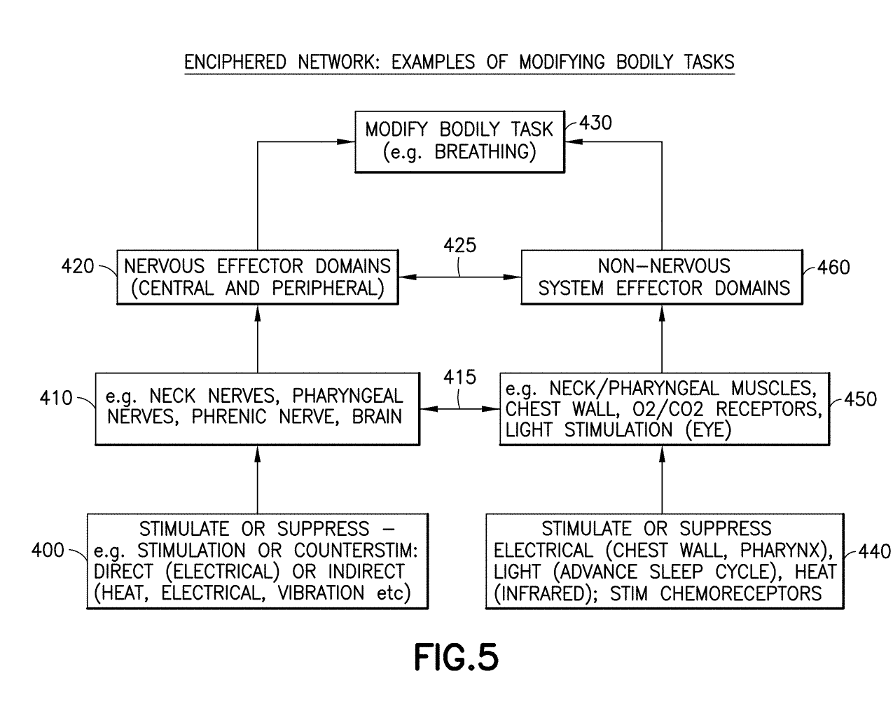

FIG. 5 illustrates modification of the bodily task by effector functions, tailored to sensed signatures for that task. Modifications may comprise therapy, e.g. for sleep-disordered breathing, but may also comprise enhanced normal function, e.g. in sleep quality or alertness. Modification through the enciphered network operates using a feedback loop, in which effector responses are measured by subsequent changes in sensed signatures, to prevent excessive modification. Nerve-related domains 420 can be modified by direct energy delivery 400 to stimulate or suppress a domain. For instance, competitive—stimulation (‘counter’ stimulation) of skin on the abdominal wall (e.g., vibration via a piezoelectric device, heat via an infrared generator) may suppress the sensation of pain in organs innervated by visceral nerves of lumbosacral origin (lower back). Domains 410 may thus lie in the peripheral nerves, such as neck nerves to relieve obstructive sleep apnea or the phrenic nerve to stimulate breathing in central sleep apnea, or central nervous system such as scalp stimulation to modify cranial nerves or light delivery to modulate the ophthalmic nerve or (indirectly) pineal gland activity. In this way, the bodily function can be treated, enhanced or otherwise altered 430. Non-nerve domains 460 can be modified in many ways 440 including vibratory stimulation via a piezoelectric device to stimulate a muscle, infrared heat to reduce muscle spasm to modulate various domains 450 and 460 to modify the bodily function 430. Again, the response to modification from effector functions is individually tailored and monitored by sensed signatures for that bodily task to ensure that excessive and/or deleterious effector functions are not delivered.

Modulation of nerve-related domains 410 can be linked to modulation of non-nervous domains by modulation connection 415. Moreover, the central and peripheral nervous domains 420 are typically linked to non-nervous system domains 460 by connections 425 which may form other functional domains (e.g. function of adrenocortical glands links the sympathetic nervous system with the endocrine effects of cortisol secretion which impact weight, glucose control, mood, alertness and sleep).

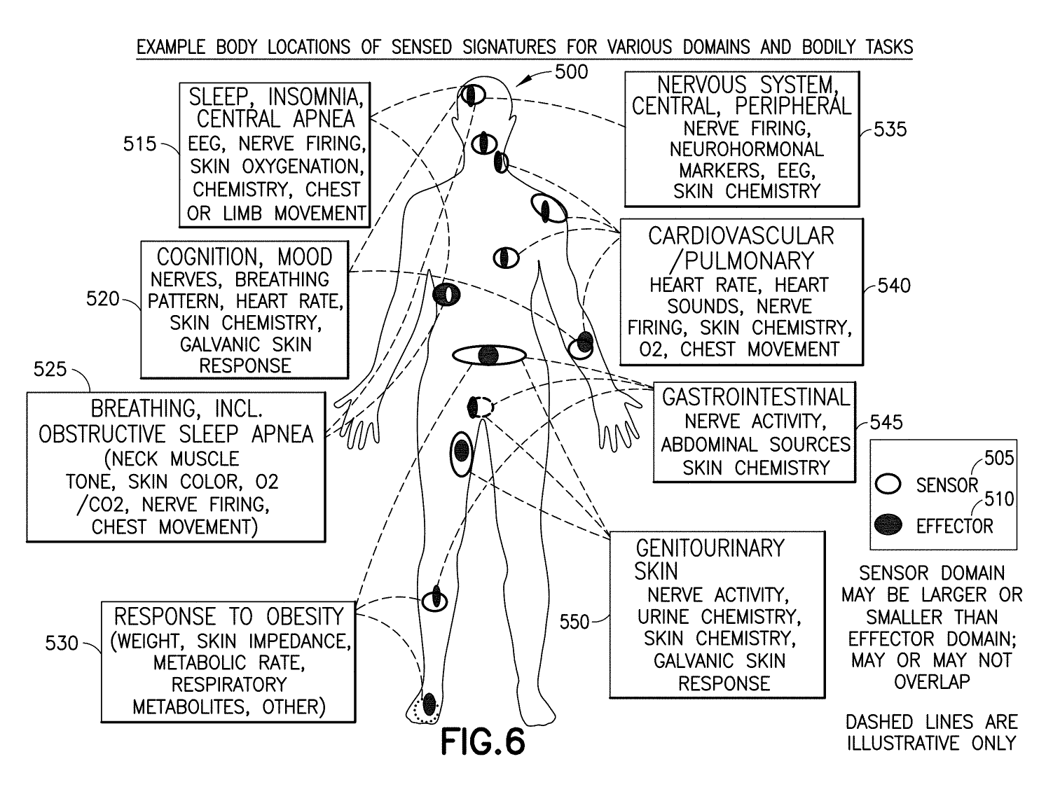

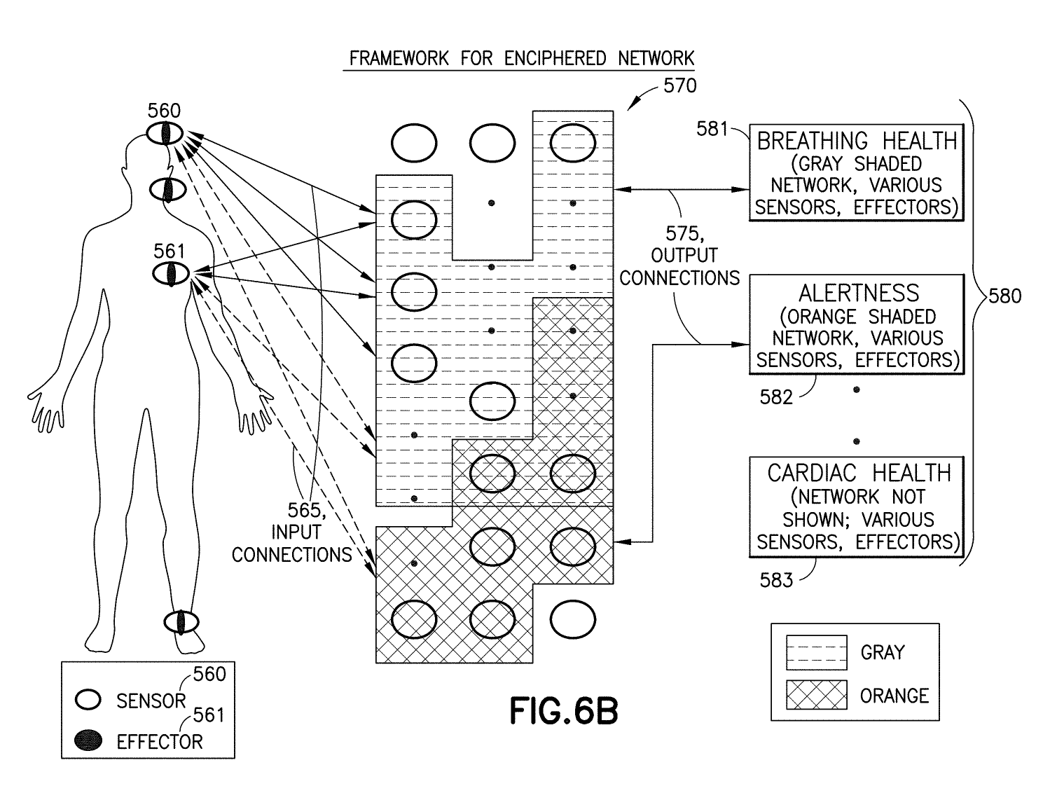

FIG. 6 indicates several potential body locations 500 for sensors and effectors. Bodily functions can be measured by sensor sites 505 and/or modified by effector sites 510. Sensor sites are shown by open (white) regions, and effector (modifying) sites by filled (black) regions. Their relative physical sizes vary in each individual and are not shown to scale. FIG. 6 indicates sensor locations on the body 500 to detect signatures of the nervous 535, cardiovascular 540, pulmonary 540, gastrointestinal 545, genitourinary 550, skin 550 and other organ systems. Body tasks measured and/or modified by the enciphered functional network include, but are not limited to, sleep and central sleep apnea 515, cognitive performance 520 such as alertness, obstructive sleep apnea 525, and the bodily response to obesity 530. The variety of sensors, sensed signatures, functional domains and bodily tasks are indicated by way of example and not to limit the scope of the invention. These are discussed in more detail with regards to other figures in this disclosure.

FIG. 6B illustrates a preferred framework for the enciphered functional network. The main elements are 560 arrays of sensors, 561 arrays of effectors, 565 input connections to 570 a processing network. 575 shows output connections from the processing network to health application layers 580 for various bodily or health tasks, including breathing health 581, alertness 582 and cardiac health 583.

The processing network 570 links the sensor or effector arrays with health states using different implementations of logic. If this is machine learning, then in training the health state feeds backward into the network (hidden layers) to alter weights and associations. For breathing health 581, the sensor array 560 provides sensed signatures (e.g. normal breathing, normal oxygenation, normal heart rate variability) that are linked repeatedly with normal breathing over time for that individual. Sensed signatures from the sensor which deviate from this pattern are now classified as abnormal breathing. The same is true for other body tasks/health states, e.g., alertness, cardiac health.

The processing network 570 may be rule-based, in which case sensed signatures (sensor states) outside of normal values are flagged as ‘abnormal’. Normal values can be programmed (rules) or learned (hybrid, adaptive-rules). The processing network 570 may also be heuristic-based, database lookup or based upon other associations.

Processing networks 570 may overlap for various body tasks or functions, as depicted by the overlap in shaded boxes. For instance, a rapid heart rate may be abnormal for breathing health or for cardiac health. On the other hand, the other sensed signatures provide context, because a rapid heart rate may be normal for exercise or alert states.

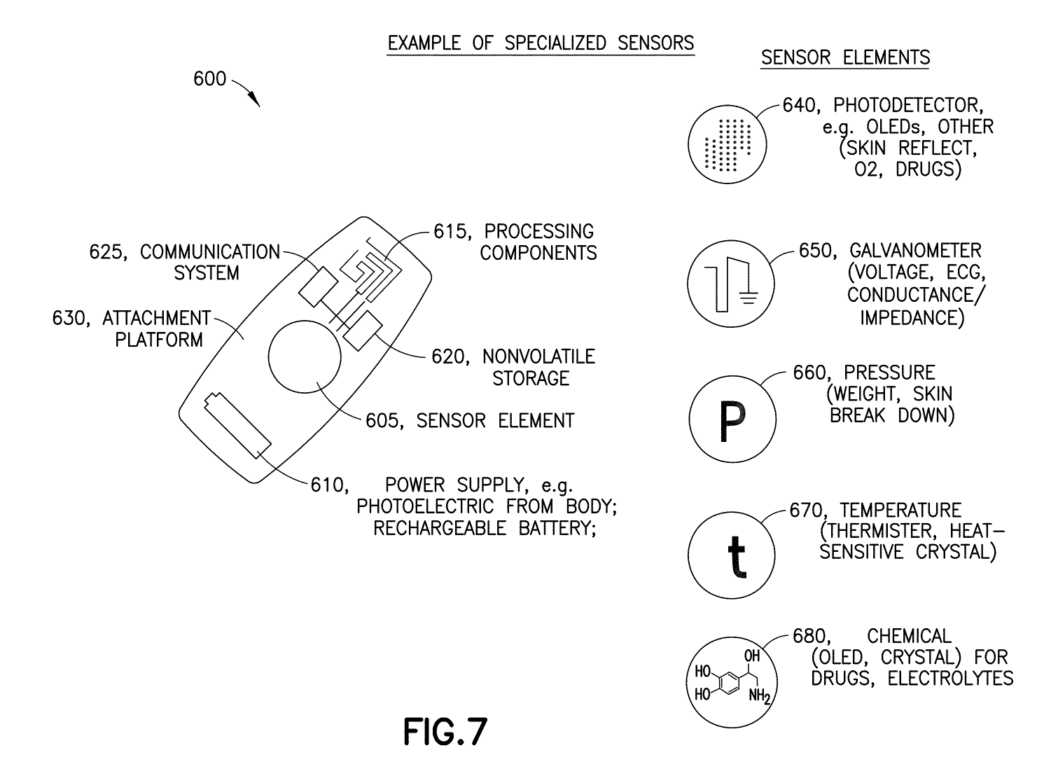

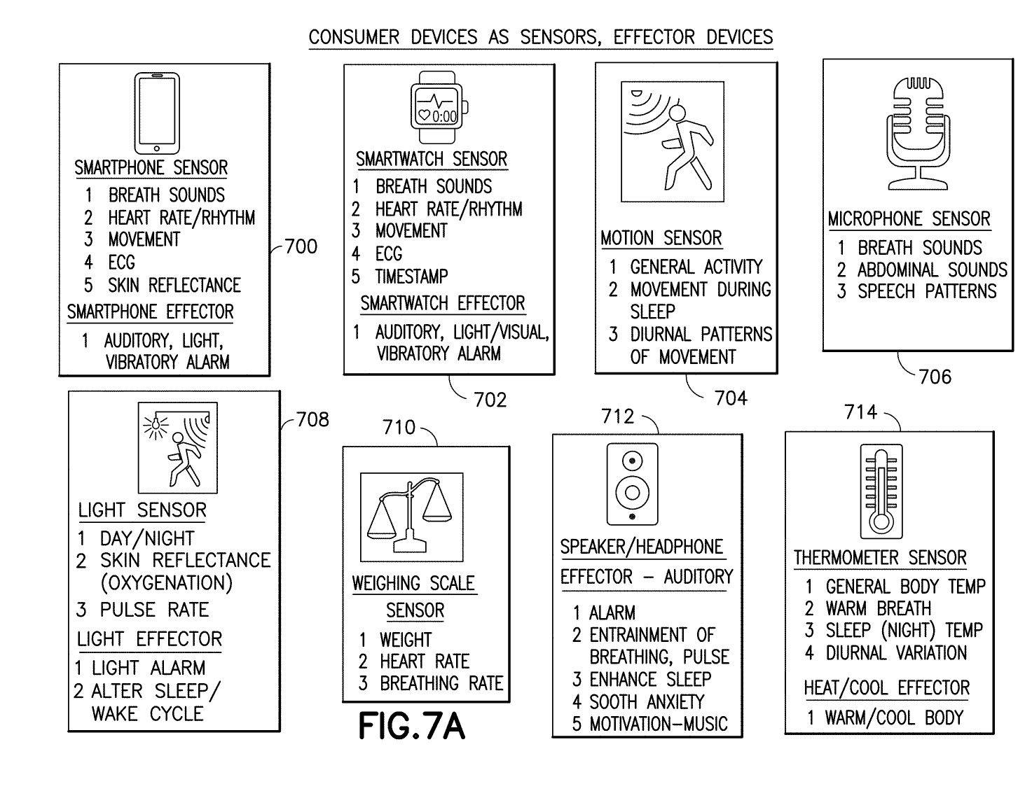

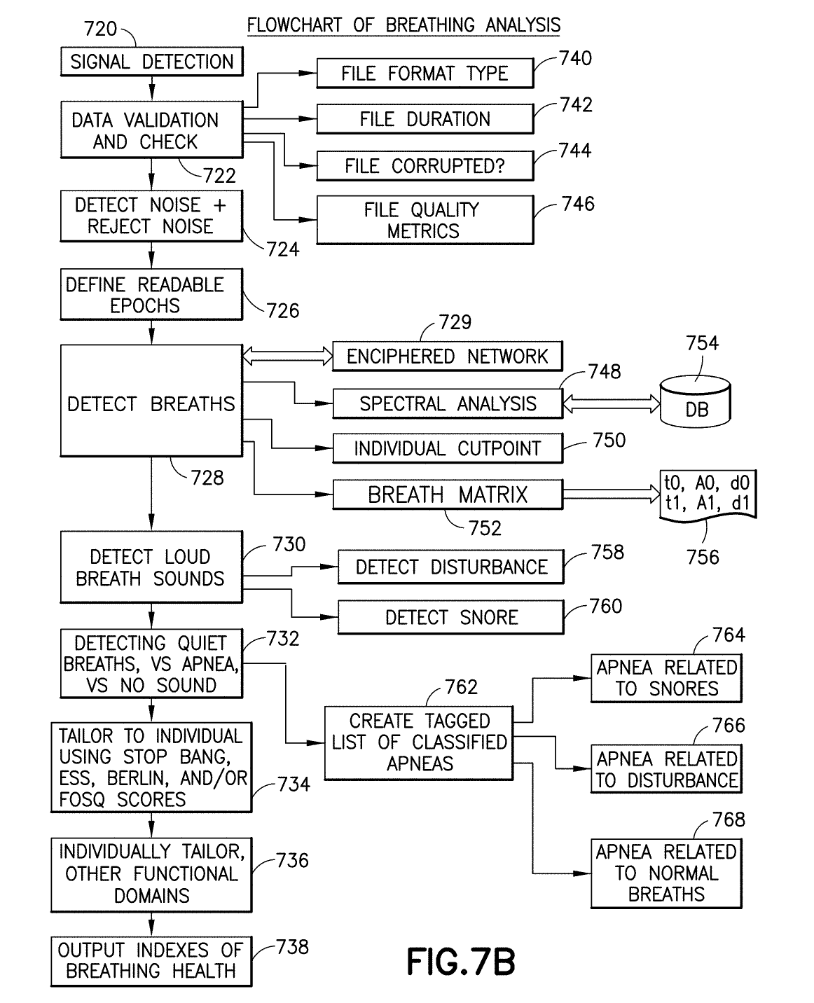

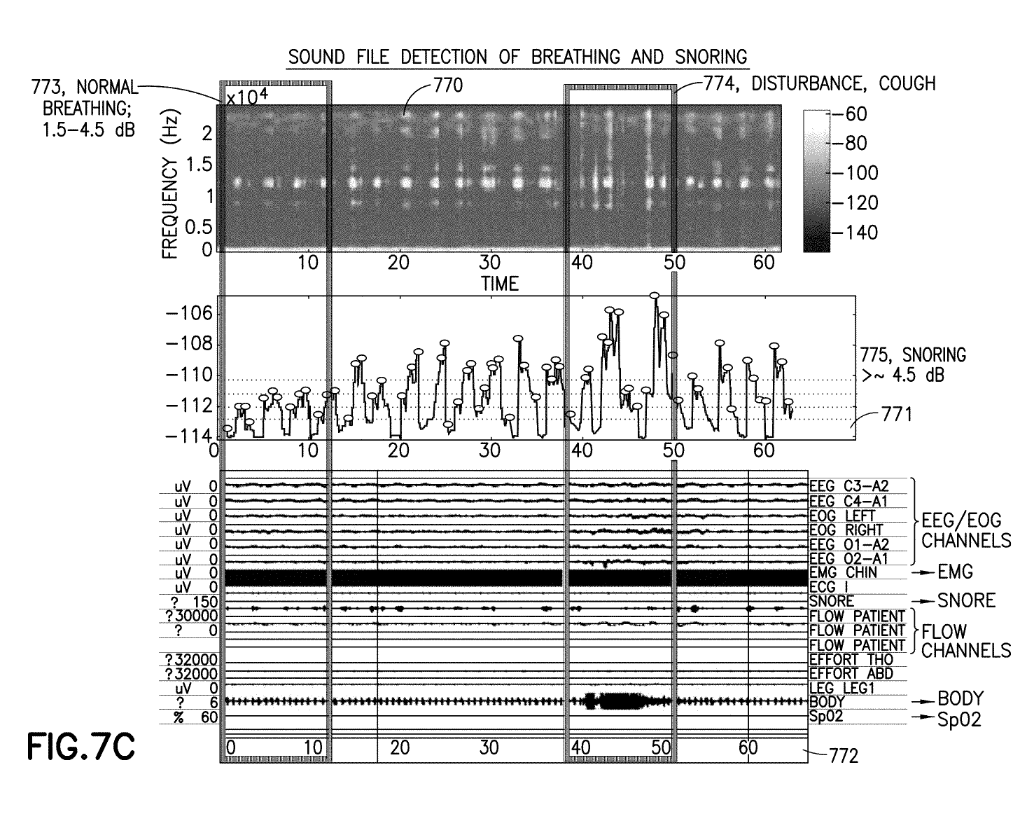

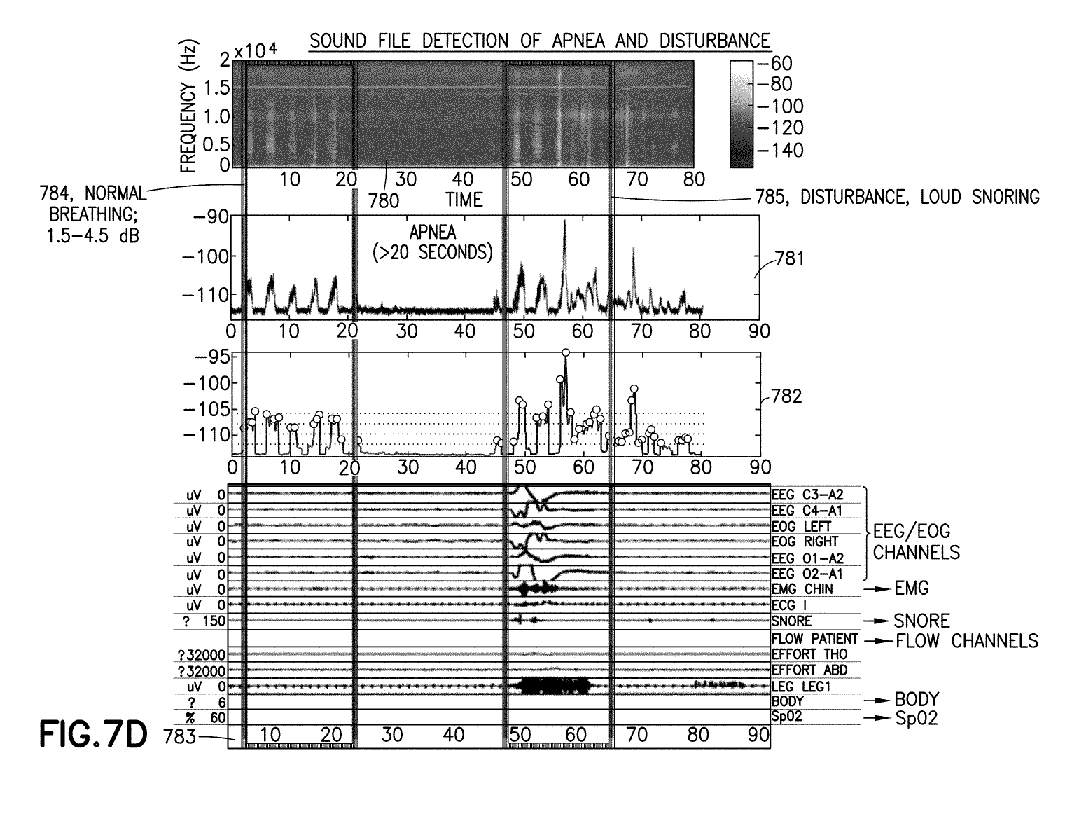

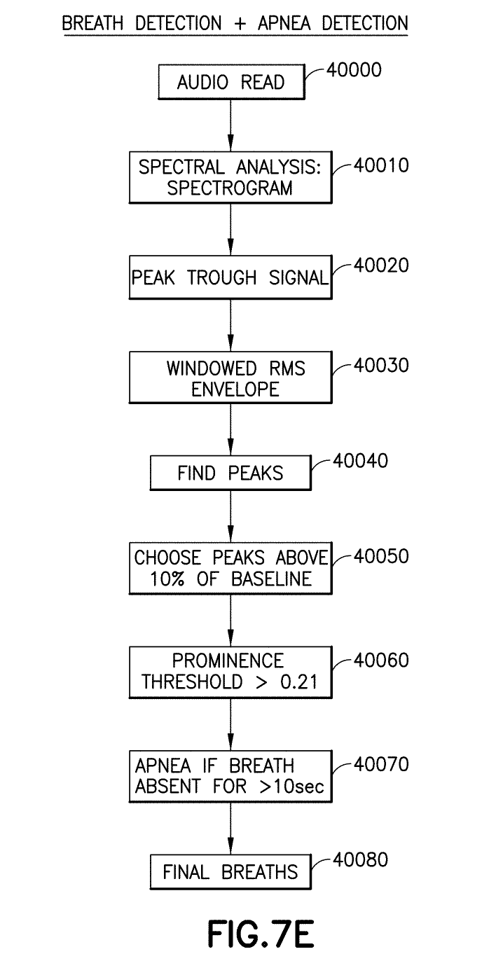

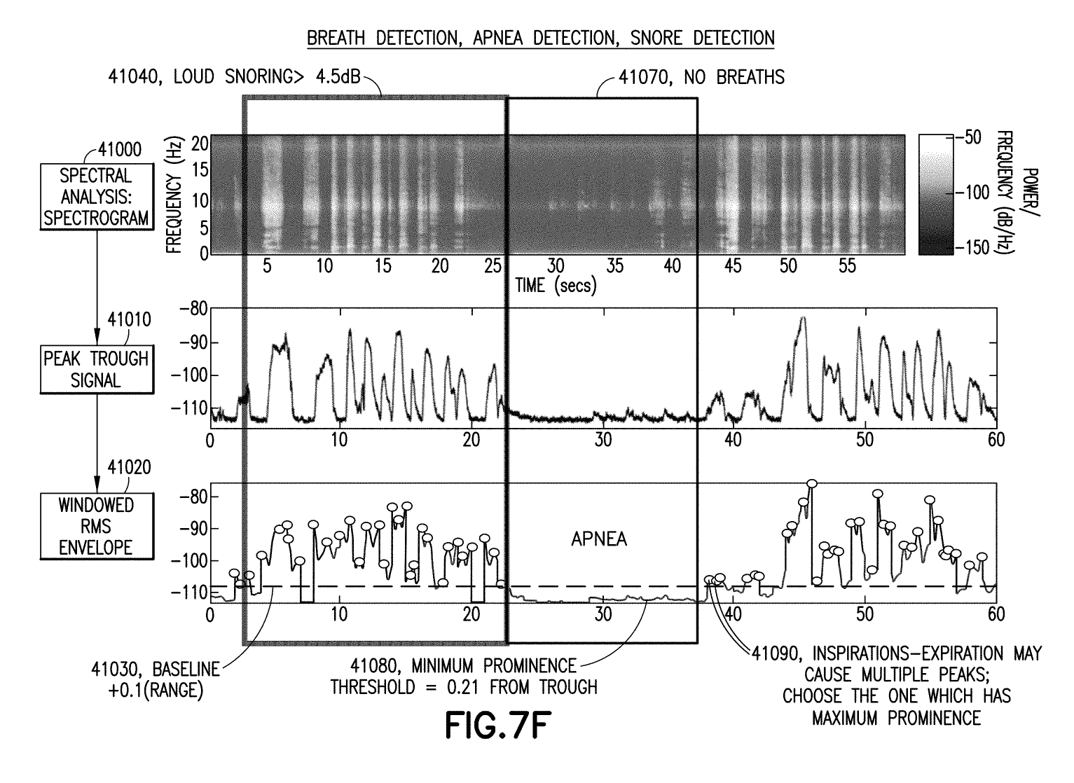

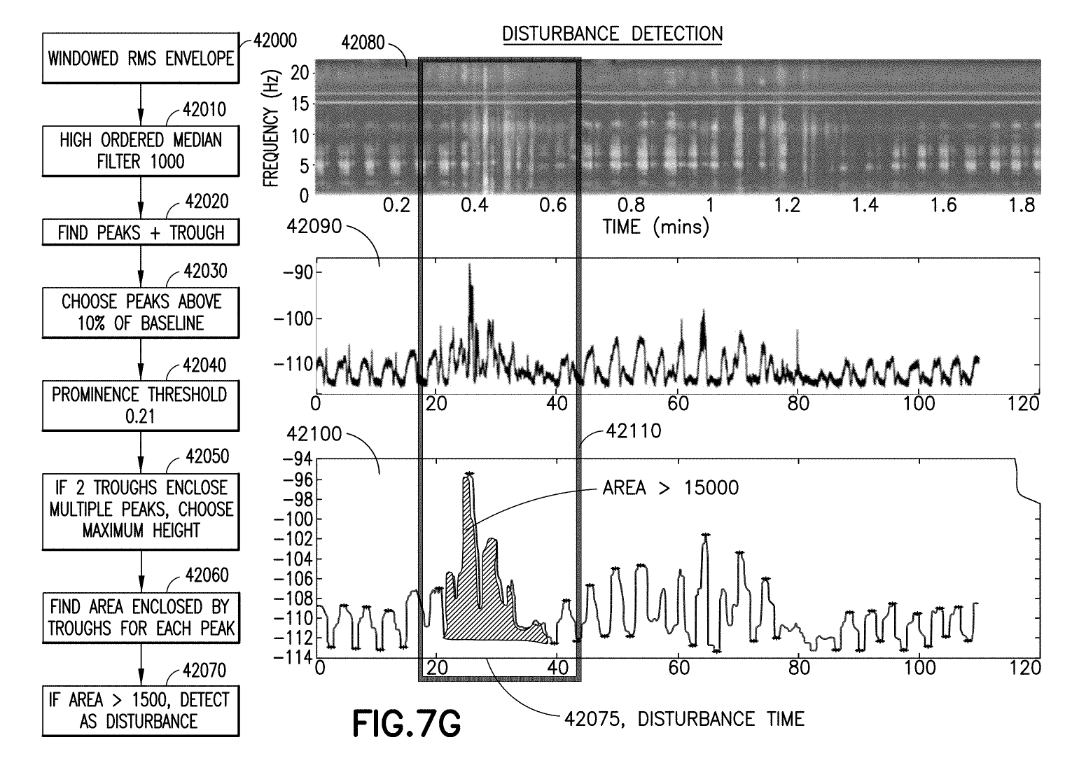

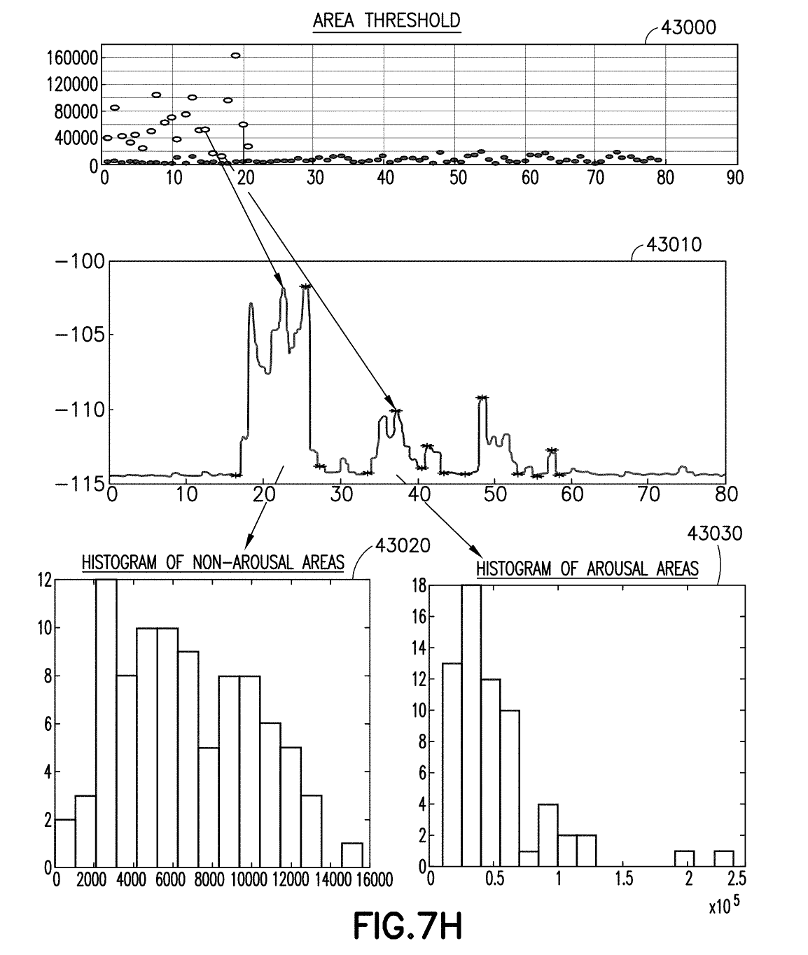

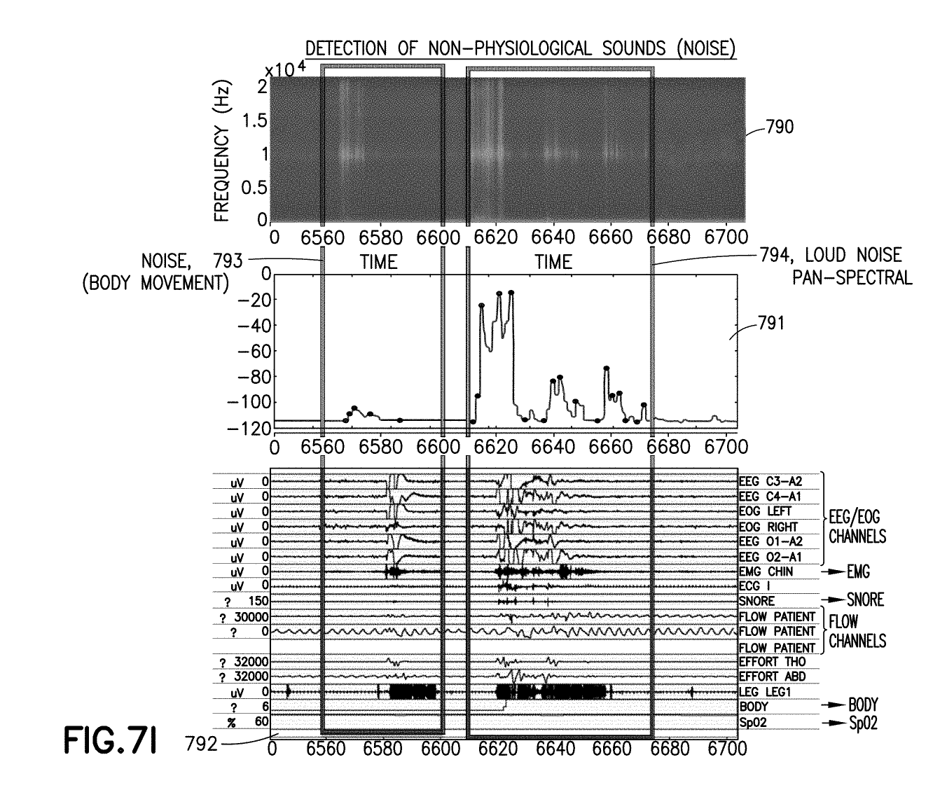

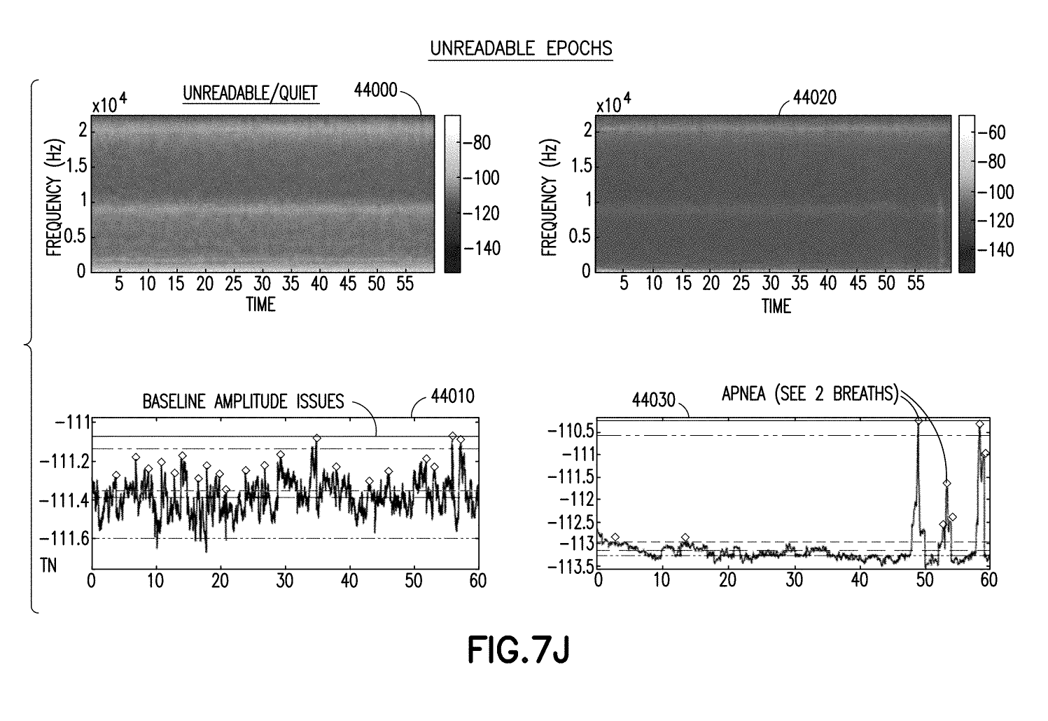

FIG. 7 illustrates an example of a body sensor 600, comprising sensor element 605, power source 610, processing components 615, nonvolatile storage 620 (e.g., E2PROM), communication element 625 on a structural platform 630. Several types of sensor elements are illustrated. Sensors include, but are not limited to, photosensitive sensors 640 to detect skin reflectance (indicating oxygenated hemoglobin, perfusion including pulse rates), galvanometers 650 to detect skin impedance or conductance (a measure of body chemistry), transcutaneous or invasive nerve activity (neural electrical activity) or muscle electrical activity (myopotentials), pressure detectors 660 (to detect pressure, e.g., weight, mechanical joint movement or position), thermal detectors 670 to detect temperature (a measure of metabolic activity and other disease states), and chemical detectors 680 to perform assays for norepinephrine or drugs, body pH from the skin, mouth, or elsewhere in the gastro-intestinal or genitourinary tracts, enzymatic profile in the gastrointestinal tract, DNA profile (for instance, a gene chip on the lining of the mouth), and other sensors such as for heart rate, ventilation (breathing).

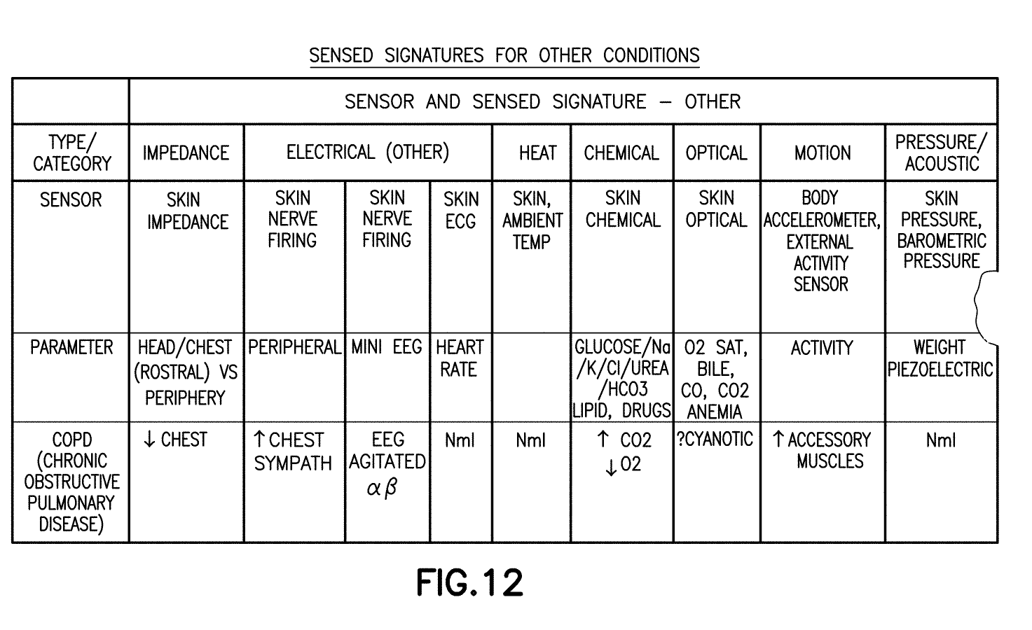

The invention can also use external sensors (FIGS. 1, 12-18) that provide a variety of extrinsic or artificial signatures (FIGS. 12-18) to provide cybernetic sensor inputs or effectors to the enciphered functional network.

FIG. 7A indicates several consumer devices that detect signals and can provide sensed signatures for important functional domains. Consumer devices include, but are not limited to, smartphones 700, smart watches 702, clothing-related sensors, home motion sensors 704, microphones 706, light detectors 708, weighing scales 710, dedicated sound generators such as loudspeakers or headphones 712, thermometer 714 or others. Such devices detect a broad array of sensed signals, if subjected to appropriate processing and transformation by the enciphered functional network.

In a preferred embodiment, recorded sounds from a smartphone 700 in FIG. 7A are used to detect normal breath sounds, lack of breaths (apnea) and abnormal breath sounds including obstructive sounds and snoring. To accomplish this from a consumer smartphone with no medical devices, the invention and enciphered functional network reduces noise and filters raw sound files, applies physiologically-derived algorithms to detect breaths relative to noise, speech, other physiological sounds. The algorithms also separate sounds from a separate individual (e.g. bed partner), determines their relationship to normal patterns for that individual, and can hence detect disordered breathing. Similar functionality can be achieved with a smartwatch 702, or devices such as a consumer microphone 706. In an alternative embodiment, consumer motion sensors 704 can indicate movement, from which the invention can determine the presence or absence of breaths as above. In a related embodiment, a motion sensor 704 on a bed, chair or other support can detect movement which the invention can identify as breaths. In yet another embodiment, a thermometer 714 can identify fluctuations in temperature near the mouth or nose, which the invention can use to detect breathing and lack of breathing as above. In yet another embodiment, a light source 708 can illuminate the individual's chest at various wavelengths including far red and near infrared light (more penetrating than visible light), and reflected light can indicate chest wall or neck movement which the invention can associate with breathing to determine normal/abnormal breathing. Other embodiments from consumer devices will be apparent to others skilled in the art.

Other functional domains can be defined by sensed signatures from the array of sensors in FIG. 7A. For instance, diurnal variations in overall or regional body temperature from the thermometer 714 can be used by the invention to monitor sleep, awakeness and general health. Thermal sensors can be in body clothing, on a watch or other near-body location. Near-infrared sensors/cameras can be embedded in walls of a house or other convenient location. Motion sensors 704 can be used to determine when the individual is sleeping versus awake, and active versus sedentary. Sensors can be wearable on shoes/clothes, or fixed in a residence, bed or car, for instance. Weighing scales 710 can provide sensed signals to help in management of weight (obesity) or fluid management (heart failure). For regular assessments, weighing/pressure sensors can be part of smart car seat, smart bed, shoes, in the floor of a room of a house or in other situations. Other functional domains that can be defined by the wide array of available sensors are outlined in the specification, and will be apparent to others skilled in the art.

In several embodiments, sensed signals from sensors illustrated in FIGS. 7 and 7A will require a personal identification tag to ensure that data is being analyzed from the individual in question, results are communicated to that individual, and/or effector responses are delivered to that individual. This can be accomplished in hardware or software. Hardware embodiments include sensors of biometric information specific to that individual, such as a fingerprint, retinal scan, picture of the iris or unique facial features, composition of sweat, salivary composition (for sensors in the mouth), mucous composition (for sensors in the nostrils or elsewhere in the airway), sensors to analyze heart sounds, breath sounds or speech patterns. Software embodiments include spectral analyses, pattern matching analyses or correlative analyses of these sensed biometric signals compared to known signals from that individual. Known signals from that individual can be sensed at the time of data recording, from a prior stored event, or from a database. In the preferred embodiment of the invention to monitor breathing health, sound files are analyzed after confirming that biometric data matches that from the individual in question, an index of health or disease is made available to that individual and his or her designees, and effector responses are delivered after confirming a match in biometric data to the correct individual.

Consumer devices in FIG. 7A can also be effector devices for the enciphered functional network. For instance, the smartphone 700 can provide an audible, light-based or vibratory alarm to awake an individual if sleep apnea is detected. These or external devices, e.g. a computer controlled light source, can be activated to advance or retard the sleep/wake cycle tailored for an individual with disturbances of sleep or sleep-related breathing. A smartwatch 702 can provide a vibration signal, auditory alarm or other signal to the individual as an effector response. A loudspeaker 712 can provide stimuli to alter activity, sleep and other functions. A heating or cooling element 714 can alter the propensity of the body to sleep, or alter diurnal cycling. Other applications for the health and disease states in this application will be evident to a person skilled in the art.

FIG. 7B indicates a preferred embodiment of the invention, which analyzes breathing-related files to monitor and treat the bodily task of breathing. One specific preferred embodiment uses only consumer equipment, records sound files using built-in consumer hardware of a smartphone, uses software on the phone or cloud computing to analyze sound to detect breath signals, generates breath signatures for that individual which can be used to detect and manage breathing disorders. In another preferred embodiment, consumer equipment added to the phone is used to sense signals including but not limited to chest movement, oxygenation, and/or brain activity, to generate other individual signatures. In yet another embodiment, medical grade equipment is used to record signals and generate signatures for the bodily task of breathing. In different sets of embodiments, the invention uses consumer equipment or medical grade equipment to manager other bodily tasks.

In FIG. 7B, signals are detected in step 720. This includes an individual recognition/ID process, then a calibration step at the start of each detection period. For instance, in one preferred embodiment, the sound intensity of normal breaths is captured, calibrated to distance from the smartphone to the individual, and to sound intensity in that individual at that time. Data is checked and validated in step 722. The first file tag is a check of digital file format 740, such as “.wav” for sound files. Other appropriate file types can be analyzed for breath signals including but not limited to “.mpg” movies of chest wall motion, “.mpg” movies of neck/pharyngeal obstruction, other file types encoding chest wall movement (e.g. files from piezoelectric sensors), commercial home motion sensor files, or file types encoding oxygenation status from skin reflectance or other sensor. File duration is read 742 and files less than a certain duration may be excluded. For analysis of sleep disordered breathing, a typical threshold for adequate duration is >4 hours of recording. File segments that are corrupted are flagged in 744 and file quality metrics are generated in step 746.

In a preferred embodiment, step 722 checks data for adequacy for breath analysis, such as the presence of periodic activity at the typical rate of one breath every 2-5 seconds (i.e. 0.5 to 0.2 Hz). Another check is whether the periodic activity is likely to be breathing. For sound files, this may include a typical duration of each event of 0.5 to 3 seconds (duration of a breath). For sound files, individual breaths also exhibit typical spectral characteristics, often in the range of 5-15 kHz loudest at 500 Hz-12 kHz, which separates a breath from noise and some aspects of speech. If assessing breathing from chest movement sensor files, the rate should be the same but duration of chest movement will be longer than airflow indicating breath sounds (the chest moves before air begins to flow, and may continue moving after airflow stops). Indexes of movement may be similar for the abdomen, in individuals who use “abdominal breathing” to assist the mechanical function of breathing (ventilation). Notably, indices of breathing movement will differ in periodicity, amplitude, relationship to other sensed signals (e.g. fluctuations in oxygen saturation, variations in ECG amplitude, heart rate) and other properties from non-breathing movement of arms, head or legs, for instance. Metrics can be assessed by spectral decomposition 748, autocorrelation analysis (checking the time shift or amplitude of peaks), or other pattern matching, by individual cutpoints 750, or from a matrix 752 any of which can be stored on database 754 or external medium 756. In the preferred embodiment, the enciphered functional network tailors breath analyses to an individual, and registers ‘normal’ for that individual under conditions such as times of day (longer and slower breaths at night), exertion (shorter and faster breaths), REM sleep (more irregular breath rate and depth compared to Non-REM sleep) and so on.

Step 724 detects and rejects noise in order to define unreadable epochs. For the preferred embodiment of breath analysis, noise includes sound, chest movement or other signals that do not meet typical criteria for breathing. For instance, a periodic signal at ten times per second (10 Hz) is not human breathing, and is excluded using methods in the art including spectral filtering using Fourier and Inverse Fourier transforms, wavelet analysis and other methods. Some filters are absolute (e.g. the example of breathing rate >5-10 Hz), and some are relative and individualized, e.g. breathing rate in an particular individual may never be >2 Hz during surveillance. After excluding noise, potentially valid signals are passed to the next step e.g. periodic signals at 0.8 Hz that are low amplitude, which could potentially indicate fast shallow breaths (during exertion) or noise. Other signals, e.g. movements of activity, rapid fluctuations in oxygenation or rapid heart rate, could complete the signature of exertion and allow this signal to be analyzed. Conversely, rapid high amplitude signals (from breath sensor or chest movement sensor) without concomitantly high heart rate, oxygenation fluctuations etc are unlikely to be breaths and may be rejected after analysis by the enciphered network. This analysis ends with defining readable epochs in step 726.

Steps of breath detection 728 and detection of loud breaths 730 are thus tailored to the individual, and calibrated to the sensitivity of the measuring device at that time (step 720, Signal acquisition). Loud breath sounds at night may indicate snores 760, which can occur in normal individuals exacerbated by extreme fatigue or alcohol consumption, as well as individuals with obstructive sleep apnea. Loud breaths can also indicate disturbances 758, i.e. events associated with arousals from sleep or after apnea, coded by the invention as disordered breathing (see definition and glossary of terms).

All aspects of breath detection 728 and subsequent steps of breath analysis 730-768 are tailored by the enciphered network 729. In this embodiment, the enciphered network incorporates data from other sensors in that individual to help detect each breath, e.g. oxygen waveform fluctuations, fluctuations in ECG amplitude, fluctuations in heart rate.

Step 732 detection of quiet breaths, apnea and quiet periods is the core of one preferred embodiment for sleep breathing health. Quiet periods, i.e. no sounds recorded, can be determined from step 720 including signal calibration. Separating quiet periods from apnea (i.e. quiet periods between breaths) requires high confidence in the detection of breaths. Identifying quiet breaths requires absolute cutpoints on what constitutes a breath (i.e. a database), and tailored data on what constitutes a breath in that individual under those circumstances (i.e. from the enciphered functional network 729 cross-referenced to other sensed signals). For instance, a quiet sound consistently in phase with chest movement likely relates to quiet breaths, while a quiet sound consistently out of phase/unrelated to chest movement more likely indicates non-breathing sources, which may indicate that the sound detector is too far from the individual to detect breaths. Appropriate steps will be taken, such as informing the individual to move the sound detector closer, or filtering out the sound if it is still unrelated to mechanical ventilation. Intervals between breaths (typically called apnea if >10 seconds in duration) can be related to snores, disturbances and normal breaths.

Step 734 tailors the algorithmic analysis of the invention to clinical features of that individual. In the preferred embodiment, scoring systems for sleep disordered breathing include the STOP-BANG score, which includes physical examination findings such as neck circumference, and the Epworth sleepiness scale (ESS) indicates symptoms.

Step 736 tailors the invention to signatures from other functional domains, using the enciphered functional network 729 to combine sensory signatures across functional domains. In the preferred embodiment for breathing health and disorder, several sensory signatures of breathing are combined including airflow (breathing sound files), chest movement (lung expansion), oxygenation (from skin sensors) for that individual (e.g. items 260-290 in FIG. 3). Another preferred embodiment combines signatures of brain function (e.g. nerve signatures from the scalp indicating alertness or sleep, e.g. items 210-260 in FIG. 3, FIG.4). The enciphered network is able to integrate previously stored patterns of normal and abnormal functional for that individual, and can also integrate databased patterns from other individuals for comparison purposes and/or when data from that individual is sparse.

Step 738 in FIG. 7B. outputs an index of breathing health. This index can be used to modulate the bodily task by the invention (e.g. FIGS. 5,6), to educate the individual, or to assist in clinical evaluation by a traditional (i.e. on-site face-to-face evaluation) health-care provider, online health-care provider networks, or automatic medical treatment device. In a preferred embodiment, the index of breathing health is used for education of the individual, and can be forwarded to a designated health-care provider which can include online web-based health-care provider networks.

In one preferred embodiment of the invention to monitor breathing health, the index of breathing health is provided only to the individual whose biometric data or login information matches that stored for the individual whose sound files were analyzed. These data can be provided to other designated entities (e.g. a physician's office) if designated by the individual in question. Similarly, effector responses are delivered to the individual, possibly in conjunction with confirming a match in biometric data to the stored information from that individual. This confirmation can be accomplished in hardware or software. Hardware embodiments include sensors of biometric information specific to that individual, such as a fingerprint, retinal scan, picture of the iris or unique facial features, composition of sweat, salivary composition (for sensors in the mouth), mucous composition (for sensors in the nostrils or elsewhere in the airway), sensors to analyze heart sounds, breath sounds or speech patterns. Software embodiments include spectral analyses, pattern matching analyses or correlative analyses of these sensed biometric signals to known signals from that individual. Known signals from that individual can be sensed at the time of data recording, from a prior stored event, or from a database.

FIG. 7C portrays, for a preferred embodiment of the current invention, analysis of sound files from a consumer smartphone in an individual after informed consent on an institutional review body approved study during prescribed a clinical sleep study. FIG. 7C portrays detected normal breaths, intervals between breaths and snores with no long pauses between breaths (i.e. no apnea). Such sound files may be in several formats including “.wav”. In panel 770 the sound file is checked, validated and noise eliminated (as in FIG. 7B), and represented spectrally after Fourier transform. The resulting graph shows time horizontally for 1 minute (60 seconds), the vertical scale indicates frequencies of sound at each point in time in kHz (from 0 to 20 kHz) and the intensity of color indicates amplitude at each frequency and time.

In FIG. 7C, panel 770, vertical yellow stripes represent breaths every 2-3 seconds (i.e. rate 0.33 to 0.5 Hz). Panel 771 represents these spectral bands as amplitude-time (peak/trough) sound graphs of spectral amplitude over time scaled in decibels (could be any measure of amplitude). In another embodiment, panel 771 could represent the amplitude of chest wall movement over time, plotted such as excursion at a specific point in millimeters, chest circumference in millimeters, or chest volume in milliliters. Panel 772 presents a clinical sleep study tracing (polysomnogram, PSG) in this individual, obtained simultaneously with the sound files. This PSG includes EEG channels (brain wave activity from scalp electrodes), the EMG (electromyogram), airflow channels, oxygen saturation channels and others.

Comparing panels 770, 771 and 772, analysis of sound files from the smartphone correlates well with detection of normal breaths and sleep disordered breathing from the simultaneous PSG. Item 773 shows ‘normal breaths’, identified by peak/trough amplitudes in the range of 1.5 to 4.5 dB in this case. Time periods between breaths are evident, but no apnea (>10 seconds without breaths) is seen. Item 774 shows loud sounds with amplitude >4.5 dB classified by the invention as ‘disturbances’ which correlated with disturbances on the PSG. In this case, disturbance on the PSG reflect a cough, but in other instances could indicate a snore, arousal or near arousal after an apneic or hypopneic event, or non-breathing related noises. The absence of apnea or other abnormalities (e.g. reduced oxygenation on PSG) indicates that this case does not represent a sleep breathing disorder. Amplitude ranges and cutpoints are tailored to each individual, to the distance from smartphone to patient and other factors.

FIG. 7D illustrates another case using a preferred embodiment of the invention, in which sound file analysis from a smartphone alone identified normal breaths, a period of apnea, a period of abnormal disturbance and snoring confirmed in that individual by simultaneous PSG that confirmed sleep disordered breathing. Examining FIG. 7D in detail, panel 780 from 0 to 20 seconds indicates 5 vertical colored bars (i.e. rate of 0.25 Hz), each lasting for <2 seconds when analyzed in panels 781 and 782, of amplitudes 1.5 to 4.5 dB. These bands were classified as normal breaths in this embodiment. Conversely the period from approximately 22 seconds to 45 seconds shows absence of sounds (for >10 seconds) which suggests clinically relevant apnea. Item 785 shows the time period from approximately 45 to 60 seconds showing resumption of loud breaths (amplitude >4.5 dB tailored to this individual), and closely spaced ‘clustered’ sounds of cumulative duration 4-5 seconds between 55 to 60 seconds which were classified by the invention as sound disturbance. Of note, this period corresponds in time to a clinically identified arousal event on blinded analysis of the simultaneous PSG (item 785).

FIG. 7E shows a flowchart of a preferred embodiment to detect breaths and apneas. The file is read at item 40000, and analyzed spectrally using Fast Fourier transform (item 40010). The spectrogram is analyzed for amplitude over time (item 40020), from which graph peaks and troughs are defined as in FIG. 7C (panel 771) and FIG. 7D (panels 781, 782). A windowed root-mean-square (RMS) envelope function (item 40030) smooths out fluctuations and clarifies peaks (Step 40040). This is seen by comparing panel 781 (pre-windowed RMS) to panel 782 (post-windowed RMS) in FIG. 7D. To avoid identifying low-amplitude noise variations as peaks, preferred embodiments identify peaks if >10% above baseline (item 40050). An index termed ‘prominence’ is used to identify peaks that are used as breaths (item 40060). Prominence is a mathematical function derived from topography, where prominence characterizes the height of a mountain's summit by the vertical distance between it and the lowest contour line encircling it but containing no higher summit within it. In one preferred embodiment, a prominence threshold of >0.21 is used. Such dynamic thresholds can be tailored to the individual based upon one or more of recorded patterns in that individual, recorded patterns in other individuals, patient history, population characteristics, machine learning, disease type, and other patterns. It is to be expected that all thresholds may vary and be dynamically tailored to the individual, with loudness based on proximity of the smartphone to the individual and other factors. After this step, apnea is defined if breaths are absent for a defined period of time (which is >10 seconds in this example). The final list of annotated breaths is then compiled.

FIG. 7F presents the steps of flowchart in FIG.7E in a preferred embodiment. Spectral analysis of the sound file in step 41000 produces bands of sound (colored yellow), which are subjected to peak-trough analysis (step 41010), then root-mean-square windowing (step 41020). The baseline value is then computed, and signals higher than 1.1× baseline (i.e. 10% above baseline) are identified (step 41030). This 10% value is empirical, and may be adjusted higher for noisy signals (e.g. higher baseline variations) or when signal-to-noise ratios are lower, or adjusted lower for relatively noise-free signals or when higher sensitivity is needed. The time from about 2 to 22 seconds exhibits loud breaths with several over 4.5 dB in amplitude. These sounds were consistent with loud snoring. There is then a period from 22 to 38 seconds when no breaths are identified, consistent with clinically relevant apnea (item 41070), i.e. no peaks with prominence >0.21 threshold (item 41080), or amplitude >1.5 dB. High amplitude peaks (loud sounds) then resume after about 38 seconds until the end of the tracing. Note that multiple peaks are often tagged very close together in time (item 41090), which are reconciled by selecting the one of higher amplitude. On independent blinded analysis from PSG, this patient had an apneic event with arousal corresponding to the time 22 to 38 seconds, and was diagnosed with clinically relevant obstructive sleep apnea.

FIG. 7G. shows how a preferred embodiment detects loud sounds—which are termed disturbances—and are then further analyzed (via the enciphered functional network) to classify them as loud snores or arousal events on the PSG, or noise. In step 42000 the windowed RMS envelope (e.g. item 782 in FIG. 7D, item 40030 in FIG. 7E, item 41020 in FIG. 7F) is analyzed. The signal is smoothed in step 42010, which can take place by many methods, one of which is high-order median point filter (e.g. 1000 timesteps of 1 ms each). Step 42020 repeats the peak-trough detection step, and step 42030 identifies peaks >10% of baseline (as in item 41030 in FIG.7F). The 10% threshold can be tailored to the recording and the individual. Step 42040 applies the prominence threshold >0.21, though thresholds are also tailored to the individual and may be dynamic. Step 42050 considers multiple tagged peaks within a close time interval, and identifies the largest peak. Step 42060 finds the area from this tallest peak backward and forward to the baseline, as shown in step 42110 by the shaded area. Larger areas are more likely to be abnormal loud breathing or noise. In a preferred embodiment, areas >1500 analogue-to-digital units (ADU) in dB.milliseconds are identified as disturbance (step 42070, item 42075). Panels 42080 indicates the spectral analysis, 42090 the peak trough graph and 42100 the median filtered peak trough graph, respectively. As shown in FIG. 7D (item 774), and FIG. 7F (item 785), device-detected disturbances correlate with arousals on PSG in a clinical trial.

FIG. 7H presents more detail on the area calculation to assign a disturbance sound in a preferred embodiment. Item 43000 shows the summary of peak areas for a sound file. Item 43010 indicates an example of the RMS windowed, spectral analysis of a sound file. Each of the peaks shown is analyzed for areas, as indicated by items 43020 and 43030. A threshold area of >1500 Analogue-to-digital units (dB).ms was derived empirically from a clinical trial comparing sound analysis to clinically analyzed PSG files in a derivation cohort of patients, and was then confirmed in a separate validation cohort.

FIG. 7I illustrates detection of disturbance which corresponds to noise, using the sound analysis from a smartphone in another preferred embodiment. This sound was classified as non-breathing in the simultaneous PSG, and reflected body movement and turning in bed. Item 790 shows a spectrogram of sound with yellow bands that do not plausibly represent breaths, i.e. no yellow bands at 0.2 to 0.5 Hz, bands of duration <2 seconds, and most amplitudes <1.5 dB. Item 791 shows this more clearly. Item 793 highlights the period from approximately 15 to 25 seconds with a broad (>5 seconds) low amplitude (<1.5 B) envelope (panel 791) which correlates with body movement on the PSG (panel 792). Panel 794 shows the period from 37 to 45 seconds shows a broad (5-10 seconds) high amplitude (>4.5 B) envelope which correlates in time with body movement on blinded analysis of the simultaneous PSG (Item 792). Notably, breathing continued throughout this period (see flow channel on PSG, item 792) indicating that the sound file does not indicate breaths. This was a case of the smartphone being too far from the face of the individual to detect breathing, but instead picking up body movement. This time segment of the file was discarded from analysis.

FIG. 7J shows how a preferred embodiment of the invention analyzes quiet periods (i.e. no sound) versus apnea in between breaths. Item 44000 shows a sound file spectrogram with no clear periodic activity. Item 44010 indicates multiple very closely spaced peaks, each of which has a very low dynamic range. The preferred embodiment filters out these signals because they are not >1.1× baseline, and have a low dynamic range. This file corresponds to a smartphone that is too far from the face of the individual to detect breathing. Item 44020 indicates a similar file, with two potential bands on the spectrogram at approximately 48 and 52 seconds. Item 44030 indicates that these bands meet the criteria outlined above for breaths. The logic of the enciphered functional network will then compare these bands with known breath periods, such as after or before this segment, to determine if these are breaths following a long apneic period, or if these bands are noise in a period when breaths are not captured.

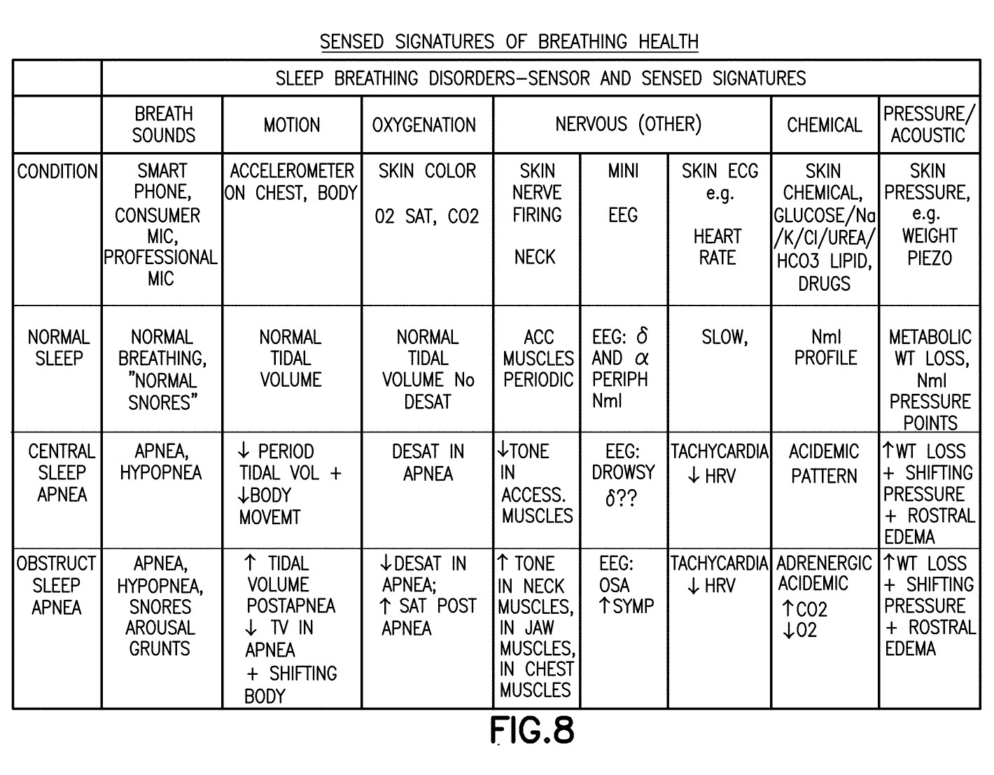

FIG. 8 is a preferred embodiment of sensed signatures in sleep-breathing disorders. As is typical for many bodily tasks, sleep-disordered breathing impacts multiple nervous and non-nervous system functional domains. Of all of the domains that can be sensed, not all domains need to be sensed in every patient. The actual sensed domains (and hence sensors) used in an embodiment can be tailored to that individual and practical considerations. As seen in FIG. 8, sensor types can include but are not limited to microphones in a smartphone, skin impedance, other electrical sensors (nerve firing in the periphery and on the scalp, and heart rate), temperature, chemical sensors, optical sensors of skin color (that can detect oxygen saturation of peripheral blood), motion sensors and pressure sensors.

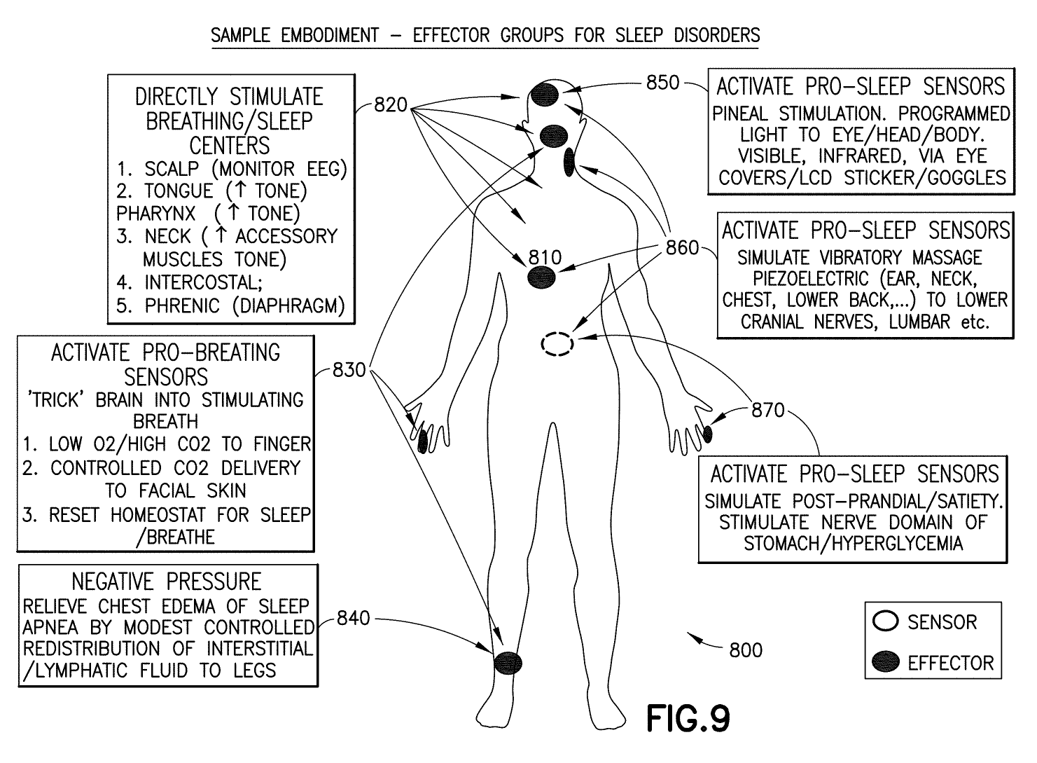

FIG. 9 indicates sample embodiments for effectors of sleep-disordered breathing by the enciphered functional network. These are provided by way of example and in no way limit the scope of effectors or treatment options that the invention can provide for breathing health or other bodily functions. The body 800 is interfaced with effector devices 810, tailored to each modality. For a preferred embodiment of sleep apnea 820 of the central type, effectors may directly stimulate breathing centers including the brain (via low energy scalp stimulation), accessory muscles in the neck and the diaphragm. For central sleep apnea, the invention aims to activate pro-breathing centers, causing the brain to signal higher breathing rates by direct stimulation of scalp regions, or by stimulating sensors of low oxygenation/high carboxyhemoglobin in the finger, by providing CO₂ or equivalent index of low breathing to regions of the periphery that are not harmful. In a preferred embodiment of the invention for obstructive sleep apnea, effectors may directly stimulate pharyngeal and neck muscles to maintain tone and prevent obstruction. Direct stimulation of pro-sleep centers by other methods 850 include stimulation through light exposure of the appropriate wavelength in the visible and infrared spectra. This may stimulate the pineal of other sleep-wake centers in the nervous system. Light can be provided in patterns that are specific to each individual and can be learned by the device. Other pro-sleep sensors include activation of vibratory sensors 860 to mimic the somnorific impact of massage, or stimulation of post-prandial satiety sensors 870 including stimulating peripheral skin sensors of abdominal fullness or hyperglycemia. For both central and obstructive forms of sleep apnea, there is evidence of chest edema (water accumulation) which can be measured as an increased rostral-to-peripheral ratio of skin impedance (FIG. 7). Accordingly, controlled negative pressure in the lower extremities 840 can be used to reverse rostral fluid accumulation. Other specific stimuli can also be provided as familiar to one skilled in the art of sleep disorders, and can be added to the infrastructure of the invention as new modalities and sensed signatures are developed.

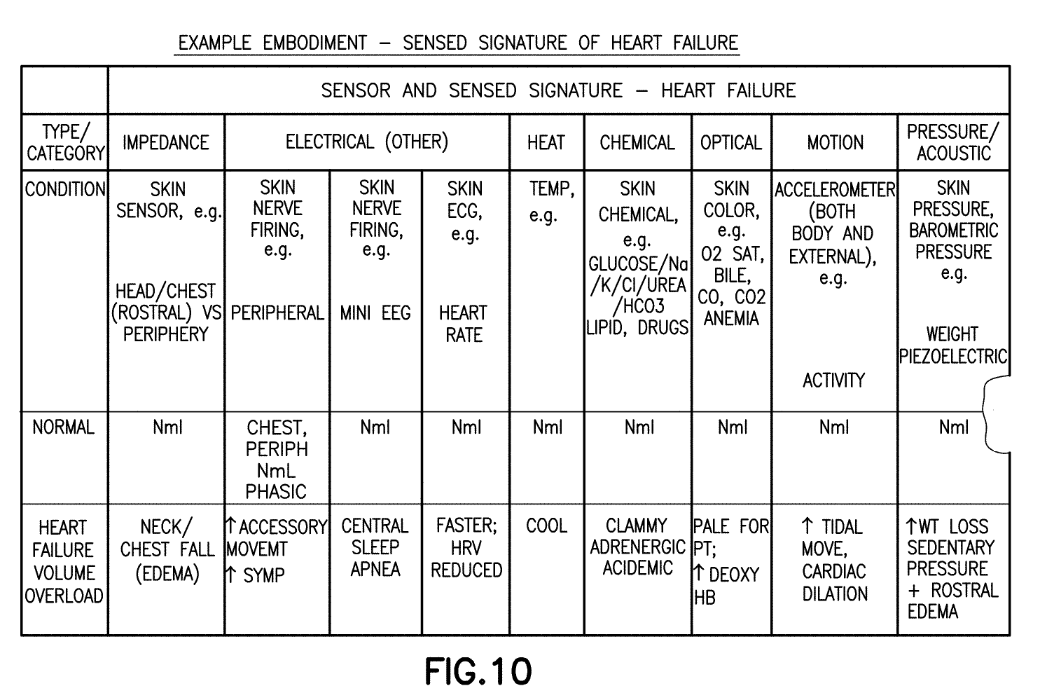

FIG. 10 indicates an example embodiment of sensed signatures for heart failure. As is typical for many bodily tasks, heart failure impacts multiple nervous and non-nervous system functional domains. While the invention may sense any domain, not all domains need to be sensed in every individual, and the actual sensed domains (and hence sensors) can be tailored to a given individual and practical considerations. As seen in FIG. 10, sensor types can include but are not limited to weight sensors (FIG. 7A, item 710) in dedicated scales, in a smart car seat, in shoes, in the floor of a building. Other sensors for heart failure include, skin impedance, electrical sensors to measure nerve firing in the periphery to measure sympathetic tone, and on the scalp to measure EEG, sensors of heart rate, temperature, chemical sensors, optical sensors of skin color (that can detect oxygen saturation of peripheral blood), motion sensors and pressure sensors.

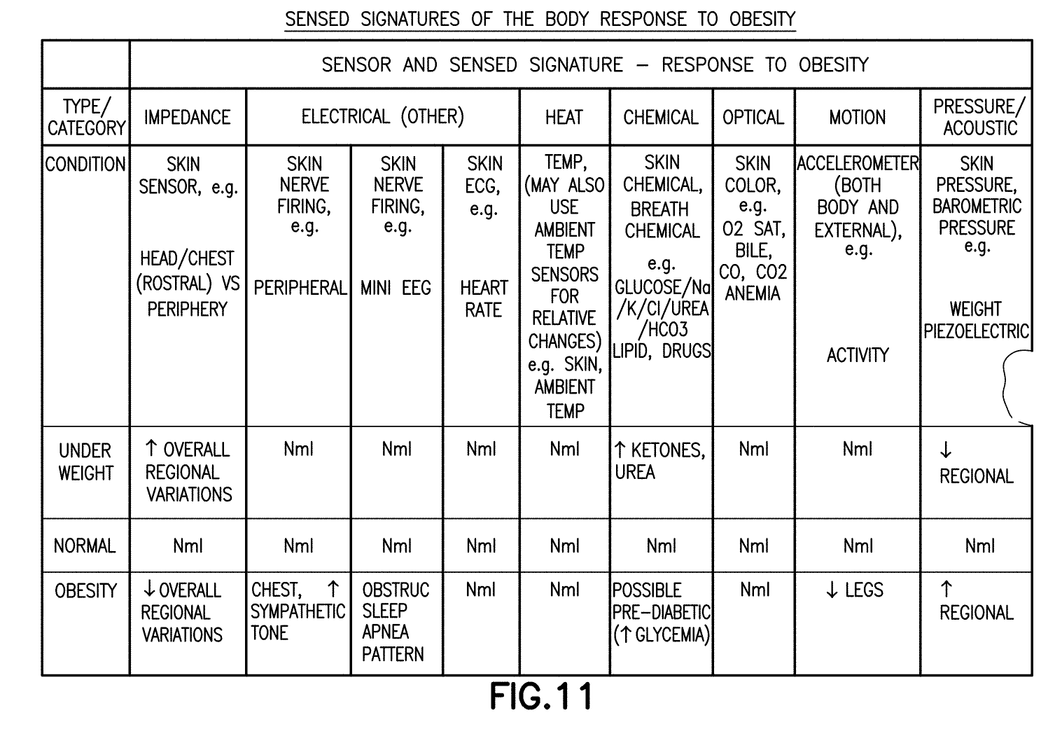

FIG. 11 indicates an example embodiment of sensed signatures of response to obesity. As typical for many bodily tasks, obesity impacts multiple nervous and non-nervous system functional domains. While the invention can sense any domain, not all domains need to be sensed in every individual, and the actual sensed domain (and hence sensors) can be tailored to a given individual and practical considerations. As seen in FIG. 11, sensor types can include but are not limited to skin impedance, other electrical sensors (nerve firing in the periphery and on the scalp, and heart rate), temperature, chemical sensors, optical sensors of skin color (that can detect oxygen saturation of peripheral blood), motion sensors and pressure sensors.

FIG. 12 shows an example of sensed signatures for other conditions. One example is for chronic obstructive pulmonary disease which, as is typical for diseases with many complex bodily tasks, impacts multiple nervous and non-nervous system functional domains. While the invention can sense any domain, not all domains need to be sensed in every individual, and the actual sensed domains (and hence sensors) can be tailored to a given individual and practical considerations. As seen in FIG. 12, sensor types can include but are not limited to skin impedance, other electrical sensors (nerve firing in the periphery and on the scalp, and heart rate), temperature, chemical sensors, optical sensors of skin color (that can detect oxygen saturation of peripheral blood), motion sensors and pressure sensors.

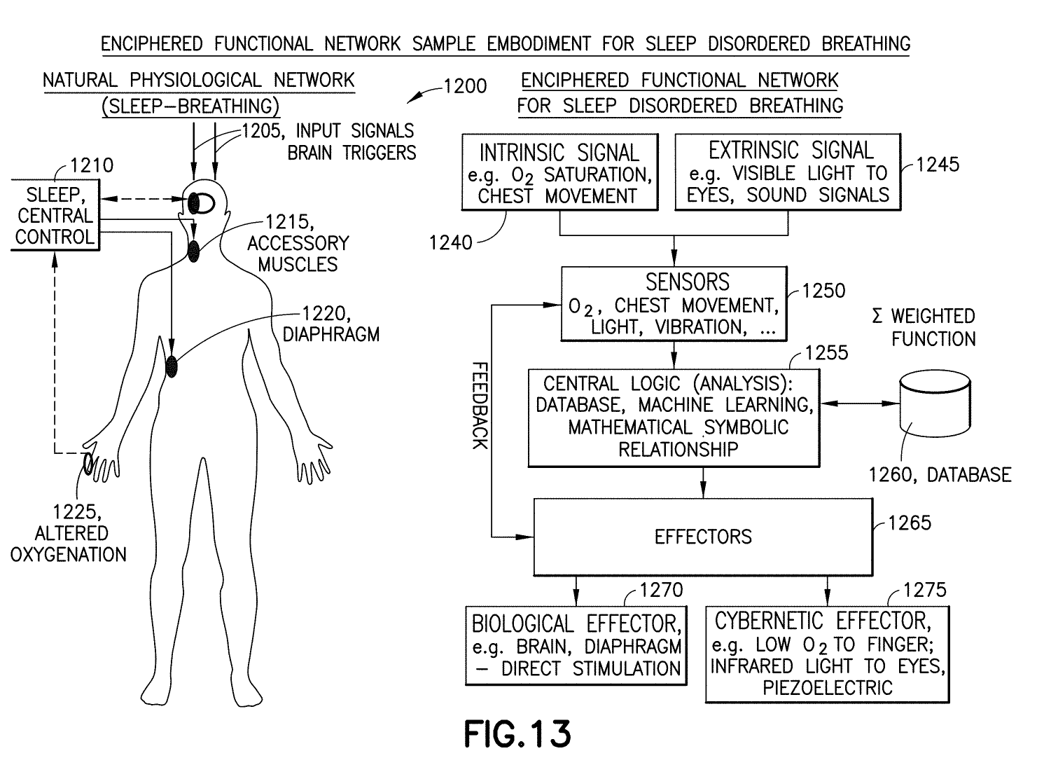

FIG. 13 summarizes the invention, a computerized representation of a complex body task, paired to biological and artificial sensors(cybernetic), and biological and artificial (cybernetic) effectors. The enciphered functional network is trained for specific bodily tasks. In the simplest case, sensed and effector functions are natural physiological functions, such as sensing a painful stimulus from the leg and moving the leg away. In complex embodiments, the invention has the ability to enhance normal function (performance enhancement), enhance impaired function (e.g., sleep-disordered breathing) or treat a disease or in cases where normal function cannot be manifest (e.g., in warfare or other situations of constraint).

More specifically, FIG. 13 outlines the preferred embodiment of an enciphered network for sleep-disordered breathing. The left panel shows the actual physiology measured for sleep disordered breathing, while the right panel shows the computerized representation of the enciphered functional network.

In measuring the actual physiology of sleep-disordered breathing in an individual 1200, biological signals are sensed 1205. These include biological signals of control regions 1210 including activation of the amydala and other parts of the limbic system that control alertness, wakefulness and relate to sleep. These signals have scalp representations that can be detected by skin nerve sensors, but can also be detected by medical devices such as the BOLD signal from functional magnetic resonance imaging, or metabolic images from positron emission tomography in medical applications. Physiologically, sleep is also triggered from intrinsic but natural signals such as darkness, sound (e.g., soothing music or the sound of waves), tactile sense (e.g., massage of parts of the body). The intrinsic sleep control regions of the brain 1210 then integrate these inputs with sensors related to breathing including low oxygenation, measureable in the fingertips 1225, that stimulates breathing, and stimulation of the diaphragm 1220 to enable ventilation of the lungs.

The schematic shown in the left panel of FIG. 13 is a simplified view of sleep-related-breathing, but it illustrates how a series of sensors and effectors are integrated by the biological control regions. Other sensors and effectors can be involved at other times, and can be measured in connection with the sleep-related breathing. That additional sensed signals can be added and will be adaptively integrated by the enciphered network is a strength of this invention.

The right panel of FIG. 13 depicts the enciphered network for sleep-disordered breathing in parallel. This also has sensors, control logic and effectors, but these are a combination of biological and engineered (artificial) components. Sensors can detect intrinsic signals 1240 (such as oxygen saturation) or extrinsic signals 1245 (such as the presence, intensity and patterns of visible light). A sensor matrix 1250 then combines these biological and non-biological signals either separately or by multiplexing them, e.g., using a weighted function. The computational logic 1255 is the central processor of the enciphered functional network.

The computational element 1255 uses symbolic relationships between sensed signals and biological function (e.g., elements 250-290 in FIG. 1). It is linked to a database 1260 to store multiple states for this individual person as training datasets for machine learning (i.e., fuzzy logic, artificial intelligence) in order to learn normal sleep patterns and breathing from disordered ones (elements 250 versus 290 in FIG. 2). This is then mapped to effectors 1265 that can be biological, such as brain regions (related to control regions 1210 and unrelated to control regions 1210) as well as muscles (the diaphragm 1220 as well as other muscles that are less notable but also involved in sleep such as the levator labii superioris alaeque nasi muscles). Effectors can also be cybernetic 1275, in that they interface artificially engineered devices with the body. For instance, a peripheral low oxygen state can be mimicked by small wearable chambers (“treatment gloves”) surrounding one or more fingers that will stimulate breathing from intrinsic sleep-brain control centers (control regions 1210). Similarly, appropriate learned patterns of light or of vibratory stimuli can be applied using appropriate devices, to stimulate sleep-breathing patterns learned from normal states and stored on the database 1260.

The analysis engine of the enciphered network in FIG. 13 is a symbolic relationship which may be mathematical. This mathematical relationship can be used for mathematical weighting for diagnosis tailoring. Such weighting can be constant and/or adaptive based on learning input streams of sensed signatures. Such weighting can be performed by various methods including but not limited to stochastic methods, correlation methods, calculus based approaches, geometric based methods and spectral methods. The mathematical relationship uses functional relationships between sensed signatures and variations in the body task for that individual—and is not primarily based on theoretical or anticipated relationships. Thus, it may not follow “classical” physiology. For instance, in some patients shoulder pain is associated with heart problems and thus can be part of the sensed signature of heart pain (‘angina’) in such individuals even though shoulder nerves play little or no part in the pathophysiology of heart blood supply. In another example, pain in the leg may elevate nerve activity elsewhere in the body, such that painful leg disorders may be detected using sensors located elsewhere e.g. in more convenient body locations. The functional relationship adapts to sensed signatures and health states tailored to the individual, and such tailoring is based on and may use deterministic (e.g., rule based) or learned methods as outlined throughout this Specification.

In the simplest case, the symbolic relationship in the enciphered network is a matrix in which a signal X causes a function Y; for instance, a noxious stimulus such as pain sensed by a sensor/sensory nerve in the leg (X) causes activity in a motor nerve causing withdrawal of that leg (Y). This function is not represented in the device based upon a detailed neurophysiological representation of leg sensation (in the primary somatosensory cortex, in the post-central gyrus), or the precise nerves that control the leg. Instead, this function is mapped empirically—sensation on any nerve associated with the painful stimulus can result in actions leading to leg withdrawal.

The advantage of this approach is that it can analyze the multiple effects of a particular stimulus. For instance, an acute painful stimulus often produces activation on nerves remote from the original site of stimulation. Hence, pain in the leg, that may be inaccessible, may be detected from nerve activity quite distant from the sensation such as the chest wall, that may be more accessible.

In FIG. 13, generalizing from the example for sleep breathing, sensing is processed and results in output to an effector. For instance, the sensed noxious stimulus can produce an effector function to move the leg, or control a device to administer a pain killing medication or therapy. In other examples that will be discussed below, the stimulus can move a prosthetic limb or alter biological function.

Moreover, FIG. 13 shows that the enciphered network determines precise action by defining interactions with the device or bodily function. This is a programmed function, depending upon the desired functionality of the invention. This then produces a real output requiring application of energy that results in interaction with the device or a bodily function.

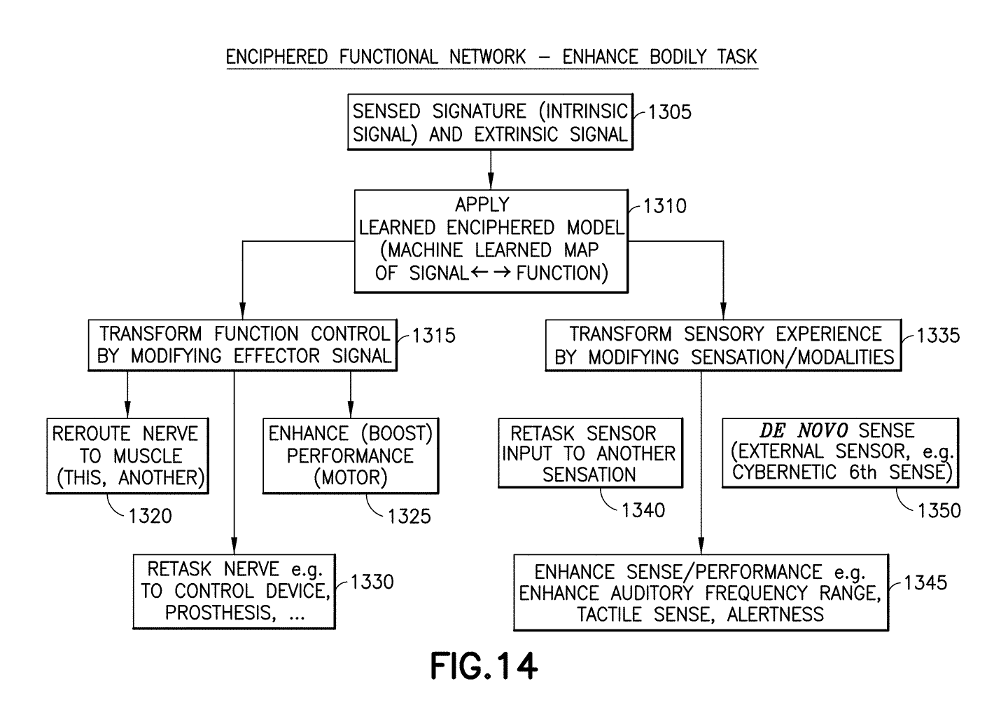

FIG. 14 illustrates a preferred mode of action of the invention to provide computational enhancement of the bodily function via the enciphered functional network. The flowchart for the invention senses signatures for a given bodily function 1305, comprising biological signals (e.g., breathing rate, finger oxygenation) or extrinsic signals (e.g., tissue impedance indicating volume load, emitted infrared indicating temperature, or carbon dioxide concentrations in exhaled air indicating the efficiency of breathing).

Item 1310 applies the symbolic model of the enciphered network for an individual, as identified in FIG. 8 to map sensed signals to a bodily function based on practical measurable signatures rather than classical, detailed physiology mapping that may be ill-defined, rapidly changing and inaccessible to measurement.

As described above, the symbolic model uses machine learning to map sensor input to normal and abnormal function of that bodily functionality. This comprises training sets of different patterns for that specific individual, making the output both personalized and continuously adaptive.

In FIG. 14, step 1315 transforms an effector (motor) function, i.e., controlled by an existing motor nerve. In step 1320, the motor nerve signal is “re-routed” to control a prosthetic device or another muscle group. For instance, in the case of an amputee, the signature of motor nerve output to the leg may be detected from the skin above the amputation site. The range of sensed nerve activity on the skin may typically be 7-15 Hz (depending on the precise nerve). Sensing these signals, and mapping them to specific movement of a prosthetic limb may enable control of the limb. This control may require subsequent training—for instance, behavioral training in which the individual attempts to flex the amputated limb, and detecting the skin signals as those that will flex the prosthetic limb in that person. Similar personalized mapping is used to train other motions of the prosthesis. In this instance, the invention is one embodiment of a personalized “enciphered nervous system”.

In FIG. 14, step 1325 is another embodiment—to enhance performance of this body function. Instead of expending the energy required to move a finger, the enciphered network can sense sub-threshold activity of the motor nerve and “boost” the signal to move the finger 1325. This is useful for individuals with nerve degeneration, those with musculoskeletal disorders or those under some form of sedation who would normally not be able to communicate via this finger.

Furthermore, the invention can 1325 artificially generate signals needed to stimulate the muscle. Since the frequency and amplitude of nerve activity that controls a muscle lies within a range for each individual, the enciphered network can simulate the nerve activity controlling the quadriceps femoris muscle and deliver it programmatically to regions of the skin associated with contraction and relaxation of that muscle for that individual (part of the functional domain). This can be used when the nerve is degenerated or anesthetized (for instance, to prevent pressure ulcers in patients on prolonged ventilation). It can also be used for performance enhancement—for instance, to perform isometric exercises during rest or sleep to prevent or reverse muscle atrophy, or to improve muscle function or increase metabolic rate to lose weight.

In FIG. 14, step 1330 is another embodiment of the invention—to retask biological motor activity. In this case, it is directed to control an artificial device. This cybernetic application is further developed in FIG. 14. In FIG. 13, instead of actually moving a finger to control a remote control unit for an electronic device, nerve activity below the threshold of actually moving that finger will control the device. This enables functionality without expending as much biological energy, and also in individuals who have lost biological function or are constrained and unable to perform that motor function (e.g., in a military situation). Sensors on the finger detect this subthreshold motor nerve activity (e.g., of lower amplitude than biologically required to move the finger), and the enciphered network converts this to signals that represent play, pause, rewind or other functions and transmits them to control the remote control unit. This may be for a consumer device. Clearly, this function can be extended to training an individual to move a portion of the face to represent the “play” function, and having a sensor transduce this function, and similarly for other surrogate regions of the body and retasked functions.

In FIG. 14, step 1335 is a distinct embodiment that transforms sensed signals. Step 1340 retasks the sensed signal. For instance, sensation of a specific smell that is trained over time, can elicit a different response or control a device. Step 1345 improves performance, augmenting biological outside of normally sensed ranges. For instance, sensing signals in the “inaudible to humans” frequency range, transducing the signal to the audible range, and transmitting it via vibration (bony conduction) to auditory regions of the brain (auditory cortex) could be used for private communication, encryption, recreation or other purposes. Medically, this invention could be used to treat hearing loss. This same invention with sensors of vibration could be used to compensate for loss of this sensation in diseases such as peripheral neuropathy, by transmitting this sensation to an intact sensation in a nearby or remote part of the body.

Another embodiment of performance improvement (step 1345) is to increase alertness. Stimulation of the scalp in the temporal region and other function-specific zones can increase brain activity in these regions. The invention tailors stimulation to the enciphered representation of awakeness (i.e., alertness). As a corollary, drowsiness can be detected by the enciphered network and used in a feedback loop to trigger low intensity stimulation by a cutaneous device elsewhere on the body. This has several applications, including detecting and trying to prevent drowsiness while driving, in the intensive care unit during pre-comatose states or during drug-overdoses, as a monitor for excessive alcohol or medication ingestion, or during excessive fatigue states, e.g., in the military.

Sensors can detect alertness versus drowsiness from large groups of neurons using electroencephalography (EEG) over a wide range of frequencies. EEG signals have a broad spectral content but exhibit specific oscillatory frequencies. The alpha activity band (8-13 Hz) can be detected from the occipital lobe (or from electrodes placed over the occipital region of the scalp) during relaxed wakefulness and increase when the eyes close. The delta band is 1-4 Hz, theta from 4-8 Hz, beta from 13-30 Hz and gamma from 30-70 Hz. Faster EEG frequencies are linked to thought (cognitive processing) and alertness, and EEG signals slow during sleep and during drowsiness states such as coma and intoxication. Alertness vs drowsiness can be potentially detected via other sensors including, but not limited to, visual (e.g. eye tracking or head movement), auditory (e.g. change in speech or breathing sound patterns), and electrical (e.g. ECG measures for autonomic function). The enciphered functional network can integrate these additional sensed data and can assess if they provide useful sensed signatures of normal or abnormal function of that task in that individual.

In FIG. 14, step 1350 is a function detecting and/or forming a de novo function. One example is creating a cybernetic “sixth sense”—that is, adding to the 5 biological senses using artificial sensors to detect an extended set of stimuli. The set of sensors is nearly infinite, but includes several of particular relevance to the field of industrial or military use, including sensors for alpha or beta-radiation. Once sensed, the enciphered network can transduce this signal to an existing sense, such as vibration delivered through a skin patch to a relatively unused skin region, e.g., lower back. A combat soldier exposed to alpha or beta particles will now “feel” radiation as a programmable/trainable set of vibrations in his lower back. Similarly, sensors for carbon monoxide or other respiratory hazards could be transduced as “sixth senses” into—for instance—low frequency vibration on the nostril. This approach is far more efficient than a visual readout or other existing devices—because they use the enciphered network to essentially reprogram the natural nervous system for these functions.

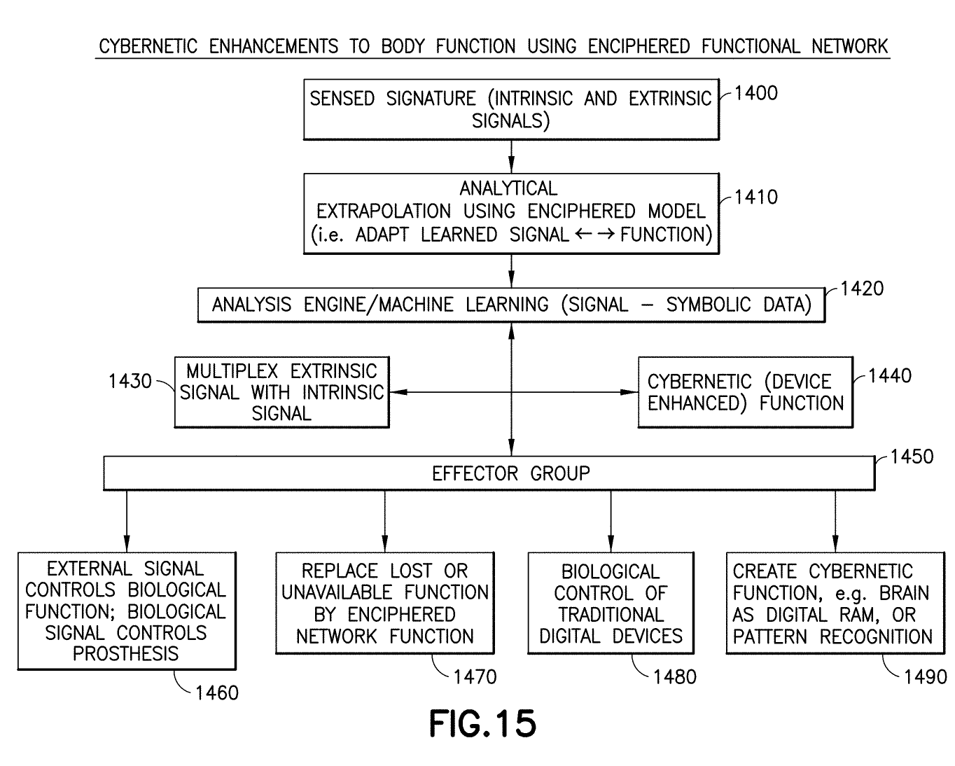

FIG. 15 generalizes cybernetic enhancement of body function using the enciphered network. This is a further application beyond the use of intrinsic biological signals. One application is to apply purposeful interventions when natural body functions are constrained, e.g., a soldier can use a finger to activate a device if his/her foot cannot activate a pedal due to an obstacle, or, in an amputee, interfacing a robotic arm to specific nerve fibers that formerly controlled the biological arm.

FIG. 15 is an embodiment in which intrinsic biological signals and extrinsic non-biological signals are sensed (step 1400). The enciphered network does not simply map learned function to sensed signals, but instead extrapolates from learned functions to create novel function 1410. The enciphered representation of the body function to sensed signals is extended to a personalized network in step 1420 via machine learning. This involves a series of steps, including 1430 multiplexing or otherwise combining intrinsic with extrinsic signals, to programmatically modify external signals in a personalized fashion. Signal multiplexing is performed to achieve the desired function 1440 that may be storage of non biological information (e.g., word processing documents, images) in the patient's brain, i.e., using biological storage as digital memory, and so on. Signals can be combined based on data from this person alone, from a database of multiple individuals (e.g., item 1260 in FIG. 12), or by a technique such as crowd-sourcing in which information from multiple persons is integrated to train the enciphered network. Data from multiple persons could be combined in a formal database, or by applying machine learning to the wider set of sensed signals and biological outputs between individuals (not just for one individual).

Step 1450 in FIG. 15 shows the effector layer, the interface between the output of the enciphered network for a designed cybernetic function and a series of biological (e.g., motor nerve, muscle) or external (e.g., prosthetic limb, computer) effector devices.

Several embodiments exist. In step 1460, the invention uses a biological signal to control an external device (e.g., motor nerve control of a prosthetic limb), or an external signal to control a biological function (e.g., external signal stimulation of a skeletal muscle). As described, skeletal muscle is typically stimulated by nerve activity at a frequency of 7-15 Hz (varying with precise nerve distribution, see Dorfman et al. Electroencephalography and Clinical Neurophysiology, 1989; 73: 215-224). Such external stimulation can improve muscle strength by stimulating it, and would enable performance improvement of, e.g., programmable improvement in leg muscle function. Another example is to treat central sleep apnea, using an external sensor of oxygen desaturation (“desat”) to activate a device that stimulates the phrenic nerve and hence the diaphragm. This may have substantial clinical implications.

FIG. 15 step 1470 shows an embodiment in which the invention replaces a biologically lost or unavailable function in that individual with function from the enciphered network. This is an extension of boosting performance in FIG. 14 (step 1325). For instance, the unavailable function of hearing outside the normal 20 Hz to 20 KHz range can be provided using external sensors and the signal transduced to the audible frequency range (e.g., vibrations delivered via bone conduction using a device placed near the mastoid processes, e.g., attached to the side-arms of eyeglasses, patch attached to head with vibration sensor) or to another sensible modality (e.g., vibration on the arm). In an individual with hearing loss, the sensed signal will lie within the normal but compromised auditory range for this individual.

In FIG. 15 step 1480, the invention enables biological control of a computer. An example of this function is to provide an intelligent control framework for an infusion pump. For instance, glucose control is not determined simply by the reaction of the pancreas and other sensing regions to plasma glucose. Instead, higher brain centers that control activities of daily living anticipate actions such as imminent exercise or stress, and produce increased heart rate and a hormonal surge (e.g., adrenaline, epinephrine) that in turn increases blood glucose. Current glucose infusion pumps actually cannot mimic such higher cognitive input, and instead wait for drops in glucose from metabolic demands before infusing glucose. Such devices will always lag behind ideal physiological control and will produce suboptimal performance.

In FIG. 15 step 1490, the invention can provide de novo functionality. This exploits the full potential of the enciphered functional network, in this case for the nervous system, and extends beyond sensory or motor performance improvement in steps 1325 (motor) or 1345 (sensory).

In FIG. 15 step 1490, novel functionality can be provided for motor function (i.e., previously unavailable movements) or sensory function (i.e., a cybernetic 6^{th} sense). A large proportion of cerebral processing power is dormant at any given time, but may be activated subconsciously during daily activity (e.g., daydreaming). The enciphered network can access some of this brain capacity to use the biological nervous system as a computer. One task for which the human brain/nervous system is particularly adept is pattern recognition. Recognition of faces, spatial patterns and other complex datasets is performed by people far better than by artificial computers. The selected example trains the individual to detect the pattern via repeated overt or subclinical exposure to an image. The biological response to this image (symbolic representation) is detected by sensors on the temporal or frontal scalp. Again, this is empirical—the primary memory encoding regions do not have to be identified or mapped, and it is sufficient to sense a secondarily activated region of the brain/scalp. Once this is accomplished, detection of the pattern or a similar pattern will subconsciously trigger the response that can be sensed and coded as a “1” or “0” to control a device (e.g., a pattern classifier computer) or cause a certain function —such as to trigger an alarm if this is a dangerous pattern/image.

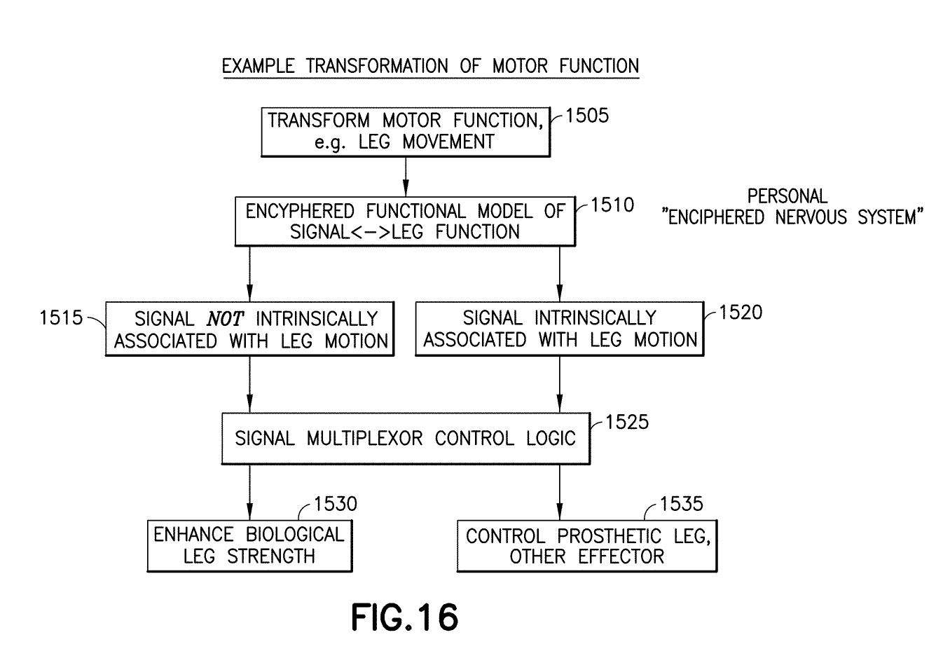

FIG. 16 illustrates an embodiment of motor function controlled by the enciphered network. The Flowchart in FIG. 16 provides a preferred embodiment to transform leg movement. A symbolic model is to link motor nerve function, sensed at a signature of the primary motor region (scalp, near the superior portion of the contralateral precentral gyrus) or a secondary region, with a plurality of leg motions in step 1510. Once done, functional mapping can be reprogrammed using external sensed signals (step 1515) including those not normally associated with leg function. An example would be for motion in an index finger to control the leg movement, in patients with leg disease or soldiers who cannot move their leg in a certain task. Functional mapping can also use the existing signal (step 1520).

In step 1525, a signal multiplexor links the intrinsic or extrinsic signals in order to control the desired programmed function. In step 1530, this is achieved to enhance biological leg function (e.g., via cutaneous/direct electrical stimulation as described). In step 1535, this is performed to control a prosthetic limb.

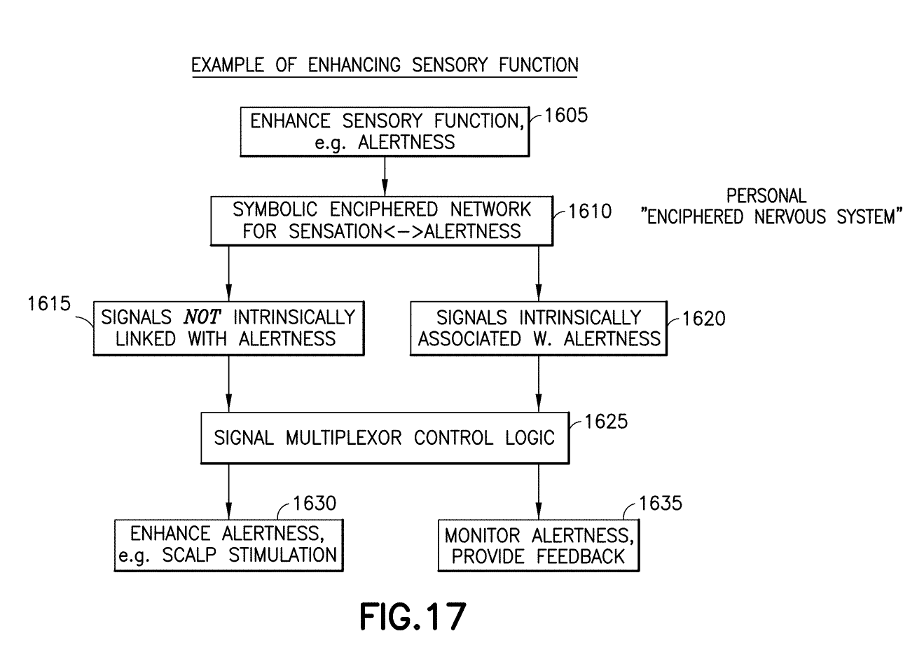

FIG. 17 shows an embodiment of enhancing sensory function via the enciphered network. FIG. 17 is an embodiment for enhancing alertness. A symbolic model is created in step 1610 using a signature of sensed scalp nerve activity, e.g., from the temporal region that is empirically associated with alertness. Functional mapping is reprogrammed using intrinsic sensed signatures (step 1615) or signals not normally associated with alertness (e.g., a specific auditory sensed frequency), or the existing scalp signal (step 1620). In step 1625, a multiplexor links the intrinsic and extrinsic signals with an effector to achieve the desired function—electrical stimulation of the scalp to increase alertness (step 1630). Step 1635 provides an alertness monitor to alarm or produce the desired function, and that can detect and try to avoid drowsiness or coma, such as during driving, on the battlefield or from toxin ingestion.

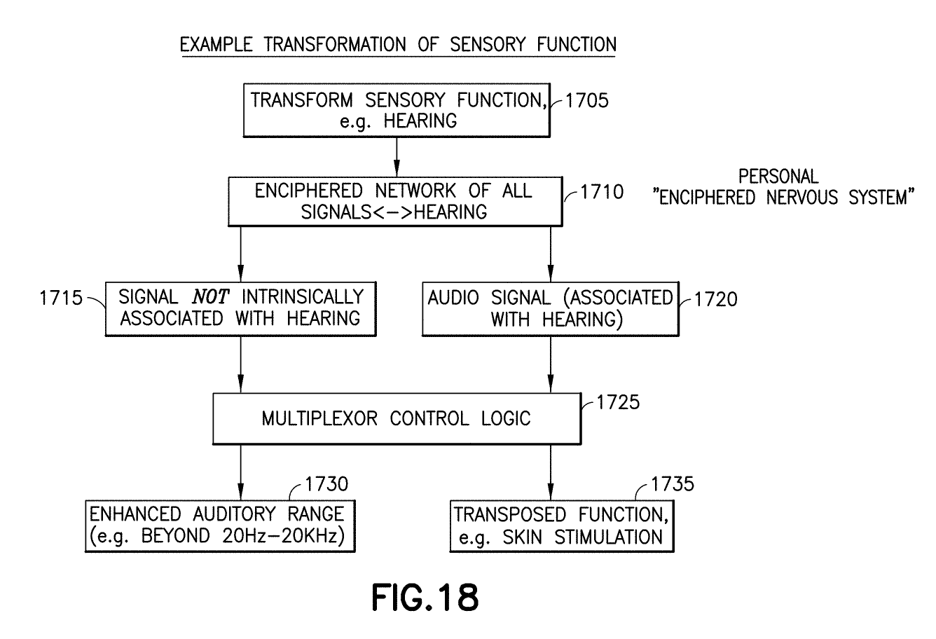

FIG. 18 depicts an embodiment of the invention to transform sensory function. FIG. 18 is a flowchart of an embodiment to enhance sensory performance—in this case hearing. Step 1710 is the symbolic representation of sensed signals from a readily accessible sensor of the signature near the ear, as well as secondarily associated skin regions. Step 1715 uses sensors to detect signatures of frequencies outside the normally sensed frequency spectrum. Step 1720 uses a signal normally associated with hearing. Step 1725 uses a multiplexor and control logic to transduce the signal to the audible range (step 1730), transmitted via vibration (bony conduction) to the hearing regions of the brain (cochlear nerve/auditory cortex) using a device that could be used for private communication, encryption, recreational or other purposes. Medically, this invention has application as a sophisticated hearing aid. This same invention with vibration sensors compensates for loss of this sensation in diseases such as peripheral neuropathy, by transmitting this sensation to an intact sensation in a different part of the body. At 1735, the multiplexor transduces this signal to a different “surrogate” sensation, e.g., skin stimulation.

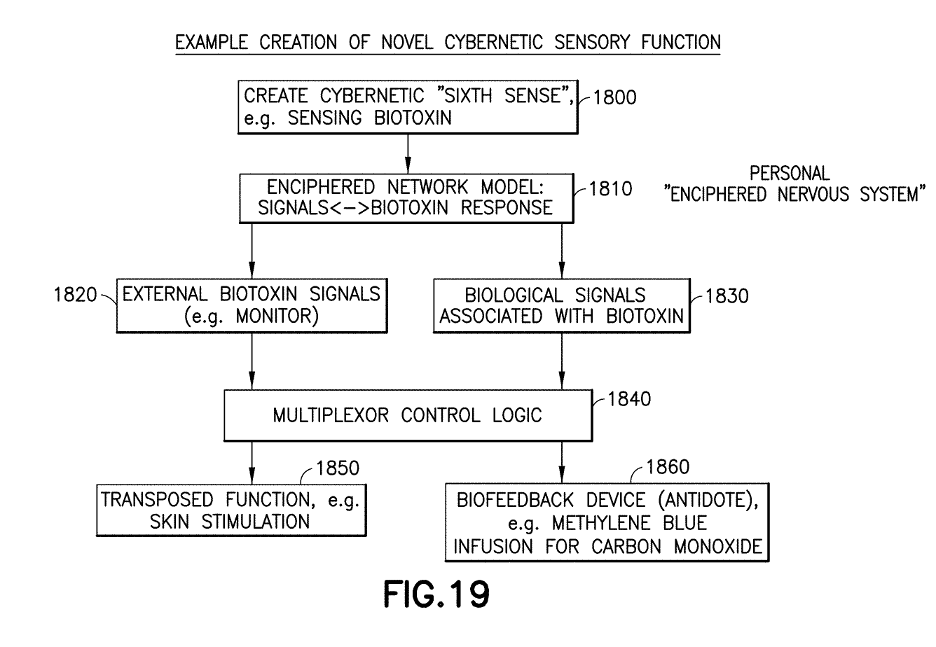

FIG. 19 shows an embodiment to create novel “cybernetic” sensory functions. FIG. 19 is a flowchart of an embodiment to create a cybernetic “sixth sense” (e.g., sensing a biotoxin). The invention summarized in FIG. 19 incorporates information associated with the example of sensing carbon monoxide. Specific sensed signals cause damage, to calibrate sensing and delivery of therapy functions. For instance, exposure to carbon monoxide is dangerous, yet this toxin is often undetected. Federal agencies in the U.S. such as OSHA put a highest limit on long-term workplace exposure levels of 50 ppm, with a “ceiling” of 100 ppm. Exposures of 800 ppm (0.08%) lead to dizziness, nausea, and convulsions within 45 min, with the individual becoming insensible within 2 hours. Clearly, an invention to detect this toxin early and cause biofeedback through the enciphered nervous system may have extremely practical implications in industrial environments. Other nomograms can be developed to identify thresholds for “safe” versus “actionable” exposure to various stimuli including but not limited to chemicals, biological toxins, radiation, electrical stimuli, visual stimuli and auditory stimuli.

The invention summarized in FIG. 19 can also be used to create novel human functionality, by using the enciphered network to pair sensed biological or external signals to any programmed biological or external device. It thus forms an embodiment of a cybernetic nervous system operating in parallel with the body's natural nervous system. The extent to which these nervous systems are parallel or integrated will depend upon the extent to which sensed signals are multiplexed and effector “control” signals are combined. Examples are discussed below.

The invention outlined in FIG. 19 thus provides hitherto unavailable programmatic control of plasticity—that is, actually observed at some level on a regular basis in normal life. In the realm of sensory physiology, training can enable an individual to perceive a sensation that was previously present but not registered/recognized. Examples include musical training to detect tonality, or combat training to detect subtle sounds or visual cues. In the realm of motor control, physical training can enable an individual to use muscle groups that were previously unused. In the realm of disease, normal “healing functions” cause healthy regions of the central nervous system to take over functions now lost due to a stroke (cortical plasticity), or unaffected peripheral nerves to take over functions of a nerve lost due to trauma or neuropathy (expansion/plasticity of peripheral dermatomes).

The current invention extends known interventions based upon cortical plasticity. For instance, it is known that the dermatomal distribution of a functioning peripheral nerve expands when an adjacent distribution is served by a diseased nerve. In other words, the same function can now be served by different regions of the central or peripheral nervous system.

The invention also substantially extends normal plasticity—by programming desired and directed regions of the body to sense and effect functions normally reserved for other regions of the body that are currently inaccessible (e.g., in military combat) or unavailable (e.g., due to disease).

The invention also substantially advances normal plasticity by integrating external sensors (e.g., for normally inaudible sound frequencies or sensations) or devices (e.g., prosthetic limbs, other electronic devices) into the ENS.

FIG. 19 may also include embodiments for enhancing sensory alertness. The steps are analogous to the prior examples. The symbolic model of scalp sensed nerve activity, e.g., in the temporal region is empirically associated with varying alertness levels (self-reported or monitored) in step 1710. This functional mapping is reprogrammed using external sensed signals (step 1715) or signals not normally associated with alertness (e.g., a specific auditory sensed frequency), or the existing scalp signal (step 1720). In step 1725 a signal multiplexor mathematically associates the non-associated or associated signals to program the desired function—electrical stimulation of the scalp to increase alertness (step 1730). Step 1735 provides an alertness monitor that can provide an alarm or actually result in stimulated function (to close the artificial/cybernetic feedback loop in the enciphered nervous system) to detect and try to avoid drowsiness, coma or toxin ingestion.

FIG. 19 depicts an embodiment to use the ENS to integrate functionality that does not exist in nature into a personalized biofeedback loop—in this case, detecting a toxin. Examples include inhalation of carbon monoxide, a toxic gas that is colorless, odorless, tasteless, and initially non-irritating, that is very difficult for people to detect. Another example is exposure to a biotoxin, that may not be sensed until symptoms and signs of a disease occur hours, days weeks later. The inventive approach to provide a “sixth sense” (step 1800) is cybernetic, since the toxin may produce both a direct signal from a specific sensor (detected at step 1820) and an associated biological signal (step 1830), that are blended (or multiplexed) in the invention. Examples of a direct signal from a dedicated sensor (element 1810) are the chemical detection of carbon monoxide, or a biological assay for an infective agent (viruses, bacteria, fungi). Ideally, this sensor operates in near-real time, although this is not a requirement and if not the case will simply provide a slower, non-real time signal. Examples of an associated biological signal to carbon monoxide—a toxin that is traditionally considered “unsensed”—is the specific cherry red colorimetric change of hemoglobin from carbon monoxide and the non-specific reduction in oxygenated hemoglobin that results when carbon monoxide binds to oxygen binding sites.

FIG. 19 further depicts that the enciphered nervous system of the invention forms an associative symbolic representation (step 1820) between the direct and associated biological sensed signals. The symbolic relationship may include a direct mathematical transform, such as a quantitative relationship of the sensed signal to carbon monoxide or the associated biological signal of cherry red discoloration of hemoglobin to biologically relevant concentrations. The symbolic relationship may also use an artificial neural network or other pattern-learning or relational approaches to link, e.g., elevated heart rate or oxygen desaturation to the toxin.

In FIG. 19 step 1840, signals are multiplexed in a non-linear analytical fashion, as defined in the symbolic representation for any specific toxin. Computer logic is then used to control a biological or artificial effector device. Several therapy or monitor functions can be programmed to close a biofeedback loop. For instance, the signal from the normally unsensed toxin can be transduced into a specific signal on a naturally sensed “channel” (step 1860), e.g., low intensity vibration on skin on the nostril (intuitively linked with inhalation), or stimulation of skin over a scalp region normally associated with deoxygenation. This latter biofeedback uses information from training related to the individual person (contributing to the personalized enciphered nervous system), or a database of symbolic representations from many individuals associating related stimuli (here, de-oxygenation) to biological signals. This is an example of a population-based, or potentially crowd-sourced enciphered nervous system. Another biofeedback option is therapeutic (1860)—delivery of an antidote, by sending control signals to a device. For carbon monoxide exposure, therapy includes increasing oxygen concentrations (using hyperbaric oxygen in extreme cases) and administering methylene blue.

Nomograms of the detrimental impact of sensed signals are used to calibrate sensing and delivery of therapy functions from the enciphered nervous system. For carbon monoxide, exposures at 100 ppm (0.01%) or greater can be dangerous to human health. Accordingly, in the United States, Federal agencies such as OSHA put a highest limit on long-term workplace exposure levels of 50 ppm, but individuals should not be exposed to an upper limit (“ceiling”) of 100 ppm. Exposures of 800 ppm (0.08%) lead to dizziness, nausea, and convulsions within 45 min, with the individual becoming insensible within 2 hours. Clearly, detecting this toxin early would have extremely practical implications in industrial environments, for instance. Other nomograms can be developed to identify thresholds for “safe” versus “actionable” exposure to various stimuli including but not limited to chemicals, biological toxins, radiation, electrical stimuli, visual stimuli and auditory stimuli.

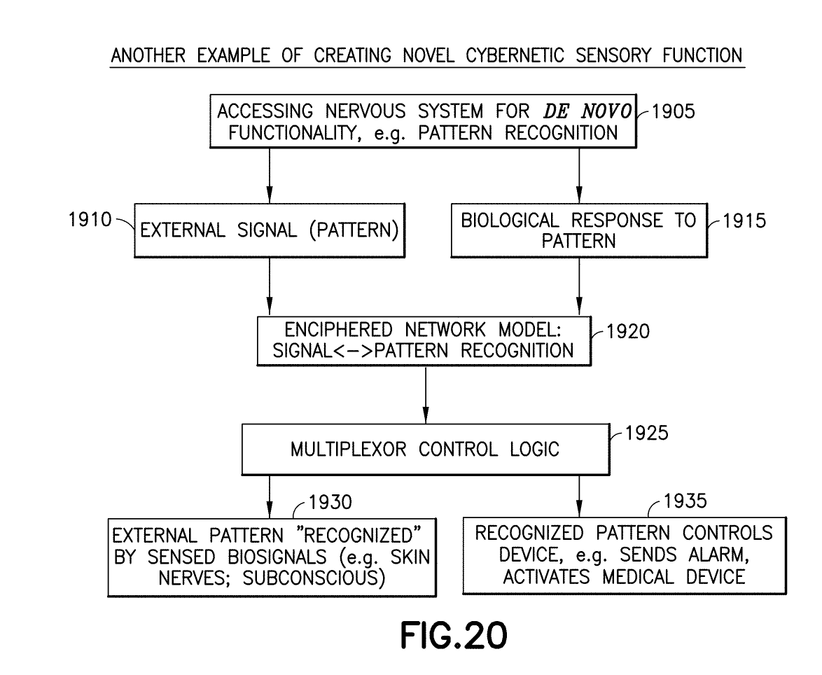

FIG. 20 provides another embodiment using the enciphered network to access to the processing power of the natural nervous system to perform an arbitrary task, such as pattern recognition (step 1905). This embodiment of the invention is based upon 3 concepts. First, that the brain is more efficient at some tasks than even the most powerful and well-programmed artificial electronic computers. Pattern recognition, e.g., facial recognition, is an excellent example that is easily accomplished by most people yet that is suboptimal by computers even with very sophisticated programming. Second, that the brain output from a presented stimulation can be sensed. Third, that the brain has unused capacity that can be accessed for this purpose. For instance, for neural processing, only a minority is used even in highly stressful human activities such as warrior combat (e.g., 40% capacity used). In highly focused, non-life-or-death situations, a minority is still used, likely 20-40%, e.g., NBA finals, SAT testing. Therefore, there is substantial residual capacity at any one time. This third item also presents safety limits, however, and in the case of pattern recognition, the invention must not be used for bioencoding images or data that would be emotionally harmful or sensitive.

Steps 1910 and 1915 link the pattern (e.g., a face) to the biological sensed response—for instance, activity of nerves in the scalp over the parietal lobes of the brain, or over the forehead indicating “recognition”. This is used to create the elements of enciphered nervous system for this task (step 1920). This will be personalized, but can also take inputs from a multi-person (population, crowd-sourced) encyphered nervous system. Once this link has been made, then presentation of the pattern will result in a “sensed” biological pattern, which is used by the multiplexer or control logic in step 1925 to deliver a “1” (recognized) or “0” (not recognized) to control a device (step 1930) (e.g., external computer classifier) or stimulate the individual via a surrogate sensation (step 1935) (e.g., vibration at the left upper arm if a recognized pattern is detected). Uses for this invention include pure biocomputing (pattern recognition of familiar or abstract shapes/codes), formally encoding and enhancing memory of faces for a particular person, and security such that only a hostile pattern/face elicits a specific surrogate sensation or activates a device. One other advantage of this approach over waiting for a cognitive recognition of the pattern is that this can function as a “background process” and/or provide faster pattern recognition.

Thus, this invention can improve and enhance function of traditional senses, if a device is used that integrates sensors that sense outside the normal physiological range can be used to enhance the range of normal physiological sensation. For instance, sensing signals in the “inaudible to humans” part of the frequency spectrum, transducing the signal to the audible range, and transmitting it via bony conduction using a device could be used for private communication, encryption, recreational or other purposes. Medically, this invention could be used to compensate for hearing loss. This same invention with sensors of vibration could be used to compensate for loss of this sensation in certain neurological diseases such as peripheral neuropathy, by transmitting this sensation to an intact sensation in a different part of the body.

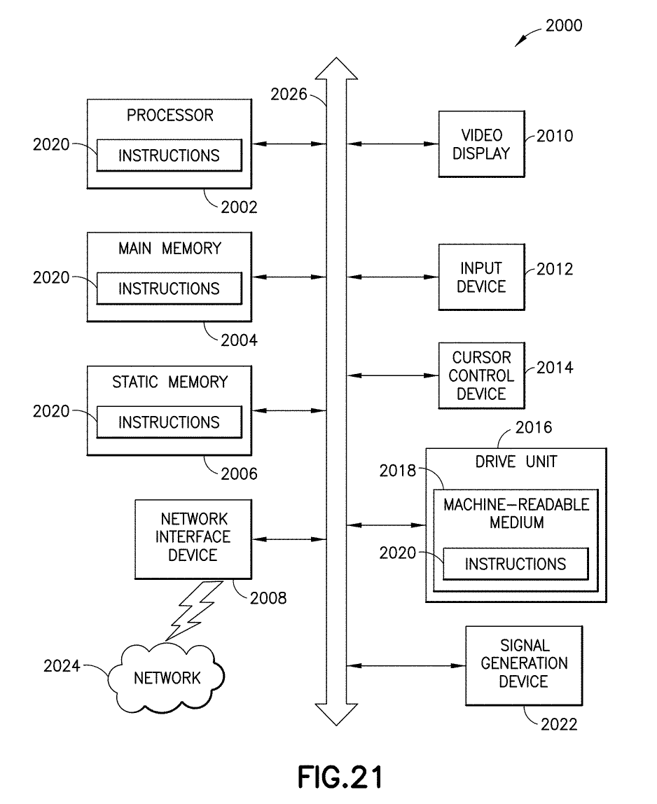

FIG. 21 is a block diagram of an illustrative embodiment of a general computer system 2000. The computer system 2000 can be the signal processing device 114 and the computing device 116 of FIG. 1. The computer system 2000 can include a set of instructions that can be executed to cause the computer system 2000 to perform any one or more of the methods or computer based functions disclosed herein. The computer system 2000, or any portion thereof, may operate as a standalone device or may be connected, e.g., using a network or other connection, to other computer systems or peripheral devices. For example, the computer system 2000 may be operatively connected to signal processing device 114, analysis database 118, and control device 120.

In operation as described in FIGS. 1-21, the modification or enhancement of the nervous system of the body by creating and using an enciphered functional network as described herein can be used to enhance performance in normal individuals or restore or treat lost function in patients.

The computer system 2000 may be implemented as or incorporated into various devices, such as a personal computer (PC), a tablet PC, a personal digital assistant (PDA), a mobile device, a palmtop computer, a laptop computer, a desktop computer, a communications device, a control system, a web appliance, or any other machine capable of executing a set of instructions (sequentially or otherwise) that specify actions to be taken by that machine. Further, while a single computer system 2000 is illustrated, the term “system” shall also be taken to include any collection of systems or sub-systems that individually or jointly execute a set, or multiple sets, of instructions to perform one or more computer functions.

As illustrated in FIG. 21, the computer system 2000 may include a processor 2002, e.g., a central processing unit (CPU), a graphics-processing unit (GPU), or both. Moreover, the computer system 2000 may include a main memory 2004 and a static memory 2006 that can communicate with each other via a bus 2026. As shown, the computer system 2000 may further include a video display unit 2010, such as a liquid crystal display (LCD), a light emitting diode such as an organic light emitting diode (OLED), a flat panel display, a solid state display, or a cathode ray tube (CRT). Additionally, the computer system 2000 may include an input device 2012, such as a keyboard, and a cursor control device 2014, such as a mouse. The computer system 2000 can also include a disk drive unit 2016, a signal generation device 2022, such as a speaker or remote control, and a network interface device 2008.

In a particular embodiment, as depicted in FIG. 21, the disk drive unit 2016 may include a computer-readable medium 2018 in which one or more sets of instructions 2020, e.g., software, can be embedded. Further, the instructions 2020 may embody one or more of the methods or logic as described herein. In a particular embodiment, the instructions 2020 may reside completely, or at least partially, within the main memory 2004, the static memory 2006, and/or within the processor 2002 during execution by the computer system 2000. The main memory 2004 and the processor 2002 also may include computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments, the methods described herein may be implemented by software programs tangibly embodied in a processor-readable medium and may be executed by a processor. Further, in an exemplary, non-limited embodiment, implementations can include distributed processing, component/object distributed processing, and parallel processing. Alternatively, virtual computer system processing can be constructed to implement one or more of the methods or functionality as described herein.

It is also contemplated that a computer-readable medium includes instructions or receives and executes instructions 2020 responsive to a propagated signal, so that a device connected to a network 2024 can communicate voice, video or data over the network 2024. Further, the instructions 2020 may be transmitted or received over the network 2024 via the network interface device 2008.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein,

In a particular non-limiting, example embodiment, the computer-readable medium can include a solid-state memory, such as a memory card or other package, which houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

In accordance with various embodiments, the methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that software that implements the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein, and other equivalents and successor media, in which the software implementations herein may be stored, are included herein.

Thus, a system and method of diagnosis tailoring for an individual, and capable of controlling effectors to deliver therapy or enhance performance, have been described. Although specific example embodiments have been described, it will be evident that various modifications and changes may be made to these embodiments without departing from the broader scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of any of the above-described embodiments, and other embodiments not specifically described herein, may be used and are fully contemplated herein.

The Abstract is provided to comply with 37 C.F.R. § 1.72(b) and will allow the reader to quickly ascertain the nature and gist of the technical disclosure. It is submitted with the understanding that it will not be used to interpret or limit the scope or meaning of the claims.

In the foregoing description of the embodiments, various features are grouped together in a single embodiment for the purpose of streamlining the disclosure. This method of disclosure is not to be interpreted as reflecting that the claimed embodiments have more features than are expressly recited in each claim. Rather, as the following claims reflect, inventive subject matter lies in less than all features of a single disclosed embodiment. Thus the following claims are hereby incorporated into the Description of the Embodiments, with each claim standing on its own as a separate example embodiment.

1. A method for diagnosis tailoring to improve the breathing-health of an individual, comprising:
detecting one or more signals, directly or indirectly, from one or more sensors, the signals associated with breathing at a plurality of points in time, wherein signals that are not breaths are identified as breath-related and non-breath related components of breathing;
tailoring a diagnosis of breathing-health to the individual based upon identifying one or more breaths from the one or more signals, and identifying at least one or more of (i) one or more quantitative indexes of health symptoms and (ii) one or more quantitative indexes of physical examination signs; wherein the diagnosis tailoring is determined using one or more of mathematical rules, mathematical weighting, machine learning, statistical correlation, and applying a threshold of breathing-health; and
providing a representation of the tailored diagnosis at the one or more points in time.

2. The method of claim 1, wherein the threshold of breathing-health is predetermined or dynamic.

3. The method of claim 2, wherein a threshold of breathing health can be tailored dynamically for the individual based upon one or more of recorded patterns in that individual, recorded patterns in other individuals, patient history, population database, population characteristics, machine learning, and disease type.

4. The method of claim 1, wherein the one or more sensors is physically in contact with the body.

5. The method of claim 1, wherein the one or more sensors is not physically in contact with the body.

6. The method of claim 1, wherein the one or more signals are biological signals.

7. The method of claim 1, wherein the one of more signals are non-biological.

8. The method of claim 1, wherein the plurality of points in time comprise one or more days for repeated testing.

9. The method of claim 6, wherein the biological signal is selected from one or more of sounds from the airway associated with breathing, sounds detectable on the body surface associated with breathing, vibrations detectable on the body surface associated with breathing, chest wall movement associated with breathing, abdominal movement associated with breathing, heart rate patterns associated with breathing, alterations in heart output associated with breathing, levels of body oxygenation associated with breathing, body chemistry levels associated with breathing, galvanic skin resistance associated with breathing, brain function associated with breathing and levels of body color associated with breathing.

10. The method of claim 1, wherein the one or more signals is selected from one or more levels of pressure associated with breathing, one or more levels of ambient sound associated with breathing, one or more levels of vibration associated with breathing, one or more levels of temperature associated with breathing, and one or more levels of gas composition associated with breathing, and combinations thereof.

11. The method of claim 1 wherein the quantitative indexes of health symptoms comprise one or more of the STOP-BANG questionnaire and disease survey scores.

12. The method of claim 1 wherein the quantitative indexes of health symptoms comprise one or more of the Epworth Sleepiness Scale score, quality of life survey scores, and symptom survey scores.

13. The method of claim 1 wherein the quantitative indexes of health symptoms comprise one or more measures of the central and peripheral nervous system, cardiovascular system, respiratory system, skeletal muscles and skin.

14. The method of claim 1 wherein the quantitative indexes of physical examination signs comprise components of the STOP-BANG and related scores.

15. The method of claim 1 wherein the quantitative indexes of physical examination signs measure one or more of the central and peripheral nervous system, cardiovascular system, respiratory system, skeletal muscles and skin.

16. (canceled)

17. The method of claim 1, wherein breath-related components comprise one or more of normal breath, cough, snore, and wheeze.

18. The method of claim 1, wherein non-breath-related components comprise one or more of apnea and noise.

19. The method of claim 3, wherein the threshold is dynamic and adapts to or varies with one or more of the signals sensed from the individual over time, the health symptoms change over time, the physical examination signs change over time, and one or more disease states.

20. The method of claim 1, wherein the mathematical weighting is fixed.

21. The method of claim 1, wherein the mathematical weighting is variable.

22. The method of claim 1, wherein the mathematical weighting is selected from spectral methods, stochastic methods, correlation methods, calculus based approaches, geometric based approaches, and combinations thereof.

23. The method of claim 1, wherein mathematical weighting comprises an enciphered functional network represented by symbolic code.

24. The method of claim 23, wherein the symbolic code is a cypher.

25. The method of claim 1, wherein machine learning is affected by iterative analysis when the individual is at times of low breathing-health and when the individual is at times of high breathing-health.

26. The method of claim 1, wherein statistical correlation is performed between signals acquired from the individual and those stored in a database.

27. The method of claim 26, wherein the database can represent signals from this individual over time, signals from different individuals, or a database from multiple individuals.

28. The method of claim 1, wherein the representation is displayed using one or more of a consumer device, a medical device, a computer and a printed representation.

29. A method for diagnosis tailoring to improve the breathing-health of individuals, comprising:
detecting one or more signals from one or more sensors, the signals associated with breathing at a plurality of points in time;
filtering out from said signals, signals or signal components not associated with breathing, using information from one or more sensors which may be the same or different from said sensors that detect said signals;
detecting normal and abnormal breaths from said filtered signals using a combination of mathematical analyses, comparisons against breath events for that individual, comparisons against breath events for other individuals, and known indices of health;
forming a composite representation from one or more of (i) patterns of normal and abnormal breaths, (ii) patterns of known indices of health, at one or more points in time, referenced to known periods of health and disease for a specific individual;
tailoring a diagnosis of breathing-health to the individual based upon said composite representation at one or more points in time; and
providing said tailored diagnosis to guide management of breathing health in said individual.

30. A system for tailoring treatment to improve the breathing-health of an individual, comprising:
a processor;
a memory storing instructions that, when executed by the processor, performs operations comprising:
detecting one or more signals from one or more sensors, the signals associated with breathing at a plurality of points in time;
filtering out from said signals, signals or signal components not associated with breathing, using information from one or more sensors which may be the same or different from said sensors that detect said signals;
detecting normal and abnormal breaths from said filtered signals using a combination of mathematical analyses, comparisons against breath events for that individual, comparisons against breath events for other individuals, and known indices of health;
forming a composite representation from one or more of (i) patterns of normal and abnormal breaths, (ii) patterns of known indices of health, at one or more points in time, referenced to known periods of health and disease for a specific individual;
tailoring a diagnosis of breathing-health to the individual based upon said composite representation at one or more points in time; and
treating the patient based on the tailored diagnosis by delivering one or more effector signals to control one or more body functions associated with breathing-health.
